(19) United States
(12) Patent Application Publication
(10) Pub. No.: US 2026/0043021 A1
(43) Pub. Date: Feb. 12, 2026

(54) HIGH THROUGHPUT CHARACTERIZATION OF BACTERIAL PROMOTERS FROM THEIR HOST ENVIRONMENTS

(71) Applicant: THE REGENTS OF THE UNIVERSITY OF CALIFORNIA, Oakland, CA (US)

(72) Inventors: Tomoya HONDA, Palo Alto, CA (US); Yasuo YOSHIKUNI, Orinda, CA (US)

(21) Appl. No.: 19/065,904

(22) Filed: Feb. 27, 2025

Related U.S. Application Data
(63) Continuation of application No. PCT/US2023/031771, filed on Aug. 31, 2023.
(60) Provisional application No. 63/402,816, filed on Aug. 31, 2022.

(51) Int. Cl. C12N 15/10

(52) U.S. Cl. CPC C12N 15/1065; C12N 15/1086

(57) ABSTRACT

The present invention provides for methods and compositions for recovering high concentrations of transcripts is technically challenging for bacteria colonizing within the host environments. The invention uses Pi-seq technology in a DNA-barcoded promoter library to improve the ability to quantify transcriptional activity of bacterial genes. The invention offers a rapid way to screen and identify biosensors for chemicals and physical conditions associated with host physiology.

### CROSS REFERENCE TO RELATED APPLICATIONS

This application claims priority as a continuation application to PCT International Patent Application No. PCT/US2023/31771, filed Aug. 31, 2023, which claims priority to U.S. Provisional Patent Application Ser. No. 63/402,816, filed Aug. 31, 2022, both of which are incorporated by reference in their entireties.

### STATEMENT OF GOVERNMENTAL SUPPORT

The invention was made with government support under Contract No. DE-AC02-05CH11231 awarded by the U.S. Department of Energy. The government has certain rights in the invention.

### REFERENCE TO SEQUENCE LISTING

The instant application contains a Sequence Listing which has been submitted electronically in XML format and is hereby incorporated by reference in its entirety. Said XML copy, created on 3 Oct. 2025, is named “2022-081-03 Sequence Listing.xml” and is 229,450 bytes in size.

### FIELD OF THE INVENTION

This invention relates generally to characterizing bacteria from their host environments.

### BACKGROUND OF THE INVENTION

Plant roots exudate rich nutrients to the soil environments creating unique habitats for diverse microorganisms. The rhizobacterial community exchanges their metabolites and plays important roles for plant physiology. In recent years, there has been enormous progress in metagenomic studies to uncover the rhizobacterial compositions across diverse plant species, genotypes and soil environments. Although the catalog of taxonomy and genomic information has unprecedentedly increased, much less is studied about their physiology.

One approach to investigate rhizobacterial physiology is to measure their transcriptional activity. Traditional approaches, using fluorescent reporters and qPCR, allow to measure a handful of promoter activities. However, utilizing genome-wide transcriptomics is challenging for the plant-associated bacteria, because bacterial RNA is significantly outnumbered by the release of host plant RNA during sample preparations. This technical problem, which is common for host-associated bacteria, has been addressed by various approaches such as in vitro cell culturing by root exudates, enrichment of bacterial mRNA by using customized probes, and physical separation of bacterial cells from host tissues. However, these methods require long procedures of sample preparations and it still remains as a challenge to make the transcriptomic profiling efficient and scalable.

### SUMMARY OF INVENTION

The present invention provides for a method to screen and identify biosensors for chemicals and physical conditions associated with a host cell's physiology, such as by quantifying transcriptional activity of a library of host cell promoters.

The present invention provides for a method to quantifying transcriptional activity of promoters, the method comprising: (a) extracting a library of 5′-regulatory regions (such as about 140 bp region) from a genome; (b) constructing a barcoded promoter library from the library of 5′-regulatory regions, wherein each member of the barcoded promoter library comprises a construct comprising from 5′ to 3′: 5′-regulatory region-barcode-reporter gene; (c) inserting each member of the barcoded promoter library into a chromosome of a host cell using chassis-independent recombinase-assisted genome engineering (CRAGE) to produce a library of host cell containing the construct; (d) testing the library of host cells to a test condition; (e) identifying the promoters that are up-regulated or down-regulated in response to the test condition; and (f) optionally identifying each gene corresponding to each identified up-regulated or down-regulated promoter in the host cell or in nature. CRAGE is described and taught in PCT International Patent Application No. PCT/US2017/014788 and U.S. Pat. No. 11,674,145, which are both herein incorporated by reference in their entireties.

In some embodiments, the test condition is a change in the environment. In some embodiments, the change in the environment is introduction to the proximity of a host organism or infection of a host organism. In some embodiments, the host organism is a plant. In some embodiments, the introduction to the proximity of the host organism is the introduction of the host cell to a root region of a plant. In some embodiments, the 5′-regulatory region is about 140 bp in length. In some embodiments, the reporter gene is green fluorescent protein (GFP).

In some embodiments, the host cell colonizes a host organism's environment, such as in the environment around a plant's roots. In some embodiments, the method comprises recovering a high concentration of transcripts for host cells colonizing within a host organism's environment. To improve the ability to quantify transcriptional activity of bacterial genes, a technology named Pi-seq is developed that uses a DNA-barcoded promoter library. In some embodiments, the method provides a means to rapidly screen and identify biosensors for chemicals and physical conditions associated with host physiology. In some embodiments, the host cell is or is a potential plant pathogen. In some embodiments, the phenotype is an ability to infect or reside on a plant, such as in or on the root of the plant.

The present invention provides for nucleic acid comprising (i) a promoter up-regulated or down-regulated in response to a host cell colonizing for about one to three days of a plant seedling, such as an Arabidopsis seedling, and (ii) a vector element heterologous to the host cell, or a reporter gene operatively linked to the promoter. Exemplary up-regulated promoters include SEQ ID NOs:83-196. Exemplary down-regulated promoters include SEQ ID Nos:1-82.

The present invention provides for nucleic acid comprising (i) a promoter up-regulated or down-regulated in response to infection of a plant by a host cell, such as infection of Arabidopsis thaliana by Pseudomonas syringae, and (ii) a vector element heterologous to the host cell, or a reporter gene operatively linked to the promoter. Exemplary up-regulated promoters include SEQ ID NOs:211-228. Exemplary down-regulated promoters include SEQ ID Nos:202-210.

The present invention provides for a library of nucleic acids of the present invention.

The present invention provides for methods and compositions described herein.

Recovering high concentrations of transcripts is technically challenging for bacteria colonizing within the host environments. To improve the ability to quantify transcriptional activity of bacterial genes, we developed a technology named Pi-seq that uses a DNA-barcoded promoter library. This method offers a rapid way to screen and identify biosensors for chemicals and physical conditions associated with host physiology.

(1) Approach: Recovering high concentrations of transcripts is technically challenging for bacteria colonizing within the host environments. To improve the ability to quantify transcriptional activity of bacterial genes, we developed a technology named PiSeq that uses a DNA-barcoded promoter library. As a proof of concept, we demonstrated this approach by using Pseudomonas simiae WCS417 which colonizes plant roots. For this strain, we computationally extracted the promoter regions from the genome information, then generated a DNA-barcoded promoter library. Next we integrated the library into the chromosome by CRAGE. When transcription occurs from the artificially introduced promoters, RNA with the barcode is produced. After the nucleic acid extraction, they are amplified by targeted PCR (thus enriching bacterial transcripts hidden by plant RNA), and then quantified by sequencing. PiSeq also offers a rapid way to screen and identify biosensors for chemicals and physical conditions (e.g., pH, temperature).

(2) Pseudomonas simiae WCS417 strain was used as host strain. DNA library is designed to contain promoter regions (defined as 140 bp upstream of each coding DNA sequence [CDS]) with 23 bases of random DNA barcodes. The barcoded promoter library was integrated into the genome using CRAGE technology. Colonization experiment was conducted by using the engineered strains and two-week old Arabidopsis thaliana. They were incubated at 22 C for 3-5 days. When infection assay was conducted, Pseudomonas syringae was applied onto Arabidopsis leaves. For the sampling process, roots were harvested and nucleic acids (DNA, RNA) were extracted. RNA samples were converted to cDNA, and the barcode region is PCR-amplified with targeted primers, then read by a next-generation sequencer. For quantification, a similar process is repeated for the barcodes in genomic DNA. Afterward, RNA over DNA barcode counts for each gene is calculated as a proxy of promoter activity. The promoter activity was compared with native promoter activity by performing RNAseq, comparing the relative expression across different culture conditions.

(3) Results: The activities of about 2900 promoters out of total 5506 promoters were reproducibly characterized. A majority of promoters exhibited native promoter activities based on the analysis against RNAseq. As a preliminary experiment, we traced promoter activity changes during early colonization (for 3 days) on Arabidopsis seedlings. We identified 114 up-regulated and 82 down-regulated promoters. A pathway analysis revealed biofilm genes are upregulated, while metabolism-related genes (ribosomes, amino acids and lipids synthesis) are down-regulated. We also identified 18 promoters that were upregulated when Arabidopsis thaliana got infected by Pseudomonas syringae. They include the promoters encoding to detect metabolites such as xanthine and taurine.

(1) Health diagnosis: Existing methods to analyze biomarkers (eg., cancer detection, preterm birth, newborn health) from human biopsy and blood samples require time and cost. The identified biosensors from metagenomes can be enclosed in an in-vitro transcription system which produces output results efficiently and cheaply.

(2) Environmental monitoring: Soils and drinking water need to be regularly tested for possible contaminations of toxic chemicals like metals. The identified biosensors from metagenomes may also detect these chemicals.

(3) Beauty and Health: Skin bacteria may contain biosensors that recognize skin conditions. We can add synthetic circuits to the downstream of the identified promoters to treat skin before the symptoms become worse.

(4) Agriculture: Soil bacteria may contain biosensors that recognize different plant species and plant physiology. We can add synthetic circuits to the downstream of the identified promoters to increase crop production. The examples include toxin production to certain weeds, ethylene production when plants get infected by pathogens.

(5) Drugs: Gut bacteria play important roles in human physiology. We can search for unique biosensors associated with certain diseases.

The metagenomic resources are very rich yet their functions are unexplored. The Pi-seq platform allows the high throughput characterization of putative promoter activities, overcoming low cell issues in their native environments.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1: Overview of experimental design. (A) 140 bp sequences upstream of protein coding regions were computationally extracted for all annotated genes in P. simiae WCS417 genome. The library of DNA sequences was divided into three groups based on the length of their intergenic regions towards upstream genes (group I: 1-30 bp, group II: 30-140 bp, and group III: >140 bp). (B) For each group, the DNA library was synthesized, assembled with barcodes (23 bp) and cloned into a shuttle vector (modified pW26) upstream of a super-folding GFP (sfGFP). These regions plus an apramycin-resistant marker (Apr^{R}) were flanked by two mutually exclusive lox sites (lox2272 & lox5171) necessary for recombination. (C) The plasmids were then transformed into a donor E. coli strain and conjugated into a domesticated P. simiae WCS417 strain (strain SB599). The recipient strain harbors a landing pad in the chromosome with Cre recombinase gene and kanamycin-resistant marker (Km^{R}) flanked by the same two mutually exclusive lox sites (lox2272 & lox5171). Via Cre recombinase activity (CRAGE), the DNA sequences flanked by the lox sites on the plasmids were integrated into the landing pad loci in P. simiae WCS417. (D) To characterize the library transcriptional activity, cell populations were grown under various conditions such as in liquid media and in association with host plants. After the assay, the cell samples were collected and nucleic acids were extracted. (E) The barcode regions were amplified by targeted PCR from both genomic DNA and cDNA synthesized from RNA. Subsequently, the barcode counts were analyzed from sequencing data and promoter activities were quantified by normalizing the barcode counts from RNA by DNA for each gene.

FIG. 2: Characterization of library coverage and promoter activity in liquid media. (A) Overview of the experiments. The library cell populations were grown in 20 mM glucose and citrate. Sequence libraries for Pi-seq and RNA-seq were created for each of the conditions (n=2) and analyzed to produce the figures as indicated. (B-E) Analysis of Pi-seq data for the cells grown in 20 mM glucose. There was a narrow distribution and high coverage of the designed library based on DNA barcode counts (B), while there is a large variation of the promoter activity based on RNA barcode counts normalized by DNA barcode counts for each promoter (C). The group I library (blue bars), which has short intergenic length to the upstream gene, showed a shifted distribution to the lower values, while the other two libraries (green and magenta bars) contain a larger number of promoters with high activity. The coverage of promoter libraries was highly reproducible across two replicates (D) and the promoter activity was also reproducible except the group I library which had lower activity (E). Pearson's r correlation values are shown in the parentheses. (F-I) The same set of analysis on Pi-seq data for the cells grown in 20 mM citrate. The data show a high similarity to the data obtained in glucose. (J,K) Comparison of Pi-seq data against RNA-seq across genes for the data in glucose (J) and citrate (K). Promoter activity obtained from Pi-seq showed a correlation with RNA expression to some extent for the group II and III libraries (green and magenta points), while there was little correlation for the group I library (blue points). (L) Comparison of the two methods for the fold changes of promoter activity and RNA expression. The fold changes were calculated by analyzing the expression in citrate condition divided by that in glucose condition for each method. The promoters that exhibited large fold changes (|log₂FC|>3) showed a quantitative agreement with the data from RNA-seq (highlighted by black circles and a linear fitting by a dashed line). (M) Illustrations of top up-/down-regulated promoters together with the downstream genes. The extracted 140 bp promoter regions are highlighted by a pink background. Consistent with the experimental conditions, the most up-regulated promoter drives the genes for tripartite tricarboxylate transporters, while the top two down-regulated promoters drive glucose transporting genes in the same operon.

FIG. 3: Pi-seq measurements of promoter activity during Arabidopsis root colonization. (A) P. simiae WCS417 cell populations with promoter library group II and III were spread to vertically oriented phytagel plates with 10 day old Arabidopsis thaliana seedlings. The group I library was omitted from the experiment due to their low activity as shown in FIG. 2. After allowing initial colonization for three days, the root samples were collected every 24 hours over 2 days and the sequencing libraries were prepared by targeted PCR of barcodes from the extracted DNA and RNA to quantify the library coverage and promoter activity (n=4 at each sampling). (B-E) Analysis of Pi-seq data for the cells collected from Arabidopsis roots on sampling day 1. Similar to the liquid media experiments in FIG. 2, there was a narrow distribution and high coverage of the library (B) and a large variation of promoter activity (C). The reproducibility across replicates was sufficiently high for both the coverage of promoters (D) and promoter activity (E). (F) Log₂ fold changes in the activity of individual promoters with respect to the sampling day 1. The lines highlighted by red and blue colors indicate the up/down-regulated promoters that were taken as statistically significant (|log₂FC|>1 and Benjamini-Hochberg adjusted p-value<0.1). (G) Histograms for the predicted number of genes that are driven by the identified promoters. In both up-regulated (top) and down-regulated (bottom) promoters, a majority of promoters drive single genes, but some are predicted to drive multiple downstream genes. (H) KEGG pathway analysis based on the list of predicted genes driven by the identified promoters. The pathway encoding biofilm formation was significantly enriched from the list of up-regulated genes (top), while the pathways involved in metabolic functions were enriched from the down-regulated genes (bottom). The dash line indicates a threshold for the statistical significance (adjusted p-value<10⁻⁵).

FIG. 4: Comparison of promoter activity change to colonization efficiency.

FIG. 5: Characterization of library coverage and promoter activity in other culture conditions. In addition to the data shown in FIG. 2, we further analyzed Pi-seq data for the cells growing in three other culture conditions (n=2). From left to right, the results are shown for the cells grown in 40 mM glycerol at higher temperature 37° C., synthetic root exudate medium (see Method section for detail), and 40 mM glycerol. The obtained data establish the high coverage of promoter library and reproducibility of promoter activity measurements. (A-C) Promoter coverage based on DNA barcode counts. (D-F) Promoter activity based RNA barcode counts normalized by DNA barcode counts for each promoter. (G-I) Reproducibility of promoter coverage across two biological replicates. (J-L) Reproducibility of promoter activity across two biological replicates.

FIG. 6: Comparison of Pi-seq to RNA-seq in other culture conditions. In addition to the data shown in FIG. 2, we further compared Pi-seq data (n=2) to RNA-seq data (n=2) for the cells growing in three other culture conditions. From left to right, the results are shown for synthetic root exudate medium (see Method section for detail), 40 mM glycerol, and 40 mM glycerol at higher temperature 37° C. (A-C) Comparison of promoter activity obtained from Pi-seq to RNA expression obtained from RNA-seq across genes. Same as the data shown in FIG. 2, group II and III libraries (green and magenta points) showed some correlation, but little correlation was seen for the group I library (blue points). (D-F) Comparison of the two methods for the fold changes of promoter activity and RNA expression. The fold changes were calculated by dividing the expression values for each of the conditions by the values obtained in 20 mM glucose minimal medium. The x-axis shows the relative promoter activity based on Pi-seq data and the y-axis shows the relative RNA expression based on RNA-seq. For each condition, we saw high agreement of two methods particularly for the promoters that exhibited large fold changes (|log₂FC|>3). They are highlighted by black circles and fitted by a dashed line. (G-I) Illustrations of top up-/down-regulated promoters in each condition as compared to the growth in glucose minimal medium.

FIG. 7: Comparison of Pi-seq to RNA-seq in root colonization assay. (A) Comparison of the promoter activity obtained from Pi-seq (n=4) to the RNA expression obtained from RNA-seq (n=3) across genes in the colonization day 1 samples. There was some extent of correlation for the group II and III libraries (green and magenta points). The group I library was omitted for this experiment because of the low transcriptional activity. (B) Fold changes of promoter activity against RNA expression. The fold changes were calculated by dividing the expression values in the colonization day 1 samples by the values obtained in glucose minimal medium. The x-axis shows the relative promoter activity based on Pi-seq data and the y-axis shows the relative RNA expression based on RNA-seq. Same as liquid culture experiments, there was a high agreement between the two methods particularly for the promoters that exhibited large fold changes (|log₂FC|>3). They are highlighted by black circles and fitted by a dashed line. (C) Top three up-/down-regulated promoters in the colonization day 1 samples as compared to the growth in glucose minimal medium.

FIG. 8: A procedure for the PiSeq.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used in the specification and the appended claims, the singular forms “a,” “an,” and “the” include plural referents unless the context clearly dictates otherwise. Thus, for example, reference to “molecules” includes a plurality of a molecule species as well as a plurality of molecules of different species.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to a value including 10% more than the stated value and 10% less than the stated value.

As used herein, the term “promoter” refers to a polynucleotide sequence capable of driving transcription of a DNA sequence in a cell. Thus, promoters used in the polynucleotide constructs of the invention include cis- and trans-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. Promoters are located 5′ to the transcribed gene, and as used herein, include the sequence 5′ from the translation start codon.

A polynucleotide or amino acid sequence is “heterologous” to an organism or a second polynucleotide or amino acid sequence if it originates from a foreign species, or, if from the same species, is modified from its original form. For example, when a polynucleotide encoding a polypeptide sequence is said to be operably linked to a heterologous promoter, it means that the polynucleotide coding sequence encoding the polypeptide is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety, or a gene that is not naturally expressed in the target tissue).

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a DNA or RNA sequence if it stimulates or modulates the transcription of the DNA or RNA sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “host cell” of “host organism” is used herein to refer to a living biological cell that can be transformed via insertion of an expression vector.

The terms “expression vector” or “vector” refer to a compound and/or composition that transduces, transforms, or infects a host cell, thereby causing the cell to express nucleic acids and/or proteins other than those native to the cell, or in a manner not native to the cell. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in the present invention include those into which a nucleic acid sequence can be inserted, along with any preferred or required operational elements. Further, the expression vector must be one that can be transferred into a host cell and replicated therein. Particular expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The terms “polynucleotide” and “nucleic acid” are used interchangeably and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); positive backbones; non-ionic backbones, and non-ribose backbones. Thus, nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

(1) Approach: Recovering high concentrations of transcripts is technically challenging for bacteria colonizing within the host environments. To improve the ability to quantify transcriptional activity of bacterial genes, we developed a technology named PiSeq that uses a DNA-barcoded promoter library. As a proof of concept, we demonstrated this approach by using Pseudomonas simiae WCS417 which colonizes plant roots. For this strain, we computationally extracted the promoter regions from the genome information, then generated a DNA-barcoded promoter library. Next we integrated the library into the chromosome by CRAGE. When transcription occurs from the artificially introduced promoters, RNA with the barcode is produced. After the nucleic acid extraction, they are amplified by targeted PCR (thus enriching bacterial transcripts hidden by plant RNA), and then quantified by sequencing.

(2) Brief methods and materials: Pseudomonas simiae WCS417 strain was used as host strain. DNA library is designed to contain promoter regions (defined as 140 bp upstream of each coding DNA sequence [CDS]) with 23 bases of random DNA barcodes. The barcoded promoter library was integrated into the genome using CRAGE technology. Colonization experiment was conducted by using the engineered strains and two-week old Arabidopsis thaliana. They were incubated at 22 C for 3-5 days. When infection assay was conducted, Pseudomonas syringae was applied onto Arabidopsis leaves. For the sampling process, roots were harvested and nucleic acids (DNA, RNA) were extracted. RNA samples were converted to cDNA, and the barcode region is PCR-amplified with targeted primers, then read by a next-generation sequencer. For quantification, a similar process is repeated for the barcodes in genomic DNA. Afterward, RNA over DNA barcode counts for each gene is calculated as a proxy of promoter activity. The promoter activity was compared with native promoter activity by performing RNAseq, comparing the relative expression across different culture conditions.

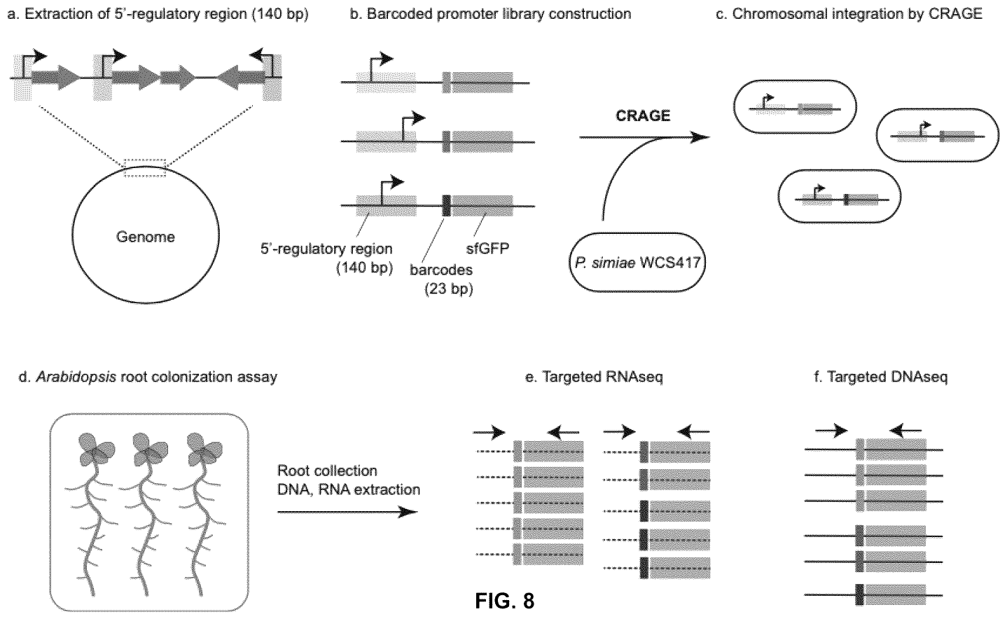

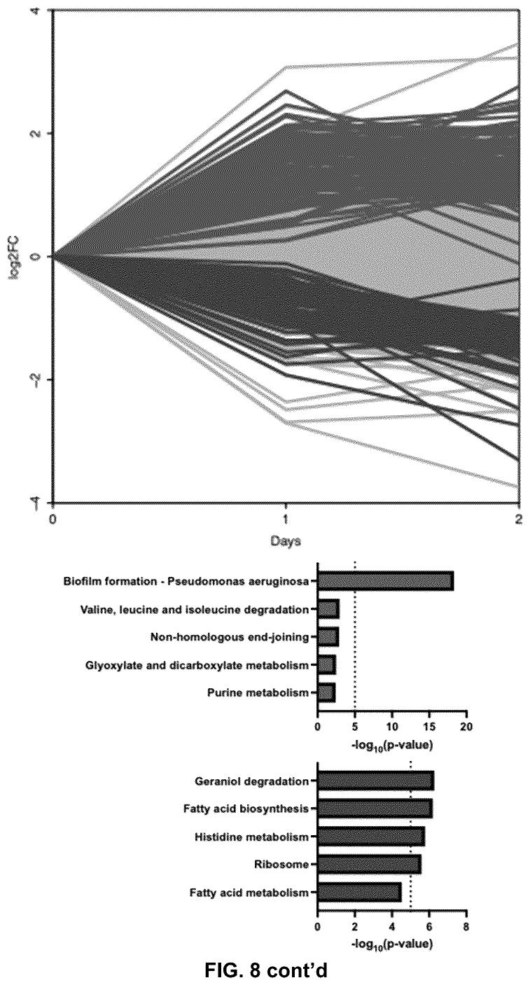

(3) Results: The activities of about 2900 promoters out of total 5506 promoters were reproducibly characterized. A majority of promoters exhibited native promoter activities based on the analysis against RNAseq. As a preliminary experiment, we traced promoter activity changes during early colonization (for 3 days) on Arabidopsis seedlings. We identified 114 up-regulated and 82 down-regulated promoters. A pathway analysis revealed biofilm genes are upregulated, while metabolism-related genes (ribosomes, amino acids and lipids synthesis) are down-regulated. We also identified 18 promoters that were upregulated when Arabidopsis thaliana got infected by Pseudomonas syringae. They include the promoters encoding to detect secondary metabolites such as xanthine and taurine. FIG. 8 shows a procedure for the PiSeq.

Proof of concept research and functionality to characterize promoter activities for the engineered strain collected from Arabidopsis roots has been established. Current work includes identifying transcriptional start sites (TSSs) and extracting longer regulatory regions for creating promoter libraries to increase the coverage and accuracy of promoter activity. This will ultimately result in tool development to identify biosensors (promoters, transcriptional factors) that can detect various physiological and chemical signals from hosts and environments that bacteria are associated with. Potential applications have been thought of and targeted proof of concept research for these applications will be implemented shortly.
(1) Health diagnosis: Existing methods to analyze biomarkers (eg., cancer detection, preterm birth, newborn health) from human biopsy and blood samples require time and cost. The identified biosensors from metagenomes can be enclosed in an in-vitro transcription system which produces output results efficiently and cheaply.
(2) Environmental monitoring: Soils and drinking water need to be regularly tested for possible contaminations of toxic chemicals like metals. The identified biosensors from metagenomes may also detect these chemicals.
(3) Beauty and Health: Skin bacteria may contain biosensors that recognize skin conditions. We can add synthetic circuits to the downstream of the identified promoters to treat skin before the symptoms become worse. (living therapeutics)
(4) Agriculture: Soil bacteria may contain biosensors that recognize different plant species and plant physiology. We can add synthetic circuits to the downstream of the identified promoters to increase crop production. The examples include toxin production to certain weeds, ethylene production when plants get infected by pathogens.
(5) Drugs: Gut bacteria play important roles in human physiology. We can search for unique biosensors associated with certain diseases.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Transcriptional Profiling of Plant-Root Colonizing Bacteria by CRAGE-MPRA

To this objective, synthetic biology offers an alternative approach. A massively parallel reporter assay (MPRA), sequencing-based quantification of promoter activities using DNA barcodes as reporters, allows the characterization of thousands of promoter activities in a simple workflow. This approach has been applied to model prokaryotic and eukaryotic organisms that have high efficiency of DNA library integration and uncovered the unique features of promoter regulatory elements. In the present study, we employed MPRA in a non-model soil bacterium Pseudomonas simiae WCS417. We utilized CRAGE technology to generate a population with a barcoded promoter library and developed an assay to characterize their in planta promoter activity, which we termed as CRAGE-MPRA. We identified a set of unique regulated promoters during plant-root colonization and verified the deletions of these genes affect the colonization efficiency. The framework developed here allows rapid characterizations of transcriptional activities and is applicable to diverse bacterial species, thereby increasing an opportunity to understand their gene regulation and physiology in native environments.

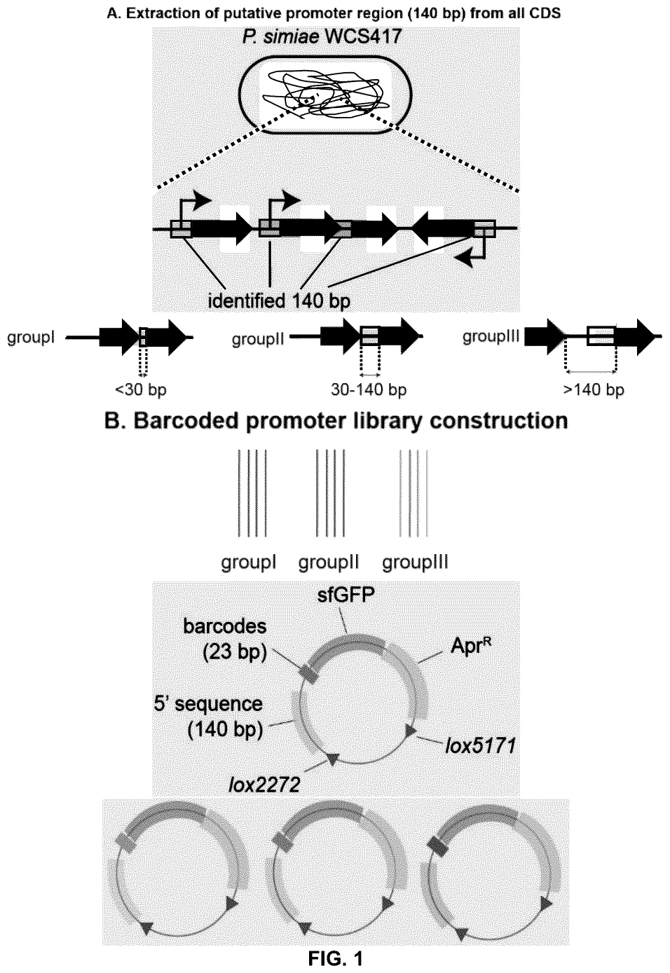

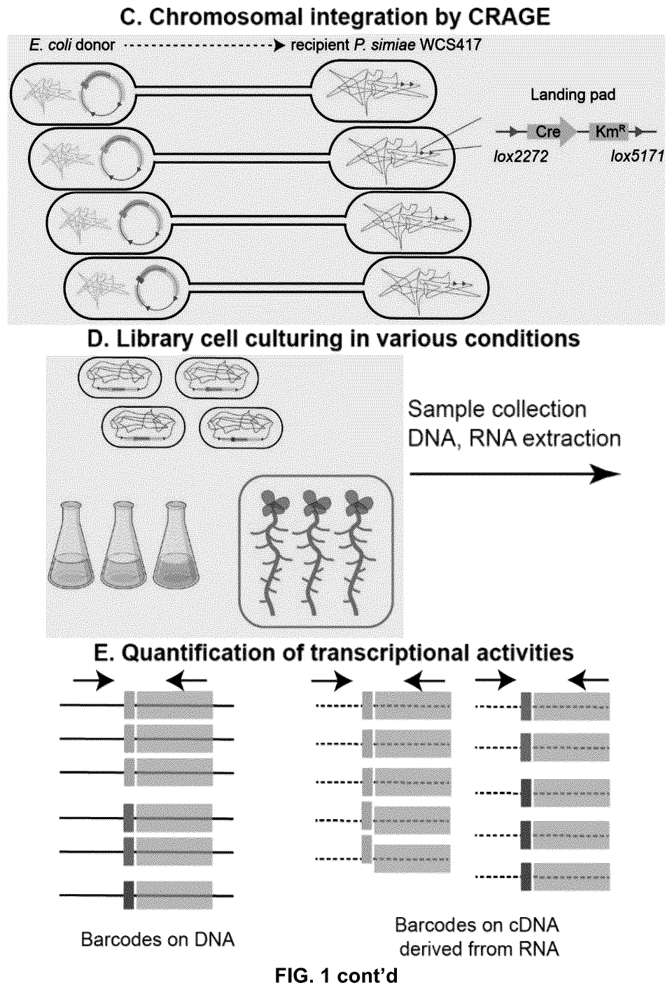

Explanation of Piseq method. To generate a barcoded promoter library, we first computationally extracted 140 bp upstream regions from all annotated protein coding sequences in P. simiae WCS417 genome (FIG. 1). For flexible use, we divided the DNA library into three groups based on the length of intergenic regions to the upstream genes (group I: 1-30 bp, group II: 30-140 bp, and group III: >140 bp). The DNA library was separately synthesized for each group, assembled with barcodes, and cloned into a shuttle vector (modified pW26) upstream of a super-folding GFP (sfGFP) (FIG. 1). We then transformed the vectors into a conjugational E. coli strain and integrated into a landing pad region in the chromosome of P. simiae via CRAGE (FIG. 1). For the assays, we grew the pooled cell library under various conditions (FIG. 1) and used targeted RNA-seq and DNA-seq to amplify the barcoded region. We then analyzed promoter activities by normalizing the barcode counts derived from RNA by the counts from DNA in each promoter (FIG. 1).

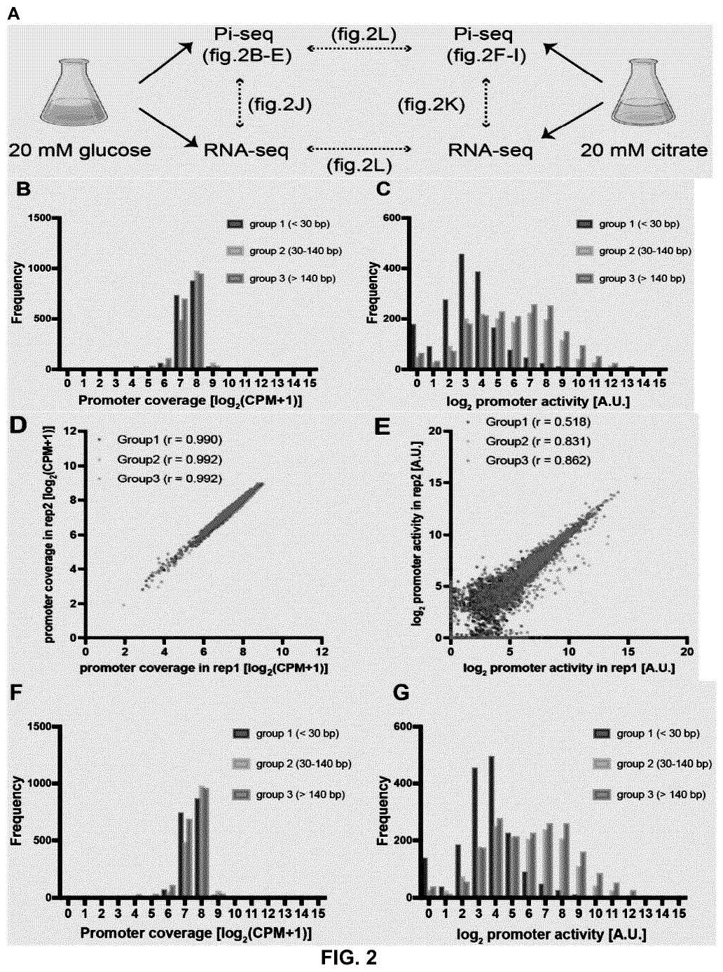

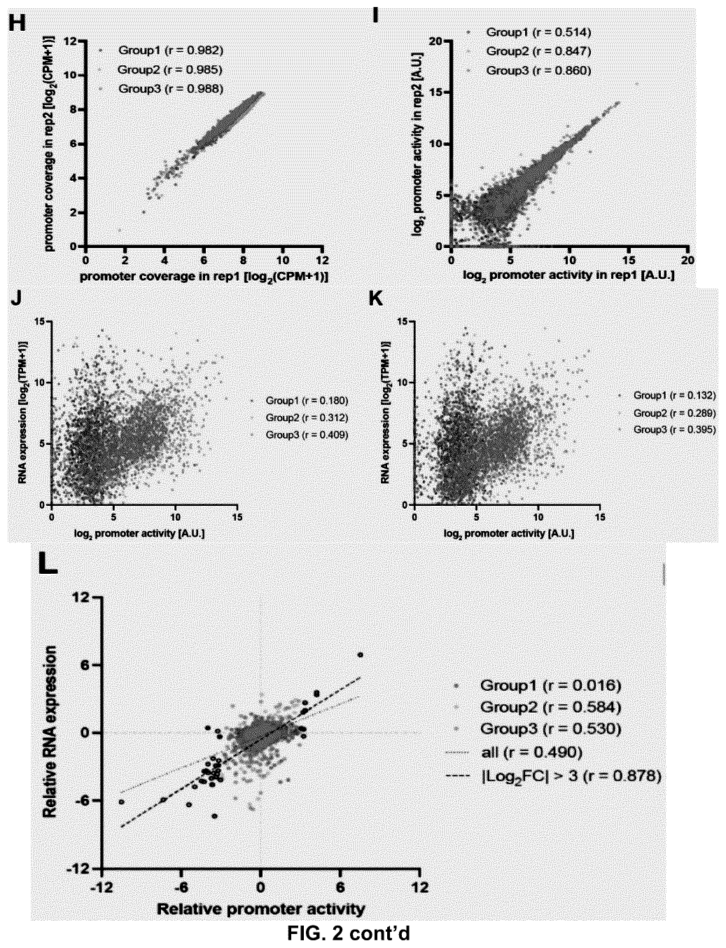

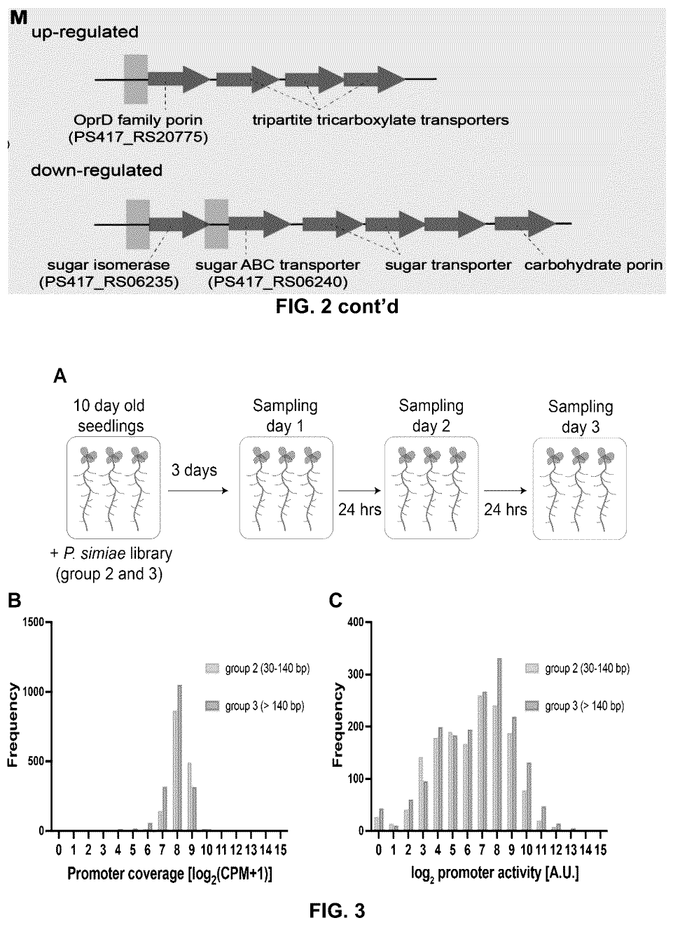

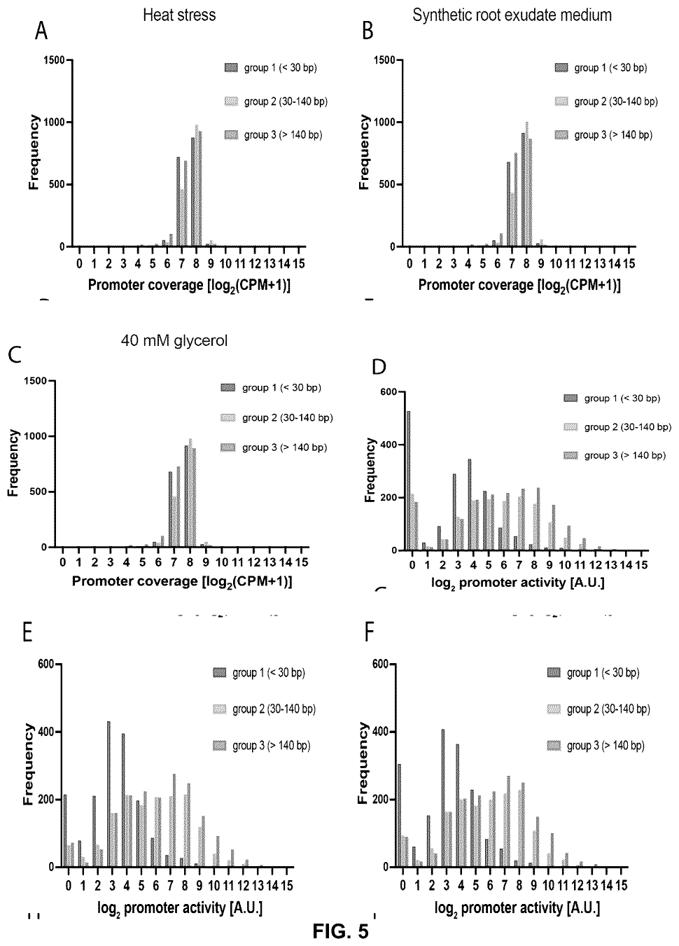

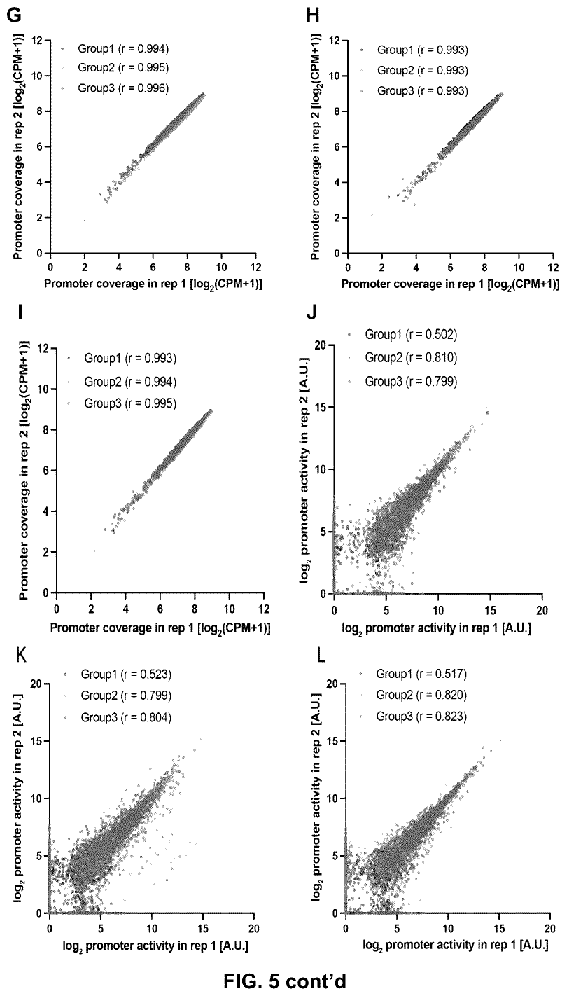

Basic data of promoter activity and reproducibility. To evaluate this approach, we first characterized promoter activities for the pooled group I-III libraries by growing them in defined minimum media supplied with 20 mM glucose or citrate (FIG. 2). In both growth conditions, we observed a high coverage of the designed libraries from the sequencing reads of DNA barcodes (FIG. 2): 97.8% of the total 5090 extracted promoters were covered at least 50 times per million reads. The data were also highly reproducible between two biological replicates (Pearson's r>0.98) (FIG. 2), indicating sufficiently high coverage of the libraries in these culture conditions. On the other hand, the promoter activities, derived by normalizing the count of RNA barcodes by DNA barcodes, showed a high degree of variation with several magnitudes of differences (FIG. 2). Specifically, while the majority of group II & III libraries exhibited high activities (green & magenta bars), the group I library, which has short intergenic regions (below 30 bp), exhibited lower activity, as seen by the shifted distributions (blue bars). Accordingly, the promoter activity in group II & III libraries had a high degree of concordance between the biological replicates (Pearson's r>0.83), but the group I library showed lower reproducibility (Pearson's r˜0.51) (FIG. 2). We reasoned that the majority of promoters in group I library are included as a part of operon and they do not have the active transcription start sites, hence resulting in the observed variability. We obtained similar results by analyzing data for the cells grown in other nutrients or under heat stress conditions (FIG. 5).

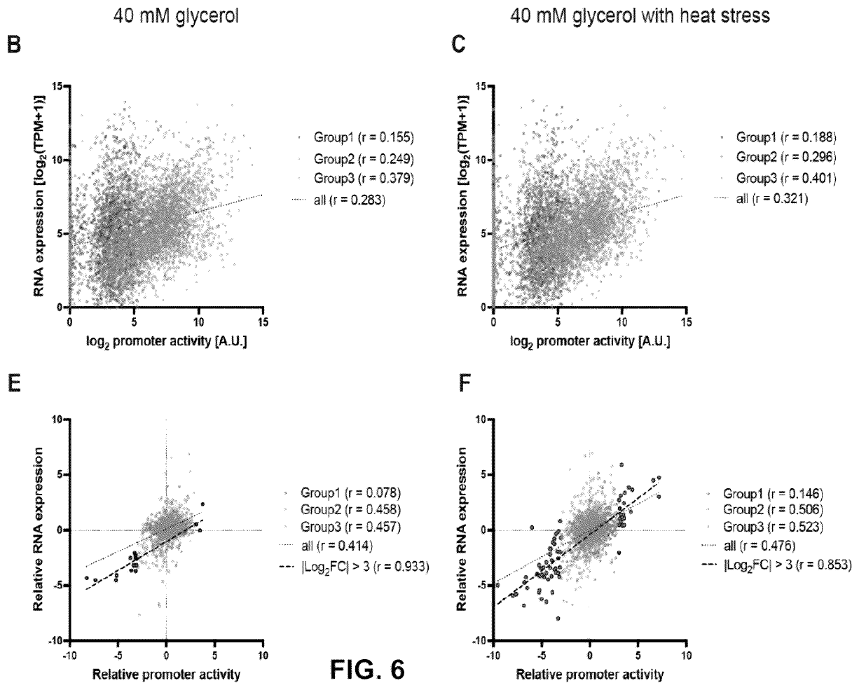

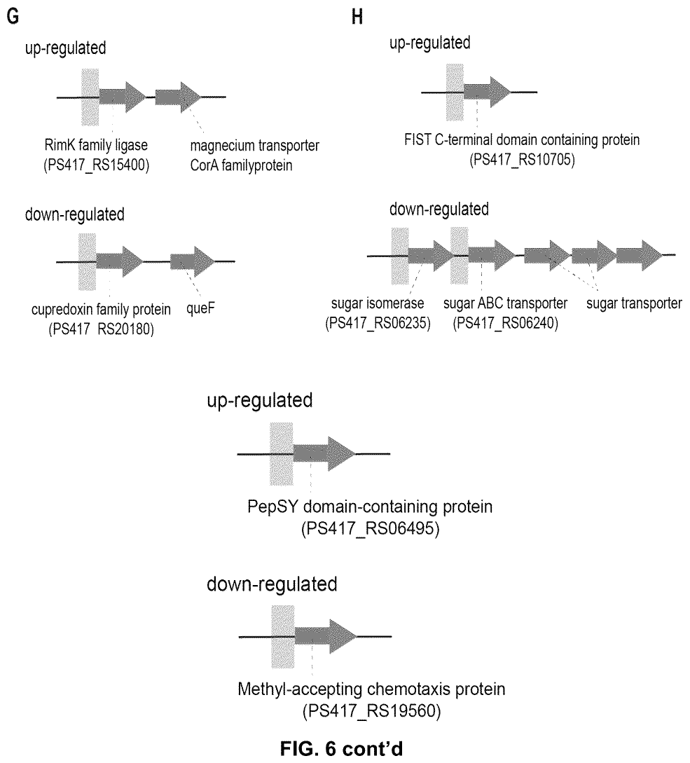

Comparison to RNAseq. To further assess the performance, we next compared this approach with a conventional RNA-seq, using the same RNA samples obtained from minimum media culture experiments. Comparison of the expression across genes showed some degree of correlations between the two methods for the genes in group II & III libraries (FIG. 2). These variations may derive from a limited extraction of promoter region and a chromosomal position effect of promoter activity. On the other hand, the group I library had little correlation with RNA-seq data (FIG. 2), as expected from the lower transcriptional activity. To consider the performance in differential expression, we next compared the fold changes in promoter activity and RNA expression between the two culture conditions (FIG. 2). The relative expression values in each RNA-seq and Pi-seq were calculated by dividing the expression values obtained from citrate culture condition by that from glucose condition. The analysis improved the correlation between the two methods especially in group II & III libraries (green & magenta points). Notably, the promoters that exhibited large fold changes (|log₂FC|>3) quantitatively agreed well with RNA-seq data (highlighted by black circles and a dashed line, Pearson r=0.878). Furthermore, the analysis was able to capture physiologically relevant changes in gene expression (FIG. 2): the two promoters driving glucose binding/transporting genes (RS06235, RS06240) were most down-regulated, while the promoter driving porin and tripartite tricarboxylate transporter (RS20775) was most up-regulated, consistent with the difference in the culture conditions. The quantitative agreement between the two approaches were also verified by conducting the same analysis for other culture conditions (FIG. 6). Overall, these analyses highlight that our method is able to identify a set of promoters that exhibit fold changes according to different culture conditions.

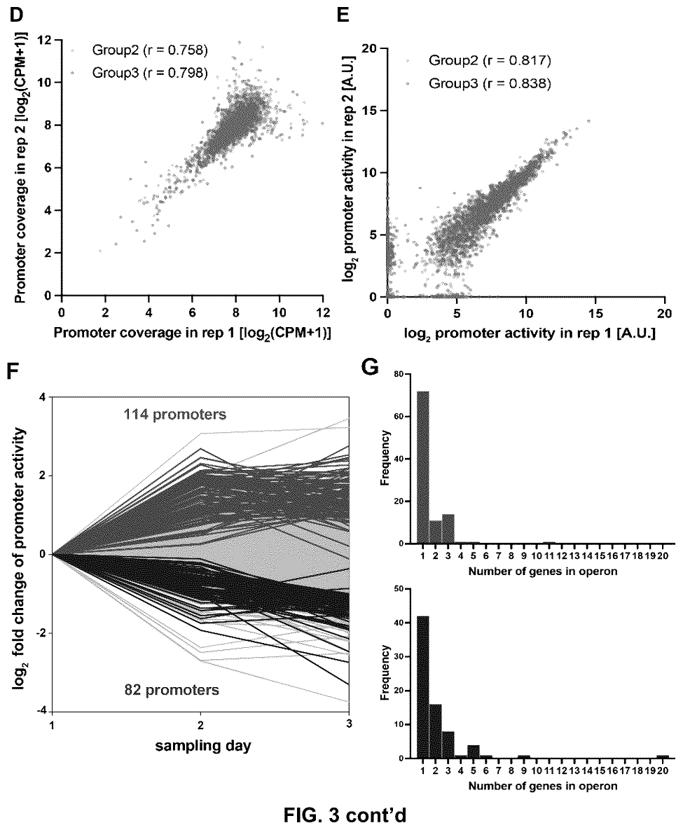

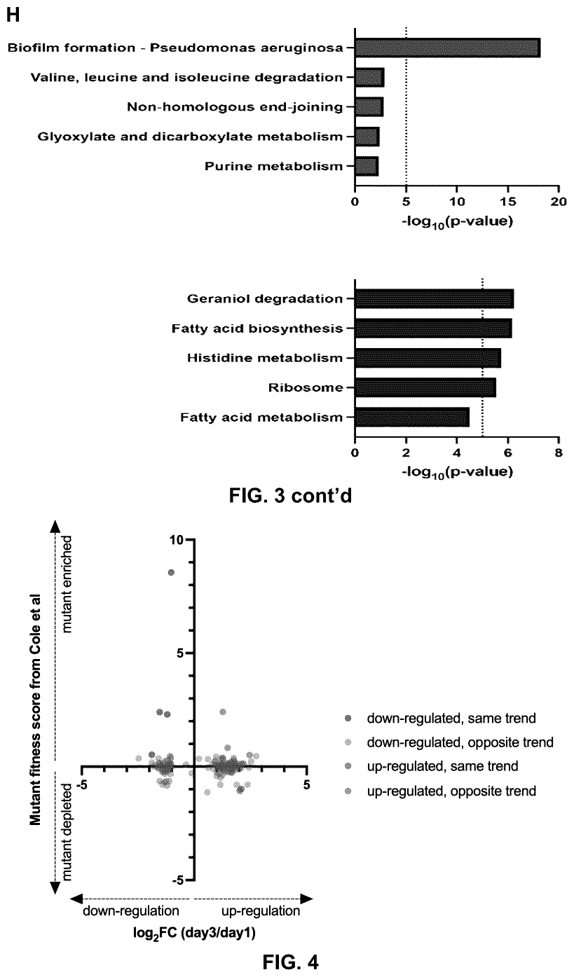

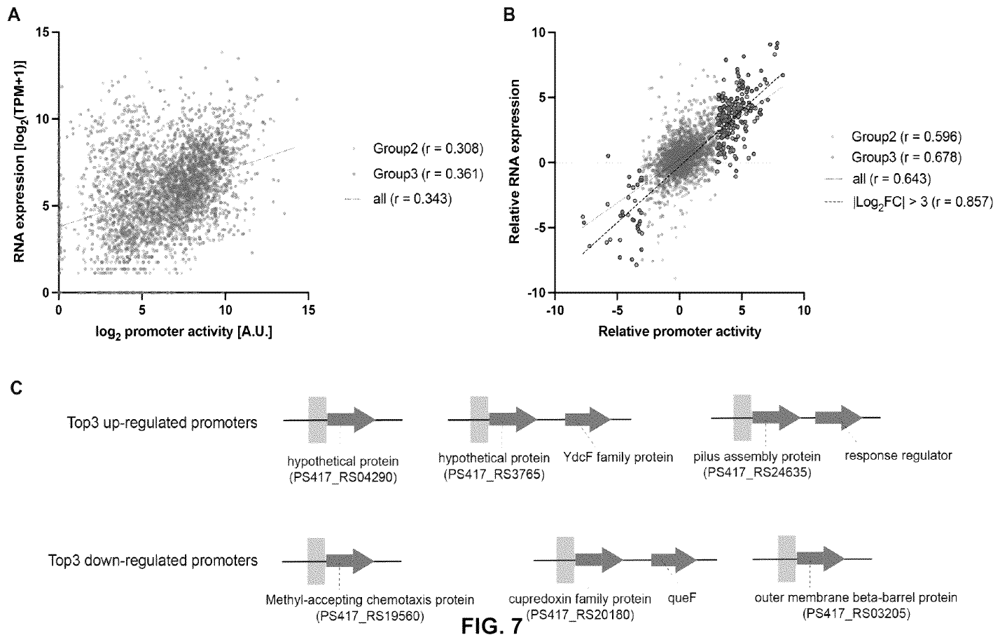

Application to plant assay. Using the cell library, we next probed promoter activity of the P. simiae population during their colonization on Arabidopsis roots. Here we inoculated the pooled cell library of group II & III with 10 days old Arabidopsis seedlings (FIG. 3). After allowing 3 days of initial colonization, we collected the root samples every 24 hours over a 2 days period. We then quantified promoter activity based on the barcode counts from the extracted DNA and RNA. The group I library was omitted because of their low transcriptional activity. Remarkably, while the number of collected cells is ˜100 times lower than liquid culture experiments (see colony formation unit measurement in Method section) and there are contaminated plant root materials, we obtained a sufficiently high coverage of the designed library and reproducibility across biological replicates (FIG. 3), which is similar to liquid culture experiments. This result highlights the utility of this approach, as obtaining sufficient bacterial reads requires extremely high sequencing depth and cost in a conventional RNA-seq due to a plant RNA contamination (Table1). Furthermore, we verified that Pi-seq captures a number of transcriptional changes induced during colonization as compared to liquid culture in glucose (FIG. 7).

Time course analysis. To better understand the colonization process, we next analyzed promoter activity across the time course. We calculated log 2 fold changes of individual promoter activity with respect to the sampling day 1 and identified 114 and 82 promoters that were statistically up-/down-regulated (FIG. 3). To gain the functional views, we examined if the identified promoters drive multiple downstream genes as a structure of operon. For this purpose, we used a software Rockhopper that predicts operon structure based on the P. simiae WCS417 genome and RNA-seq data as inputs. The result shows that while a majority of identified promoters drive single genes, some fraction of promoters drive multiple genes with diverse biological functions (FIG. 3). The identified promoters driving three or more genes are listed (Table2) and the full information is provided in Tables 4-5. To consider the physiologically relevant changes, we carried out KEGG enrichment analysis by feeding the gene lists including the predicted co-transcribed genes as an input. The result showed a pathway encoding for biofilm formation is highly enriched for the genes driven by the up-regulated promoters, while the pathways encoding for metabolic functions such as ribosome, fatty acid and histidine biosynthesis are enriched from the down-regulated promoters (FIG. 3). These results suggest a physiological transition from metabolically active to adhesive and protective states during colonization.

Comparison to Tn-seq. To relate the observed transcriptional changes to phenotypes, we next compared our data with a list of essential genes for root colonization, which are previously identified using randomly barcoded transposon mutagenesis sequencing (RB-TnSeq) in the same strain background. Although we didn't see an uniform pattern by plotting the fold changes of promoter activity against fitness scores of mutants in colonization, we identified a total of 13 genes that exhibited a statistical difference in both promoter activity changes and mutant fitness. We then categorized them into four groups based on the promoter activity change (up or down) and mutant fitness score (mutants depleted or enriched). In particular, Group1 and Group3 showed a consistent pattern for promoter activity and fitness score in colonization: the mutations of the genes driven by down-regulated promoters positively affect colonization (Group1), while the mutations of the genes driven by up-regulated promoters negatively affect colonization efficiency (Group3). The Group1 included the down-regulated promoters driving histidine biosynthesis operon (hisB) and a hypothetical gene, both of which mutations substantially improves root colonization efficiency. Interestingly, however, the genes in Group2 and Group4 showed a contrast pattern which doesn't directly translate the promoter activity into the expected phenotypes. For instance, the Group4 included an up-regulated promoter involved in extracellular polysaccharide and biofilm formation, which were represented from KEGG pathway analysis, but the gene mutation rather increased colonization efficiency.

### DISCUSSION

In this study, we developed MPRA in a rhizobacterium P. simiae WCS417 to characterize their transcriptional activities while colonizing Arabidopsis roots. This approach enables the screening of regulated promoters and can be complementary to a conventional RNA-seq by providing efficiency and scalability without taking care of contaminated plant RNA. Using this approach, we were able to identify a pattern of gene expression and a set of genes that are necessary for root colonization. The strain library developed here can be useful for further examining the mechanisms underlying P. simiae and plant interactions across diverse conditions with biotic and abiotic stresses. Given that CRAGE-MPRA reliably amplifies barcoded transcripts, we can expect it to be applied to lower input samples. Notably, this approach is not limited to the single species but can be easily applied for other bacteria, as long as they are compatible with CRAGE. Therefore, future studies bear the opportunities to explore meta-transcriptome of synthetic bacterial communities in association with their host organisms. The barcoding strategy is advantageous in this context, as it allows discrimination of homologous genes in different organisms, which is a common challenge in metatranscriptome data analysis.

The utility of CRAGE-MPRA can be further improved by promoter library design. Although we took the simplest route to design the library by unbiasedly selecting 140 bp upstream regions of individual genes, it is generally challenging to identify or predict exact bacterial transcriptional start sites (TSS). One approach is to apply a differential RNA-seq that provides a global TSS map through the enrichment of 5′-end transcripts. Performing the differential RNA-seq under various conditions allows to select active promoters that drive operon, which subsequently increases the coverage and quality of sequencing data. Additional consideration is to increase the length of the promoter extraction. In the present study, we selected 140 bp with 30 bp addition to both ends for cloning purposes (total 200 bp), as this length was set by DNA synthesis capability in the manufacturing company. Since the technology keeps updating, we expect increasing the promoter length improves the quality of a library by capturing more regulatory sites.

Although CRAGE-MPRA can be further improved, this method paves the way for screening novel regulatory elements in non-model bacteria. Particularly, little is understood about the physiology of these bacteria in association with host organisms. Since diverse bacteria are found to play roles to maintain host physiology such as protection against infectious diseases, there is a large opportunity to mine novel regulatory elements and ultimately identify biosensors that detect specific physiological signals in plants and animals.

FIG. 5 shows characterization of library coverage and promoter activity in other culture conditions. In addition to the data shown in FIG. 2, we further analyzed Pi-seq data for the cells growing in three other culture conditions (n=2). From left to right, the results are shown for the cells grown in 40 mM glycerol at higher temperature 37° C., synthetic root exudate medium (see Method section for detail), and 40 mM glycerol. The obtained data establish the high coverage of promoter library and reproducibility of promoter activity measurements. (A-C) Promoter coverage based on DNA barcode counts. (D-F) Promoter activity based RNA barcode counts normalized by DNA barcode counts for each promoter. (G-I) Reproducibility of promoter coverage across two biological replicates. (J-L) Reproducibility of promoter activity across two biological replicates.

FIG. 6 shows a comparison of Pi-seq to RNA-seq in other culture conditions. In addition to the data shown in FIG. 2, we further compared Pi-seq data (n=2) to RNA-seq data (n=2) for the cells growing in three other culture conditions. From left to right, the results are shown for synthetic root exudate medium (see Method section for detail), 40 mM glycerol, and 40 mM glycerol at higher temperature 37° C. (A-C) Comparison of promoter activity obtained from Pi-seq to RNA expression obtained from RNA-seq across genes. Same as the data shown in FIG. 2, group II and III libraries (green and magenta points) showed some correlation, but little correlation was seen for the group I library (blue points). (D-F) Comparison of the two methods for the fold changes of promoter activity and RNA expression. The fold changes were calculated by dividing the expression values for each of the conditions by the values obtained in 20 mM glucose minimal medium. The x-axis shows the relative promoter activity based on Pi-seq data and the y-axis shows the relative RNA expression based on RNA-seq. For each condition, we saw high agreement of two methods particularly for the promoters that exhibited large fold changes (|log₂FC|>3). They are highlighted by black circles and fitted by a dashed line. (G-I) Illustrations of top up-/down-regulated promoters in each condition as compared to the growth in glucose minimal medium.

FIG. 7 shows a comparison of Pi-seq to RNA-seq in root colonization assay. (A) Comparison of the promoter activity obtained from Pi-seq (n=4) to the RNA expression obtained from RNA-seq (n=3) across genes in the colonization day 1 samples. There was some extent of correlation for the group II and III libraries (green and magenta points). The group I library was omitted for this experiment because of the low transcriptional activity. (B) Fold changes of promoter activity against RNA expression. The fold changes were calculated by dividing the expression values in the colonization day 1 samples by the values obtained in glucose minimal medium. The x-axis shows the relative promoter activity based on Pi-seq data and the y-axis shows the relative RNA expression based on RNA-seq. Same as liquid culture experiments, there was a high agreement between the two methods particularly for the promoters that exhibited large fold changes (|log₂ FC|>3). They are highlighted by black circles and fitted by a dashed line. (C) Top three up-/down-regulated promoters in the colonization day 1 samples as compared to the growth in glucose minimal medium.

TABLE 1
Reads count mapped to P. Simiae and Arabidopsis
genome from RNA-seq data. Sequencing libraries were prepared
from RNA extracted from the seedling roots in the colonization
day 1 samples (n = 3). The detail of the read mapping
analysis is described in Method section.

| | | Pseudomonas simiae WCS417 | | |
|---|---|---|---|---|
| | | 5 s, 16 s, | | Arabidopsis |
| | Total reads | 23 s rRNA | CDS | thaliana |
| Replicate 1 | 452,547,206 | 26,375,444 | 211,466 | 424,401,754 |
| | | (5.83%) | (0.05%) | (93.78%) |
| Replicate 2 | 397,440,972 | 46,299,996 | 280,460 | 349,387,168 |
| | | (11.65%) | (0.07%) | (87.91%) |
| Replicate 3 | 431,512,824 | 32,709,114 | 214,280 | 397,143,752 |
| | | (7.58%) | (0.05%) | (92.04%) |

TABLE 2
List of promoters that drive putative operons containing 3 or more genes. The left two columns indicate the gene information
of locus tag and function driven by the identified promoter. The third column indicates the predicted number of downstream
genes driven by the respective promoter. The right four columns indicate log₂ fold change and the statistical significance
of promoter activity in day 2 and day 3 colonization with respect to day 1 (n = 4, *adj.p < 0.1, **adj.p < 0.05,
***adj.p < 0.01). The list is ordered in an ascending manner based on the log₂ fold change in day 3.

| | | number | log₂FC in | sig. in | log₂FC in | sig. in |
|---|---|---|---|---|---|---|
| locus tag | putative function | of genes | day 2 | day 2 | day 3 | day 3 |
| PS417_RS13985 | DUF1883 domain-containing protein | 3 | −0.86 | | −1.92 | * |
| PS417_RS01910 | malonate decarboxylase subunit alpha | 9 | −0.42 | | −1.71 | * |
| PS417_RS21070 | efflux RND transporter periplasmic adaptor subunit | 3 | −0.92 | | −1.63 | *** |
| PS417_RS16485 | nicotinamidase | 3 | −0.90 | | −1.54 | ** |
| PS417_RS03090 | 30S ribosomal protein S13 | 5 | −1.15 | ** | −1.36 | *** |
| PS417_RS02280 | dipeptidase | 3 | −1.55 | ** | −1.32 | ** |
| PS417_RS08745 | DUF1446 domain-containing protein | 5 | −0.27 | | −1.29 | ** |
| PS417_RS29150 | hypothetical protein | 3 | −0.67 | | −1.21 | * |
| PS417_RS26170 | imidazoleglycerol-phosphate dehydratase HisB | 6 | −0.71 | | −1.20 | ** |
| PS417_RS24775 | type II 3-dehydroquinate dehydratase | 3 | −0.93 | | −1.18 | ** |
| PS417_RS23630 | ubiquinol-cytochrome c reductase iron-sulfur | 3 | −0.58 | | −1.15 | * |
| | subunit | | | | | |
| PS417_RS10500 | substrate-binding domain-containing protein | 5 | −0.74 | | −1.09 | * |
| PS417_RS05470 | 30S ribosomal protein S16 | 5 | −0.87 | | −1.08 | ** |
| PS417_RS07635 | cytochrome-c oxidase, cbb3-type subunit I | 4 | −0.38 | | −1.06 | * |
| PS417_RS25615 | beta-ketoacyl synthase chain length factor | 20 | 0.75 | | −1.05 | ** |
| PS417_RS06890 | 23S rRNA pseudouridine(955/2504/2580) synthase | 3 | 0.54 | | −1.04 | * |
| | RluC | | | | | |
| PS417_RS13830 | phosphate ABC transporter ATP-binding protein | 3 | 1.43 | * | −0.11 | |
| | PstB | | | | | |
| PS417_RS04040 | 2Fe—2S iron-sulfur cluster-binding protein | 3 | 1.49 | *** | 0.58 | |
| PS417_RS00585 | type VI secretion system tube protein Hcp | 5 | 1.79 | * | 0.61 | |
| PS417_RS09065 | N-acetylglutaminylglutamine amidotransferase | 4 | 1.78 | * | 0.95 | |
| PS417_RS20735 | RNA polymerase sigma factor RpoE | 3 | 1.49 | *** | 1.01 | * |
| PS417_RS06470 | 5-oxoprolinase subunit PxpA | 3 | 0.76 | | 1.34 | * |
| PS417_RS12530 | hypothetical protein | 3 | 1.89 | ** | 1.42 | * |
| PS417_RS17470 | CoA transferase subunit A | 3 | 0.86 | | 1.43 | * |
| PS417_RS18295 | undecaprenyl-phosphate glucose | 11 | 1.00 | | 1.48 | ** |
| | phosphotransferase | | | | | |
| PS417_RS04060 | cytochrome ubiquinol oxidase subunit I | 3 | 1.84 | * | 1.50 | |
| PS417_RS00840 | D-amino acid dehydrogenase | 3 | 1.10 | | 1.56 | * |
| PS417_RS07595 | cadmium-translocating P-type ATPase | 3 | 0.77 | | 1.62 | |
| PS417_RS07020 | hypothetical protein | 3 | 1.80 | *** | 1.68 | *** |
| PS417_RS03190 | endonuclease/exonuclease/phosphatase family | 3 | 1.95 | *** | 1.83 | *** |
| | protein | | | | | |
| PS417_RS01970 | D-amino acid dehydrogenase | 3 | 0.97 | | 1.95 | ** |
| PS417_RS20365 | DUF748 domain-containing protein | 3 | 2.11 | *** | 1.99 | *** |
| PS417_RS19275 | YqjD family protein | 3 | 2.06 | ** | 2.27 | *** |

TABLE 3
List of promoters whose mutations result in colonization difference.

| | | Number | | | | |
|---|---|---|---|---|---|---|
| | locus_tag.refseq | of Genes | product.refseq | logFC.x | Adj.P.Val.x | logFC.y |
| Down | PS417_RS02280 | 3 | dipeptidase | −1.55 | 0.017 | −1.32 |
| | PS417_RS15600 | 1 | Phosphoethanolamine | −0.95 | 0.024 | −1.11 |
| | | | transferase CptA | | | |
| | PS417_RS15740 | 2 | Nitrite/sulfite reductase | −1.75 | 0.068 | −1.53 |
| | PS417_RS18375 | 1 | TonB-dependent | −1.06 | 0.464 | −1.89 |
| | | | siderophore receptor | | | |
| | PS417_RS01095 | 1 | DedA family protein | −1.37 | 0.018 | −1.20 |
| | PS417_RS26170 | 6 | Imidazoleglycerol- | −0.71 | 0.305 | −1.20 |
| | | | phosphate dehydratase | | | |
| | | | HisB | | | |
| | PS417_RS00940 | 1 | hypothetical protein | −0.98 | 0.032 | −1.02 |
| | PS417_RS16485 | 3 | nicotinamidase | −0.90 | 0.249 | −1.54 |
| Up | PS417_RS18295 | 11 | undecaprenyl-phosphate | 1.00 | 0.193 | 1.48 |
| | | | glucose | | | |
| | | | phosphotransferase | | | |
| | PS417_RS13860 | 1 | hypothetical protein | 1.96 | 0.000 | 2.10 |
| | PS417_RS06755 | 1 | DNA-binding protein | 1.10 | 0.032 | 1.27 |
| | PS417_RS12605 | 1 | transketolase | 1.63 | 0.190 | 2.46 |
| | PS417_RS10705 | 1 | GfdT protein | 0.56 | 0.803 | 2.03 |

| | locus_tag.refseq | Adj.P.Val.y | RPL.NRF.mean | RPL.NRF.p | Verify? | overexpress |
|---|---|---|---|---|---|---|
| Down | PS417_RS02280 | 0.036 | −0.69 | 0.000 | Yes | |
| | PS417_RS15600 | 0.007 | −0.65 | 0.000 | Yes | |
| | PS417_RS15740 | 0.074 | 2.40 | 0.000 | Yes | |
| | PS417_RS18375 | 0.078 | 0.52 | 0.000 | Yes | |
| | PS417_RS01095 | 0.032 | −0.83 | 0.001 | Yes | |
| | PS417_RS26170 | 0.036 | 2.30 | 0.002 | Yes | Yes |
| | PS417_RS00940 | 0.019 | 8.55 | 0.000 | Yes | Yes |
| | PS417_RS16485 | 0.027 | −0.64 | 0.003 | Yes | |
| Up | PS417_RS18295 | 0.032 | 0.83 | 0.000 | Yes | |
| | PS417_RS13860 | 0.000 | −0.98 | 0.011 | Yes | |
| | PS417_RS06755 | 0.010 | 2.41 | 0.000 | Yes | |
| | PS417_RS12605 | 0.032 | 0.51 | 0.000 | Yes | |
| | PS417_RS10705 | 0.080 | −1.07 | 0.000 | Yes | |

TABLE 4
List of down-regulated promoters.

| | locus_ |
|---|---|
| | tag. |
| Down-regulated promoters | refseq |
| AATAAGTCGAGCGGGGCGGCAGTGGCGGTCATCGCAGGCTTCTTCCCAGGCAGCTATAAAAGAAGGGCGAAGTAT | PS417_ |
| ACCCGAGCGTCCTGGCTGGCGGATCGCTATAATCGGCGGCTTTGTTTATGAGTGCGGAGTAAGGG (SEQ ID NO: 1) | RS19785 |
| AATTTGCAAGATTCGTCAAAAACCCTGGATTTTGGTCGGATTGTCCTACAAAAACGCTCTATCTCCTGCATTACAGCC | PS417_ |
| TATAGCGCGATCTTAATTTTATTGTTACTTTCCGCAGCGGTGATCACCACGGAGTTCTAATA (SEQ ID NO: 2) | RS08325 |
| ACCGCCGCGTGCTGGAGAACACCCCGCAAACTTGATCAGCCGAGCACTTTTCGGCACTCTACCGAGCTTCGCGGGG | PS417_ |
| ACGACCCCGCGACGCTTCCAGCGCCTAATCCGGGGACGACCCCACCTAACGTTAGGAGGTCATC (SEQ ID NO: 3) | RS18680 |
| ACGCCAAACATGGAAAGGGCGCAGAATCTGGCGTGGAACGCATTGATACAGGTCAGCCATTGCGTTGCAATGGCC | PS417_ |
| CCTCGCTTTCTACATACTTGCGGCCGATTTTACCTATAAAAAAACCTAAACCGTGGAACCTTAGA (SEQ ID NO: 4) | RS07635 |
| ACTTCCACGGACGTCTTGGCCGGCAATTTAGGCCGTGGCGGCCACCCAAGCAAGCGCTTGGCTAAAACTTGTGACT | PS417_ |
| GGCGTTTACAAACCAAGCGCTTGCTTGGTAGTCTCGCCCACAGCCATTCGAGGAAGGATTTCTC (SEQ ID NO: 5) | RS08745 |
| AGCAAGGACGCCGCGCCGGTTGCTCAAGCAACCCGCAGCTGATAGGCCGACAGCGCTAAGACAAACCCCGGACAA | PS417_ |
| TTCCGGGGTTTGTCGTTTCTGCCAGCCCCGCTATCATTCGCTCCATTCTTCGGGGGATTCAACTG (SEQ ID NO: 6) | RS27215 |
| AGGCGTTCGAGGGTCAGGTCTTCAAGGGGAGGGTGTTTAAAGGCTCGCATGAAATGAATCATAACAGGGGGGTAT | PS417_ |
| CGTCGAATACCATAGTTCGATTACCATCGAACTATCGAATCACTCACTCGCTCCTGGAGTTCTCC (SEQ ID NO: 7) | RS17025 |
| AGGGCTGGGCGCAAAGCGCGCAATTAAACCATAACCGAAGGCATGACAATTTATCGCTGGCGGTAGGTAGACACC | PS417_ |
| GTAGTTTGTGGTTAACTTCGACCTCTCGAATTCGTTAAACCCACATCAGCATAAGGATTCCGTTC (SEQ ID NO: 8) | RS15310 |
| CACACCTAACAAGAAAGCATAAGATTAGTTCGCCTTCTCATTTGACAATCATTATCATTCAGCATAATTTGTTGCCCG | PS417_ |
| ATGTGTAGGAGGCCTCAGCAAGGACGCCCTCCCCTGACTTCTTTTGCGACAAGGTGATTTCC ((SEQ ID NO: 9) | RS08760 |
| CACCACTGCCGCCAGGGCAGAGAGCAATTTATAGTCGAACATCAGTTTCTCTAATGAGCGATCAGCAATATTTGTTT | PS417_ |
| TTCTTATACAGCCTGTGTCAGGAGAATAGCCAGCATCGCTTCTCTCTTTATAAGGAACCTCAC (SEQ ID NO: 10) | RS06215 |
| CAGATCCTACGACTTGGTCACATACTTTGCGCTATCCTCGCTAATCGTGCCGCCTACTGCATAGAACTGGACAGTCAC | PS417_ |
| AAAGGCTTTGCGGCATAGTCGCCGGGTTCTGCCGTACCCACATCAATAACAAGGAACAGCCG (SEQ ID NO: 11) | RS24775 |
| CAGGTGACTGAACCAGTGACCGGTGGCCAACCGGGAGCGCCATTGGTCTGCATCCATGTGAACTCCGAAGATTGTC | PS417_ |
| GCCCAGTTGACAGTACGCCACAGGCTGACAGGGCATGATCAAGCATCCTACAGGAGGAATAACA (SEQ ID NO: 12) | RS26745 |
| CCATTAACGGCAATGGCTGGCCGCCTGCGGGACATTGTTTGCCGCAACGGCCACGCTATCCCCAATAAAACCGGGC | PS417_ |
| ACCTGGCACCCGGCACGCTATTTGCTCCAGCTGTGGCACAGAACATTTCCACAGGTGACCGATC (SEQ ID NO: 13) | RS06365 |
| CCATTAATACGGTCGGTAAATGGCCCGAAAACGACCTGCTCATAGCCATCTGAGGCATTCGTGCAATCTGCCCTTGC | PS417_ |
| CGACCCTTTGGACGGCAACCAGAATCCAGGTCAAACCCCGTTCCCTTCACCAGGATAAACGTC (SEQ ID NO: 14) | RS02230 |
| CCCCAATAGCCCTACTCCAACGCGCAAAAACAACCACACAGCAGCAGATGACGTGTCGCACCAGGTCTTTTTTTTCC | PS417_ |
| GCCAAAGAACAGGGATCATCGCCCTCCCCCCACCATCCCCAAAGACGCCGAGGAACCGAGAGC (SEQ ID NO: 15) | RS05110 |
| CCTCGTCGAAAATCTTCAAGGATGTTCGTATTCAGCCGCAACGAAAACAAGTCAATCATCGATAAGTAGTGCAAACG | PS417_ |
| AACAATGACTACAACGAAAGAGCATAGTAGCCTTCCTCAGCGCTTGAACTCAGGAGTCTGTGC (SEQ ID NO: 16) | RS05770 |
| CGCAGGAAATGTTTGGGCATATTCCGCCCGTAGAAGGTTAACGCGACTTTTCATCGCCTTATCCCCGACATCCCCTCT | PS417_ |
| AGGAGCAAATCCTACAGGGGACTACAATCCCCTCCCCCGCGCCATTAGCTGATCCACCCCGC (SEQ ID NO: 17) | RS07260 |
| CGGTGGCACAGGGTAAGGCGTTCGGCCCACCACAATGAACTTGCGACGTTCAAAGACAGCTATAACTCGTTGTAGT | PS417_ |
| TCTTCGTTTTTCTTATAAATCTACAGCTCTGATATGCTTTCCGGCCGCAGGCGGAGATAGCACC (SEQ ID NO: 18) | RS19555 |
| CGTTGAGGGGCAGTCATGGAGGGCGCTCGGTTCGTCAAGGAAAGTTACACTCAGTAGTTTACTTGCTGTGTATTTT | PS417_ |
| GTTGCAATCTTAAAACTACACTGTGCAGTGTAATTAAAGATCGGTCTGTAGGAGTCACCTGTAA (SEQ ID NO: 19) | RS06555 |
| CGTTTGGGAGAGTTTCAATTATTTTTGCCCAGGCATTTCATCGCCGGCCGCAGGTTTGGCTTTATGTTTTGCCGCCAA | PS417_ |
| CCCATGAGAATGCCCGGCACATTCAGGCTGAAACGCTGAATAACAGATCCCGTAGGAAGGTT (SEQ ID NO: 20) | RS06325 |
| CTCCGCGCAGCCTGTCAAGAGTTTACAAACAACCATGAACGTAAGTATATTGCGTAGCAAGCTACTTATCTACTCAG | PS417_ |
| TGTTTGTTTTTTACCCTTCCACCCTTCTTGTGCGCATCCTGCGCGCCTGACCCGAGGATCTTC (SEQ ID NO: 21) | RS21070 |
| CTGGATTGGGGCATTGCGCACTGGGATGCCAGCCGGATCCCGCGCACACTCACGCTGGCCGAAGCGCAATTGAGC | PS417_ |
| TGAAGGCGCTTGCAGCTTGCCCGGCATCCGTTACCATCGCCGCACGTTTTTACACAGAGGCCAAC (SEQ ID NO: 22) | RS29150 |
| CTGTAAGCAATTGCTGTTAATCTTGCGCGCCCAGCGGCATCTCTGACGTAGCGATCGGTTGAACCGGACGTTACAG | PS417_ |
| AGATGCTCTCTGGGCGCGCTCGTTTTTGAGCTTTCGATAATTTCAACAAGCGAGGAGGACCGCC (SEQ ID NO: 23) | RS23615 |
| CTTAGATGGCCTTTCGCTGACTAATGTCCGGTTTTTCTCTGCTTGCTGTACGGTCAGGGAGGGGCCGGTGTAGGATG | PS417_ |
| CGCGCCGTTCGGCGCAGGCCGACACCTCTTTTCGTTCCACACCCTTTGGAACCCGAAATAGCC (SEQ ID NO: 24) | RS21670 |
| GATGGGAGGCTTATTCTAAAAAGGCAGGTCGCTAAACATAAGTTACAAAACCTGTTTAAAACGCCAATACACTTTTC | PS417_ |
| AAAGCATGGTAGTGAAGGCTATAAACCGTGCTTTCGAAAGTCACGCGCAACCGGAGCCTTATC (SEQ ID NO: 25) | RS03905 |
| GCGAGCCAGCCCCAACAGGAGGCTGCATGTTTGTTGGTGCTCTGTTACTCCAACCGTAACGATCCGCCGCCGAGCTT | PS417_ |
| GATTGATGGCCCCCTCAATCACTCCTAAGGTAAGCCCCACGACAGCAAACCCGTGGAGCGACC (SEQ ID NO: 26) | RS01910 |
| GCGCAATGCTTCAGTGGGTAGGCGGGATAAAAACCACGTCACATTCTGTACAAAAAGTCTGCCAGTCGCCTGATTT | PS417_ |
| CAGCGTATGTTTCACCGCAAAATCTTGTAGGGCGATTCCGCCCACTACTTAAAGGGAGTTAAGC (SEQ ID NO: 27) | RS26480 |
| GGCCGATCAACTTCACCCGGCGGTCGGCGCACAGAATAAGTTGCTGGAAAATGTCTGGCCGACGCTAAAACCGCTG | PS417_ |
| TTATGACGCTTTTCTACCGGCAGGCTTTCGGCTAAGGTGGCGCCCCCCCGATTTGGAGCCCCCG (SEQ ID NO: 28) | RS19495 |
| GGCGATGTAGTCGGTGAGTCGAATCAACGTCGAATCCTCTAACGGTCAGAGAAGTTCGAAACGCTAGCATGGACAT | PS417_ |
| ACGACACTTTACTATTTGTCTTTTTGTGGATATITTATCCAGCATCAGACAACGAGGTCCGCTC (SEQ ID NO: 29) | RS15125 |
| GGCGATTTTCGCGAGCAAGCTCGCGCCTGCAGTGATCAAGGCGCTTTTACCCTTTAATCACTCAGATCAATAGTGCT | PS417_ |
| GTTCAGGTTCGCCCACGGTCAATATTATCCACATCATAAACCCCGTCTTCCGTGGAGTGCCCC (SEQ ID NO: 30) | RS04870 |
| GTCAGTCAGCACATGTAGCAGCTGACCCGCCGCCTTCGCGAGCAAGCCCGCTCCCACATTTAGATCGCGTATAGCCT | PS417_ |
| GCCACACCACAAAACCCTATATAATGTGCGACTITTAGCCATCCTGACTCCTAAGGAGCTTTA (SEQ ID NO: 31) | RS04830 |
| GTGAGATTGTCGTCGGCTGTCAGGCCGCCTTCGCAGGCAAGCCAGCTCCCACATTTAGATCGCGGGGCCACAGTGA | PS417_ |
| GATTGTCGTCGGCTGTCAGGCCGCCATCCACTACAATGCCCTCCATTCAACCCCGGGGGGCTTC (SEQ ID NO: 32) | RS10535 |
| GTGGGCATCATCAGGCTCGGCGATCTGAAATTAGGATAAATGTACATAGGTGTCAATAATGGTCGACATGCATGTA | PS417_ |
| CATTTTTATTGCGGGGCCCTGAACTCATCGCTAAGATCGGCCTCACCCCTTAGGAGGACGCCTG (SEQ ID NO: 33) | RS17180 |
| GTGGTTTCTGGTGCTGTGCGAAACCCACCATAGAGAACGGGTAATTCACCCTCAAGGCCTTGAATCCATTCCCTATC | PS417_ |
| AAGCTATCATCCAGGCCCAGCGGGGCTTGATAGTTTTCTATCAAGACGCTCAAGGGGCCTGTC (SEQ ID NO: 34) | RS16920 |
| GTTGTCCACTGAGTTATCCACAGCTTCTGTGGATTGTCCCGAGCGCTTGCTCTAGAACGGGCGTGCACAGTTTTTTC | PS417_ |
| AGCTTTACCTGTACGAAAAAAAGAGTAGAGTGGCGCGCCTTCCGTTCTGTCTCACAGTGCTTT (SEQ ID NO: 35) | RS08345 |
| TCAGGCGCAGTGCGCCGATGCTGCGCTGGGCGATGCGCAACGGTTCAGTGCAACGGGCCCATCGGCGGATGGGGT | PS417_ |
| TGATGTAGGCGACGGCGCATAGCTGTTAAACTTCGCGCCCTCATTTCCCCAGCAAGGCGCGCTCC (SEQ ID NO: 36) | RS04470 |
| TCCTGCACTCAAGGATTAAACGTAGCAGCGCCATATGGATTTAGCACGTGGCCTTAGAGGAACGATTGCAATCATT | PS417_ |
| GCAATTGCAATCATATGTCAGTTTCCGTTAAGGTAATGACATTCGTCGCCAGGTGATCCTCTCC (SEQ ID NO: 37) | RS11030 |
| TCGCGAGCAAGCCCGCTCCTACATTTCGATCGCGACCGACCAGTGGCCCCTTGCGCCTGCCGAATTTACTTGTTAGC | PS417_ |
| TTTTGGCAAAATGTTAACGATAACATTTGCTCTAACAATAAAAACAAAACAGAGAACCACGCC (SEQ ID NO: 38) | RS10500 |
| TCGCGCTGGACTCCATGCAGAAAATTTTCCTACGCATCTGGACAACTGGCCATCATCGACCGTATTGTCTGCCCCGC | PS417_ |
| AGGCCACCGCAGTGGCCGACGTCGCTCGGACGGTTCCGGGCGCTTACGTACTTCGAGGCAACA (SEQ ID NO: 39) | RS06405 |
| TCGCGTCCTGATGAATTGCGATGGTCAGGGCCGGCAGTTTGCCGCATGTCCTCTGTCCAGGCCGCTTTTGGCGGCG | PS417_ |
| TTCGCGACTATAGCAGCAATGATTAAGTGCTTCAATTCCATAAAAAATTGTTATCATCGCCGCC (SEQ ID NO: 40) | RS06890 |
| TGCATGAAGTGGCTCGCCAAATGTGCGGAGTCTAAAGGTACAGCCGCCTGCAGAGAAGGCATGGCTTGACACTCG | PS417_ |
| CGCAATTTACAGAATATTGTTCCGTTACAGAATATTATTCTGTTAACCCATTGCGAGGATAATCC (SEQ ID NO: 41) | RS09430 |
| TGTGATCAAGCCATTCAAGCAAGTCCTGCCTTGATAACGGCCGCGCCGAGCCTAATCGGTTCGGCGTACTCCTAATT | PS417_ |
| GCTCATTATTCTCGGTAACCTGCCGACAACATTCTGAATATTCGTGTGGAATCAGGAGTTCCG (SEQ ID NO: 42) | RS06095 |
| TTCACTTGGCTCTAGATTGCCCTTTCGGCGACACATTTATTTGAAATAGTGCTGTACATTCATCCAGTACTGTCCTACA | PS417_ |
| TTCCATCCCAGGTGACCGGATGTCACCTGAGCTTCACTTTTTAACTATGGATGGATAAAAA (SEQ ID NO: 43) | RS10485 |
| TTCCTGTACGTTCGTACCACTGTTTCAGCCTCGCGACGGTTCAGCATCTTGCAATGGGTATGGCGCCTGCGTAAGTA | PS417_ |
| TCCGCAGGCCGACTCACGAGGTCGCCCTCACCACCAAAAATGACAACAAATCATGAGGCCAAC (SEQ ID NO: 44) | RS23705 |
| TTCGCGAGCAAGCCCGCTCCCCCATTGACCGCGCTTTGCTTCCATCCGCGTTTTGTTCCTCCATCACCCAGATCAATCT | PS417_ |
| TGCTATCCACCCACCATTCGCGCACTTAGACTCAGGCCAATTCCTCCAAAGGTAAGCCGCC (SEQ ID NO: 45) | RS25570 |
| TTTGCCGGACCGAACTGGCTGTTTGCGTCGATCGTGCTTGCATCTGCCCGCCCCGATCAGTACAAAACGCCCTGAGC | PS417_ |
| CGCACGGCAACGCTGGATCCATCGACCGTCGGCCAAACTGCCTGGACCCTGAGGAAGTAAGTA (SEQ ID NO: 46) | RS00530 |
| AACAGCCTTTGTCGCGGATTGACGCTTTCGGCAATTCCCCTGTCGTTTTTGCACCCGGCTCTGTCGGCCCCTGGGCAT | PS417_ |
| ATGCTGGCCCCAAAGCGCCGGCAGACGATTCGGCGCATGAATCGCCAATAAGGGGACGCCTG (SEQ ID NO: 47) | RS02280 |
| ATAACGGCTACAAAAATCTCACAAAGTTTTCACATACCCCTCCCGATACTCCCGCCCGAAGGGCCGCCCCATGCAAG | PS417_ |
| GGTGGCCTCTGTTGCGCGCGCCCCTGCGCGTCAACTGAAAAAATTATCGGGTTGGAGCGCTGG (SEQ ID NO: 48) | RS15600 |
| ATAACGGTTCTCATTAGTCGATTTGGTTATTTGCATATACTCAAACATCACTTTTGCGCATAAACGCAAACTGGTATC | PS417_ |
| TTGCCCCGGCTCCGTTGGGAGTGCGCGGCCGTGCGCGCAGATTTGCCGAGAACAGGACCTAT (SEQ ID NO: 49) | RS15740 |
| CGCAGACCTGAATCTCATAAATTTGTTGCAAGACGTTAATAATAATTACTATCATTCGCGACGGAGTCGTATCCGCG | PS417_ |
| CAGTGAGGACGCGCAACGCTGTTGCCCTTTTCCTCACATAACGGTTCGCCGTCAGGATCGACC (SEQ ID NO: 50) | RS18375 |
| GACAGTTGTGCTTAAGGATGCGACCTGAAGTCGCATTCGTCACTTCTGTAGGAATTGTTCACTTCGTGACACATCTC | PS417_ |
| GATACTTTCATGAATCTTCATGAAGGCGTAGGCTGCGCGTTCATTTCGCCAGCACCCCAGACC (SEQ ID NO: 51) | RS01095 |
| GCACCATCCTGGAGCGCCTGCTTGTATGCACATGTCTATCGCCGACACTCCATCAGTCTTATCCCCTCCGATGTTTCT | PS417_ |
| GTTTGGCGCCCGCGCCCCTATAATGGCTGCCTTTTTCGCCCAATGATTTTGCGGAGTTGGTG (SEQ ID NO: 52) | RS26170 |
| TAAAGTCCTCGCCACCTTGCAGGGTGATGGGGCATAAGCCACTGGTTATCCCGTGGATGTTGATTTTTTTCCGGCAG | PS417_ |
| CCGTCGACAGAAGGTGAAGCTGGGCTATAACTAATCTGCTTTTCGCAAGTCATGAGGTAGAAC (SEQ ID NO: 53) | RS00940 |
| TCCTTCTAGACAGCTGTGGGGCACAGTCTAACGAATCGAGCAGATATGTCATGTACTGGCTCAAGCGTAGATAACC | PS417_ |
| AAATGTTTCTTGCAATGAAAGTACATGACGTGTACTTTTGTTTCCAGGCCACAGGAGAACCGCC (SEQ ID NO: 54) | RS16485 |
| AAAAACAGAACAAAAAATCCAAATGAAACCGTGTCAATAAAAATTTTGCATTTTGAAACACTCTCTCCTAGGTTCTTT | PS417_ |
| CCACTTGCCAGCGAAGCCCGTTGGCAAGCCGTCCTCAAACAACGTCCTCTAGGAGATACACC ((SEQ ID NO: 55) | RS29305 |
| AAACTTCGGTGCCAGAGGGCTCGAGCGAGACGCCCCGCGGGCTGCGGCCATTGACCGACCGGTCATCACCCGGGC | PS417_ |
| GGTTTTCCGTCGACCACCCGGCATTGGTATTTGCCCCACTTCCCCGGGGGCCATAGAATGCGCCG (SEQ ID NO: 56) | RS28260 |
| AAGGTGACGATGAAACACAGGTTCGCGCACACGCTGAAGACCTGGCAAAACTGGTAACTGAAGTTTGCGCCTGAA | PS417_ |
| TTCGGCTTGCCAGTGGTGATGTGGTTGGGTAACATCTGCGCCCACTTTGACCGACGAGGTACAGC (SEQ ID NO: 57) | RS04405 |
| ACCCGATGCCTGGACTCAGGCAACGGGGAGTAACCACCCGATTGCGACCTTTTGCCGTCGAAAACGACACCCGCCG | PS417_ |
| TTGACGCTGCCCCCCGATCGTTATTAAGTGCTATTTATCCGCATTAATACGATAAATCGAACTT (SEQ ID NO: 58) | RS10575 |
| ACGTAAGTGAAGGTGTCACAGAACACTCCTTGAGCGAGACTTAGATACTGATCGATACAAATGTTGCAACTGAGCG | PS417_ |
| CACCGCGTTGTCAACGACTTATGTATCGACTAGTATCTATATCGCTAGCCACCGGAGACTTGAC (SEQ ID NO: 59) | RS09260 |
| AGATCTGAGTGGCACGCTGATAGGCGCCAGAAAAAGACATTTGCAAAAGTCCGGATATTCCTTAGAATATGCGGCC | PS417_ |
| TTTCGGGCACCTATGCCCGCTGTGCATTTAGATTTGCAGCACCGACTACAGGAACGATGTTCAC (SEQ ID NO: 60) | RS05470 |
| AGTTAGGGGTTTATTTCAAAGATGAACTTCGGCTATTTCCCATGGCCCCTGTTATTCCTGAGGGATATTTACCAAGCA | PS417_ |
| ATTGTCAAGTATTTCCTGTACGATGCCTGGCCAAGTTAATTTTCTACAGGTTAGTGGCCGCC (SEQ ID NO: 61) | RS08110 |
| ATAACTCGGTAAATAACTGTITTAAATTGTCGACGTCGTTGAAGACGGGCCGATAATAAACGTGCTGGGGCTGATA | PS417_ |
| AACTCCATCTTAACCTACGTATCGTTCTGATTTTTCAGAAAAAGCCCTTCTGGAGCCCCTACCC (SEQ ID NO: 62) | RS13985 |
| CAATTTCGAGGAAAAAGTACGGTTAAAGCCTTCTGCTGCAAGACTTTAGGCAAATTGACATCTGGATTTATATCAAT | PS417_ |
| ATAGTGGTGCGGGCCCTGCGTGGGGGGTCTGTCTGATGATTTGAAGCATAAATAGGAGGCCAC (SEQ ID NO: 63) | RS19035 |
| CACGGTTTTCTGATGAAAGGAGGTTTTCAGTCGAATACGCGGCTTGCGTGATTAAAAAGGTCTGGTAGAATCCGCG | PS417_ |
| GCCTAATTACGTGCGGTATTCGACAATAGTGTCGAGTGGCGGCACGCTAGCCCGAGGAAGTGCC (SEQ ID NO: 64) | RS25795 |
| CCAATCGCGACACTGTCACGGCAGATGTACTGGCGTGGATCGACCAGGCGCTGACACTGCGCAGGCCGACTCGCT | PS417_ |
| GCGAATAATTTATCGTTTAGAATCAATATTTTAAATTACCGATTAGCCACAGGATGCACCTTTAG (SEQ ID NO: 65) | RS06305 |
| CCCGCAGACAATGACCGTTCGTACCGCCCCCTGAAAAATTTTATAAATTTATCTTGCGCGCAAAACATTAGCGCTATA | PS417_ |
| CATTCACCTCACCAACTACTTAGCGCGCAAACATTTAGCGCTAAAACAAAGAGGAAAACGCC (SEQ ID NO: 66) | RS19415 |
| CCTGGAGGCTGAGCTCCCGGCAATTCCCTTGTCATACGTGGGGCTTTTCATTACCATCCGTCAAAATTTTTATAAGTA | PS417_ |
| AAGATTCAACACTTAGTAGACGCCTGATTTAACAGGCCAAAAAGCTGATGGGAGAGGACTGA (SEQ ID NO: 67) | RS23630 |
| CGAAAGAATGGCGCCCTCAGGGGTGCCATTTTTTTTGTGCCGTTTTTTAAGCATTCTCCGTAACCAAATACATCGCGA | PS417_ |
| TAAGCTGCCAGCCGCCGCCATAGCAGGTAGGATGTACGCCTATTTTACGGGTGGCATGGTTA (SEQ ID NO: 68) | RS01175 |
| CTCCTGTGGCCTGGAAACAAAAGTACACGTCATGTACTTTCATTGCAAGAAACATTTGGTTATCTACGCTTGAGCCA | PS417_ |
| GTACATGACATATCTGCTCGATTCGTTAGACTGTGCCCCACAGCTGTCTAGAAGGACGTCAAC (SEQ ID NO: 69) | RS16490 |
| CTGCCTGGTCCGACTGCCCGGTGGCGGTCGCAGCACCCGGTATCAGGCCAAGACAGCGGGCAAGTGCGCCCCGCT | PS417_ |
| CATTTGCCCCAATCCCCCATGTCTGCTAGTGTCGCGCCGGTTTACCGTCTACCGGAATAGCCGCC (SEQ ID NO: 70) | RS03325 |
| CTGGTCGCAGGGTTGCACCAAGGTCGGTACGCAAGAAATGGGCGACGCAGTAGTCGCCGCGCTGCGGAATCTGTA | PS417_ |
| ATCTCTCGGGCCCGCTTGCAGTTCTTGCTGCAAGGCGGCCCACTTTTTAACAAGGTGTAGTTGCG (SEQ ID NO: 71) | RS09675 |
| GAAGAACGACGGCATAAGGCTCGCAATGGGTCGAATCCGAAGAGGCCCCGAGCTTACACCATGGGGGTGCTCAGG | PS417_ |
| TCTCGCTGCAGCGCATGGAAAAATGTTAATTGACTATGAAGAACGTTCGTTCTACTGTTAGCCAC (SEQ ID NO: 72) | RS14900 |
| GCATCGCGGGCAGCCCAGATGGGTTTATCGCATCAGCAAAATCACCGATATTCCCCGCTCAATCCCCAGCAAAGTTC | PS417_ |
| TGATAACGTGCGTCTATCGATTGCAGCCGGTATGCTTGGCCCGCAACCTCATGGACGCCCGCT (SEQ ID NO: 73) | RS00680 |
| GCCAAGGCTGAGTGTGATCCGCTTGAAGCCCGGCAGCTAGTGCGCTGCCGGGTTGATTATTTGTTATTACAGCGAT | PS417_ |
| ATTATCTCGCGCCCTATTTCTTGGCTTCCGGGGCGTAGGTAGCTGTCAATTGGAGTCCCACTGA (SEQ ID NO: 74) | RS03090 |
| GCCACTATAAAGCGCCGGGAAATATATTTCACCCCGTGACGCTGTTTCAGAATCTTCCTGACCTGCGAAAGCATGCC | PS417_ |
| GTTACGCCGCTGGAACGGTTAGGATAGCCGCCTGTTTCAGTCAGTCTTTTTCGGGAGTAGCCC (SEQ ID NO: 75) | RS22130 |
| GCCGTGAACAATTTTTTACCAATATTTGCTAGAACCCCACGGCACCGGGATAATGCGCACCACTCGGCTATTGCTCC | PS417_ |
| CTGTCCCGGTATTCGGGGCCAAAAGCCGCACATGGAAGCCCGCAGGGGCGGGATAACTTCTCA (SEQ ID NO: 76) | RS25615 |
| GGCGGAAAATGCGTCAGCCGTTGTCGCTGAATATCGCTTCACACCTGATGAAAAAGCACGACTGATGGCGCTTGGA | PS417_ |
| TAGCGTTTCCTGCGCCCTGGCCGCCGCGATTTAGGTAAACTGCCCGCCCTTCGCGGAGGCCAAC (SEQ ID NO: 77) | RS08965 |
| GTAGCCACTTAAGGCTGGTTTTGGAACGGGCCACGGGCCACCTCGAAAAATAAAACGGGTCGTGATTAACTGGGC | PS417_ |
| GGTCCCGGACTCGCTCGGGTAAACTGGCCGCCGCTTTTTACAAGATCAGACGCAGGGGTCAGATT (SEQ ID NO: 78) | RS04380 |
| GTCGGCTCGGTTGAAGCGATCACCGCCAAAGTGCTGCAAGCACTCAGCTGATCGCTCTGCCGGCTACATGGATAAC | PS417_ |
| GGCCCGCTTGCGGGCCGTTGTTGTTTATACTGGCGCACTTTTTTCGACTTGGACACCTACACCG (SEQ ID NO: 79) | RS21760 |
| TGGTGTCGCTGTCCAATCGGATTCTCCTGGAGCAGCAGGTCAGTAGCCGCTAAACCGGCAAACCCTGCTAATGTAC | PS417_ |
| GCCGCCGCTGGCCCGGCTGACCGGGTTGTGGCGGACATTGACCTGACTGTCGGCACGACTTGTT (SEQ ID NO: 80) | RS07375 |
| TGTATCCGTGAAGCTGAATCTCCCTTTATTTAATAGGGTTGTTTCGGCAGGGCGGGCTAACCGTTATACTTTCGCCG | PS417_ |
| CCTGCGAAACCGTGGCCGCAGGCGTGAATAGCCACCATTGAGGGTGAACGCGTGAACCTAATT (SEQ ID NO: 81) | RS00860 |
| TTTGTATTGGACGCTGACAGTGGTGTAGATATAATGCGCCACCTAGAGAGAAAGGTGGGTCCTGCCGTTGCTGCAA | PS417_ |
| GACGACACCGAACCTCCACCCATTAGAAAAAAAAGCCTGTTGAGCCTTGAGTAGGAGATAGCCT (SEQ ID NO: 82) | RS07065 |

TABLE 5
List of up-regulated promoters.

| | locus_ |
|---|---|
| | tag. |
| Up-regulated promoters | refseq |
| AAAATCCATGGACGACGTTACTCCAGGCAATCCAAGTGCCTAACAGACCATGTCTGGTCAGGAATGTCGCTATTTTA | PS417_ |
| TTTGCCGTGCAACCCTGGCACCTTTGCGGCGTCTAACGCCCAACATTACCCAGGAGTTACCCG (SEQ ID NO: 83) | RS00220 |
| AAGATGCGGCTCATGACGGGTCCTATTGGGCAGTCTTCAGGAAGGCAACAAGGGTATGCCTCGTGCCGCGAAGCG | PS417_ |
| TCCAGTCGGAGGAACGTTGCGCCCCTCGCAACAGTCCACCTCCTATACGGACAGAGGACCTCCCC (SEQ ID NO: 84) | RS20365 |
| AAGCCCAGGATCCGGGGTTCAAGTAAGCACACAAGCGGCAACACTTCGGGAACCGAATCTGGCGTTCCGGGTCGA | PS417_ |
| TCAGTGCGCCGGGAGCGCGGTTTATACTCCCGTCGCTCGCCACTTCACCGATTCAGGGTTGATAC (SEQ ID NO: 85) | RS06230 |
| AAGCGCATGCTGGAATCCGTCACCCCCTGACGGCCCCATAAAGACGATAAAGCGAACTTTCCTACGTCACAAACAGT | PS417_ |
| CACTTAGGATAGATGCAGCTCCCGTTGAAACGATTGTTCCCGAACTATTGAGTGGTGATCGAG (SEQ ID NO: 86) | RS05515 |
| AAGCTGGCGCAGATGTATCAAGCTGTTGCGAAAACGCACCAGATTCCGTTTTTGATGTCAGATGTCTCGTCATTCAG | PS417_ |
| TGAAAGCTGAATGCCGCCCTCCCGCGTCGCTGGAGGCGCTCTTCCGCGGTCCAGGAGGCCGCC (SEQ ID NO: 87) | RS20830 |
| AATGCGCCGGGGTGGGTGACGTTTACAGTGGATGATTGAGATGTCACGTGCGCCCAGGCTCACTCATCGCATTGTC | PS417_ |
| AGACAATTTTTTTGAATCTTTTTTGACGTCAAATATCTGTATTTCATGAGCATAAGGAGGTCTC (SEQ ID NO: 88) | RS14340 |
| AATGTCTACGCCAGTTCATTTGACCGCTGAGGCAGTGGGGCACTCGCAGAGATATCCCACACTACCTGAGTCCTTTG | PS417_ |
| CCGTATTGGTTTGCAAGGTGGCGCATGGAAGTCTTGAGGGTGCATTCACTATCAGGATTCAAC (SEQ ID NO: 89) | RS20380 |
| ACAGCGCTGCAGGTGCTCAAGTCCGGGTGATTGGCCAAGAAGGCGTGGTGTTAAGGGTTGAAACTGTTTAGTCAG | PS417_ |
| TGAGGGAACTCGGTCGGGATAACTACAATCATAATGTACAGATAACCCACCCGGAGTCACCCATC (SEQ ID NO: 90) | RS01300 |
| ACCCCCCGCAAAACACCCTGACCACGCAACTCAATATCCGCTGATCAATCAGCTTGAACGCTCAATACCCCGGGAAG | PS417_ |
| TCGTACCCCTGAGACGGCGATTTTCCTGAACGCCGCGGATGTAGACCAACCCGTGAGGTGTTT (SEQ ID NO: 91) | RS04040 |
| ACTCATCCTGATTTCCCACATTCTAACCTCCGCAGGCCGGTGGCGAGAACAGCCGTCATGGCGATTGCACATCAATT | PS417_ |
| GGAACTTATTCTTGTTTGAGAGCGCCTAAAAAGTTCAGGACATTCCCACAATGGAGAACGAAT (SEQ ID NO: 92) | RS15535 |
| ACTTAATACTGACTATTAATACAGGTGACTGTACTTGTGCGGCGCGGGCAAGCACCATGCTTGTCATGCGCCGCCGT | PS417_ |
| TGAACTACCTGGGCTGCGCCCTCATCTACTCTTTCTATTCCCTTCTTTGGTGCGTAATCCGAT (SEQ ID NO: 93) | RS18715 |
| AGAGCCAGATAGGCGAGGGCGCCTTGTTCCGGGTATGCCTGCCGTTGCAGGCCTCGGCATGAATTCAATCGTCACC | PS417_ |
| TCGACGCAACCTCTGCGTGACCCCATGGTCGTAATTGCAGCAACTGACCGGACTAAGGCTTCCC (SEQ ID NO: 94) | RS14190 |
| AGCGAAGCATTGCGTCAGGCACAGATGGGATGATTGCGTGAAGTTTCTTTGACTGTTGTAGGATCGATAACGTCGC | PS417_ |
| GAAGCAGTCCAACACTCAGTAAACTGCCGTTTTTTTCCCGAATCCTTCTTTCCGAGGTTTATGC (SEQ ID NO: 95) | RS14655 |
| AGGCGACATCTTACACTGTAGGTCGAATATTCCAGATTCGGATTTTGCTCAAGGCCGGGCCTAAGCAAAAAAACTG | PS417_ |
| CAACTTTTGCCATCGCCCGGCACTCCATTGATCACTGTCCCATTCACCCGCAAGGAGTCACACA (SEQ ID NO: 96) | RS15520 |
| AGGTTAAACACGTGTAAACCCAGGCCCTTGTTGCACAAATTCTACGAAACGTTGAACCGCCAGAAGCCACTACGGTC | PS417_ |
| AAGTGAATATGGCGCACGCCGAGCCGGCGCGCGCCTCTATTTGAACGTGACCGAGGGCACTCC (SEQ ID NO: 97) | RS14200 |
| ATACGGCGTTAGGCCGGCGCCGCCCTAGAGGGTTCCCACTATTTCGAAACGTTTCACGCTGAACTTTCTCACACACG | PS417_ |
| CTGCGTTCACTTCTTATGCGCTGCCCCGTCCAGGGGCGTTGTACAACAAGGAGCCGCCCCCCT (SEQ ID NO: 98) | RS13380 |
| ATCGCTACACGGCGCAATTGAGTCATGGAAAGCTCCCTGATGGTTGTGGGTGGAATCACCAAGCCTAGCCTAGGCT | PS417_ |
| CAATCGAACGACTGCCACCCTGGGGTGGTCAACCCTTTGAACCCCAGCCTTCGGAGAGCGTTCC (SEQ ID NO: 99) | RS24615 |
| ATGGTTCGTACTCTCCGACGCCTTGCATCCTACTCCGCGCGGCGGACGCTGTGCGCCCACCGCCCGACAAAAGCGCA | PS417_ |
| AGATTGTGCAAGAGCGCGCGATGGGTCGCAGGCCACACTGTGGCTCAATCGATGGAGCCCGCT (SEQ ID NO: 100) | RS04990 |
| CAAACAGGTGGCCAAGCAGTAAATGATGGCGATTTAATGTGATGGCTCCCTGGAGTGTCATCATATAGACATCATT | PS417_ |
| CTGTCGTGAAATGAAGGACCGCAGGCTGCCAGCCTGCGGTCCTTTTTTTTCGATCGAGATCGCC (SEQ ID NO: 101) | RS18600 |
| CAAGCTCAACGGCGAACGCCGCTTTGAGTTCGCCCAACTGCAGACCAACAGCGTGGAAGAAGCCAAGCAAGCCCT | PS417_ |
| GGAGAAAATTCACGATGCCAGTGATGAAATCACCGACATCAATGTGAGCAAGGCGCTGTAAGCCA (SEQ ID | RS13980 |
| NO: 102) | |
| CAAGGTGACCGAGGCGGTATTTGTATTCGTCGCCATCGACGGCAGCGGCCGCACGCGCTCGGTTCCATCGCGCGCA | PS417_ |
| CGTTAAACCTCGCCGGGTTTTGACGGTCAATTGCCCCATTGCCTGCCATGAAGAGTGCTTTGTT (SEQ ID NO: 103) | RS00450 |
| CACCTTGAAAGCCTTGAAAGAACGCGCTGAAAAGTAAGCGCAGTTGATCAGGTGTCTCCTACCTTGGGTGGAACGT | PS417_ |
| TTGCCGAGCCCCCAAGGTCCAGATAGAAGTAAGCCCATACACGGGTTTTATCGAGGAGGCACCG (SEQ ID NO: 104) | RS07115 |
| CAGATCACCGACCCGGCTGTAGCGCCCCATCACCATTGAACGCACCACTCAGTCATTTGGATTGAACGATTATTTGC | PS417_ |
| CAGCCCTTCTCTATCGCAAGACCAGGGGCCGTCGGCAGCCCCGTTTATATGAGGTAAACCAGC (SEQ ID NO: 105) | RS14335 |
| CATTGAAGCCATCACGGCTATTTGCCTTATAACTTATTGGTCTAAAACTCCGCGGATAGCACTGGGTCAGTTTCTGG | PS417_ |
| GTACGCGCCCTTTGGGCGTATTGAGAATGACCCCTCCCCAACGGAAAAACCGGTAAGGAATTT (SEQ ID NO: 106) | RS09065 |
| CCAAGCAGTATTTACTGACGCTGGAAGGGACAGGCCAGGCCGAACTGGTGTTCTAAAAACGCGGATCAAATGTAA | PS417_ |
| AAAGAAATTTCAAAGCCACCGTGCGGTCCATTATTTCATCACCCCCTTACAAAGGAAAAACGGTC (SEQ ID NO: 107) | RS20890 |
| CCCAGCATGCGGCGTTGCGCAAAAAGGGCTGACGGCGGCAAAAACTACTACTTTAGTACTGGTTTTCCTGTCTATCG | PS417_ |
| TCGCATTCCCAAGGGCTCGGGACTCACGTGTAATGCGCGCATAACGATAAGGATCGCCCTGCC (SEQ ID NO: 108) | RS10720 |
| CCCCCATGATTCAACATTTTAATAATTAGGTTAGAACCAATCGGCATTTTACAGCCCTGGCTTCGGCCCCTACTTTAG | PS417_ |
| AGGCGAATAACCGACCCCCTGCCCGGTGGAGGTGTCCATCACGCACTGAACCAGGACAGACC (SEQ ID NO: 109) | RS12175 |
| CCCCCTGTTTATGGCTGACTGTGATTTTTGGCTGAAAGTTTCCAGCGTCCTACAGACGGCATAAAGATGTCGCTGAA | PS417_ |
| GGGGAACGATCAACGCCCAAACCCGTCAGTTCCTTACAGACCCTTCTGAAGGAGCCCGGTGTT (SEQ ID NO: 110) | RS03195 |
| CCCGATGGCGGCATATCCAGCTGAACTTTTAACCGGAGCCCAGGCTCGGACCTATTACGGTCATGCTCAGGAAGAG | PS417_ |
| CGCGGCCGCCTCAGAATTCGGATCAAGCAAAGAGCGTCCACAGGGCGTAAGGGGAAGATGCTCG (SEQ ID | RS15255 |
| NO: 111) | |
| CCTGGCGGCCGCAAGGCCAGATTCCCGACGGGGTGGGTTAAAAAAAGAAACGAATTGAAATTCGCCAGACTCCAT | PS417_ |
| ATCCGTGGGGGGCAGTGGGTGCAGGCTCCCGCCAGCAGCCGGTATTACCTGACAGAGGAAGACTT (SEQ ID | RS04060 |
| NO: 112) | |
| CGACGAAATAACGAATTTGGCGCAAGGTCAGGGACATAGCGATATCTGTTTTTCAGAAGATGGGTCATCTGATAAT | PS417_ |
| AGATCTTCCCAAGGGGTCAAGCGTCAGCCTACTTTTCCTCCATCACTTAGGCGGAGCGACTCTG (SEQ ID NO: 113) | RS06470 |
| CGCCTGAAAGATCTCTATACGGCACGGCCCTTGCTGTACAGCTTATGAGGGAAACGGAATTTGGCCGATGGTCAGG | PS417_ |
| AGACGCGAACTAATCCCGACACCGGTCGTCAATGACTGCATGTTCTTATTAGGCGGTACGCACC (SEQ ID NO: 114) | RS22725 |
| CGCGCCTAAATTATTAGCGCTTCAAACTGGCTGCTTTGTTCCAGTCGGGTTCTTCGTTTGATCAATGGTGATCAACGT | PS417_ |
| TACTGGAACGTTTTAACGTTGCACGGTTCTTAATCCGTGTCATTTGTAGGAACACCAATAAT (SEQ ID NO: 115) | RS08335 |
| CGTTTGCCCGGCATTACGACCCGTAGGAAGAGTCTGAAAATATCAGCGAATAGTATTTTTATTCCGTAAGTAAACTA | PS417_ |
| CCCCGCTCCGTGACGGTCTACAGAACCGCAAATAGAGTGTTTATCTCCAGAGGTTCTAAACAG (SEQ ID NO: 116) | RS15770 |
| CTACAGCGCGTTTCGCCAGCACACCGGCCAGTCGCCCAAGGCCTACGTCAAACAAATTTCCCTGCGTGCACGCGCGC | PS417_ |
| AAGACAGCCCCTGACACCGCGCTCTAGGATCCAACCTATCGAAACGAGGTAGGGGATCCCGGC (SEQ ID NO: 117) | RS14630 |
| CTGACGCCCGGTAAATAACCGGATCTACATGTGGGAGTGGCTTGCTGTGGTGAGCAAGCCCGATCACTGCAAATAG | PS417_ |
| TTAATCGAACTTCTCCTCTGCCAACCGCCTCCTACCTGATAAGCCCATCGCCCCCGGCACGCCC (SEQ ID NO: 118) | RS21405 |
| CTGCAACTGCACCCTGGCCAGAGGCTGTGGGCACAGATCAAGGCCGTCGCAGTCTTGGCGTAAGCAACCTTTTGGC | PS417_ |
| ACGACCGCACACCGCTGCGGTCCATCAGACATACCGGCCTGATTTTTGCCAAGGAACCTGCCCC (SEQ ID NO: 119) | RS13950 |
| CTGTCGAGTTGGTCAGAAATTGAGGTTTTTCCAGGCAAATCGGGGATTGGGTTAAACCCCCAGGCGGTTTGCCAGT | PS417_ |
| CGGAGTTCTATAAACTGTCTTAAAAGGCTGTCACCCAGCCAGTCGTATTCAGGAGTCCTGTGCA (SEQ ID NO: 120) | RS25020 |
| CTTTAAAAACAGAAAAATTGCGCCGCGGCCCGCACGACGATCCAGACCTTATTTGTAGCTGGCCCAAGACTTGCCAA | PS417_ |
| CAAACGACGATTAGGCGAGATATCATTCCGTTTATTCATGCAAAAAATAAAAAGAGGCGTGCC (SEQ ID NO: 121) | RS25110 |
| GAACTAAGCTGACCCCGTTTCGTTCTTCTGTAGGAGCGAGGGGGACCCCTGGTCCTTGCTCGCGAAAAAGGTACAT | PS417_ |
| CCGACACCGTGATGCGCCAGGAATATTTTCGCGAGCAAGCTCGCTCCTACAAAGGGCTGCACCC (SEQ ID NO: 122) | RS08120 |
| GAACTGACGGGTTTGGGCGTTGATCGTTCCCCTTCAGCGACATCTTTATGCCGTCTGTAGGACGCTGGAAACTTTCA | PS417_ |
| GCCAAAAATCACAGTCAGCCATAAACAGGGGGCCAGGCCCCCACTTCTTCCACAGGAGCAATC (SEQ ID NO: 123) | RS03190 |
| GAGGGCATATACCCGAGATTCAATAGTGGGACCGGAAATTTCCTACTGAATCACTCGGTTTCTCCGACAAGATGAA | PS417_ |
| CTTTGCCACGCCGTCTATCCTCAGAACCCTTACTGACGCAAAACCACCGTTCGAGGAGACCGCT (SEQ ID NO: 124) | RS27475 |
| GAGTTGTGCGCCGAACTGGGCATCAAGGGCGAGCCGTCCCTGGAAGCGCTGAAGATCGCCGTTCACGTCTGAAAT | PS417_ |
| AAAACGGAACCCCCACCAGGGCTGGCTATCCTACAAGTGCATGCCCTCACTTTAGGAGCGACACC (SEQ ID | RS09035 |
| NO: 125) | |
| GATAAAAAGCGGGAAAAAACGCTGGGCATTAAAGCATTTTTCGACTTGGGGCGTCATTCGCACCTGTTCAACAAGT | PS417_ |
| CGACCGAATGCATGGCGCCCAGCGGCAATCAGGCGGTATGACAGACAAAGCTTTAGGGAGTTTT (SEQ ID | RS25775 |
| NO: 126) | |
| GATCAACAAACGCATCGGCAACGAACGGCTGGAGAAATACGACTCCGCGCCCAACGGTCAGGAACAGGGTTGCCC | PS417_ |
| TTGACGGGTCTGATCAACTAACCGACAGCAGCGGCGGCCTCCGGGTCGCGCTGTGAGGATTGGCA (SEQ ID | RS02110 |
| NO: 127) | |
| GCAGTTGGCGGCGCTGGCCAAGATTATCCATGAGAACGGCGGCGGGCGCGGCCTGATATCCATCTGCGCGGCGGG | PS417_ |
| CGGGCTGGGCATTACCGCCATCGTCGAAAAGTAGCTCTATCAAAAACAACAACAAGGAGACTGCC (SEQ ID | RS12990 |
| NO: 128) | |
| GCATGGGGGCAGCCAGTTGACTTGGGTTAACTTATCGACGCCGTAAAAATTCATCCAGCGCGGTTGAGCTGCCGAC | PS417_ |
| ACTGGGAACGACTCGCAGTAGCTGTCAGTCCAAAGTTACCTACTCATTAGTAAGGTACAGCGCG (SEQ ID NO: 129) | RS14365 |
| GCCACCGTCAACGTGGTGCTCTATGACCGCTTGGCCAAGGGCAACAACACCCGTTCGGGCCCCAAATACTGATATTT | PS417_ |
| TCGGAACATCCGGCACCTGCGGGCAGTCAGCTATTTATCACTAGCCCTCGTTGGAGACAGATC (SEQ ID NO: 130) | RS20455 |
| GCCACGGCTCGCACCATTTTCAGTTGGGCCAGTTCCATTGCTCAGTTCCAGTGATTTCAGAAGTCAGAAACGGTTAA | PS417_ |
| TTGTCCTACGCGTTGCTCGGGCCAAGACTGACAGCATTCCTTCTCAGTATTCGGATGCCGTCG (SEQ ID NO: 131) | RS07535 |
| GCCGAAGTTCAGCTCGCTCAACGTGACGTGGGTGGAGAGCAGCATGAATTCCCATCGGTTCGCAATTGACCCAATC | PS417_ |
| ACATTGAACCGGTGACCCGCCACAGTCCTCCAAGCTGAATAGACCACGGCCCGGTACACCTTCG (SEQ ID NO: 132) | RS14520 |
| GCCTGCGCTTATGGGCTGGCCTTGATCATCTCGTGCCTGGTGGTGGCTCGCTTCCGTCGCGGCGTTAAGCCCGGCG | PS417_ |
| GGCAGGCCCTGTCAGCCCAATCCGCCTGAACGACGCCACCCAAACCGATCAATGAGATAACGCG (SEQ ID NO: 133) | RS10070 |
| GCCTGGAAGATTTCGATATGACGCAAGCGCATGGCAAGTCCATAACCTTAGTTTATAGGTGATCCATCTTTATTCATT | PS417_ |
| GTCGGCAGCCTGTCACCTGCCCCTAGGCTTGAGGCCTCTGGATTTAAAGGTGGGCGGCGGAA (SEQ ID NO: 134) | RS01970 |
| GCGCGCATGATAGCCGAGTTCGGAACCTTGGACTTAACGATAGAGTCCAGTTATTAGCCTGCTAATGAAAACAGGG | PS417_ |
| CTAAGTTGATAGCTCTGTTCGTCCCGTCAGGTCCGTAGCCGTGCGGACCATAAGGAGTCGTGTC (SEQ ID NO: 135) | RS20350 |
| GCGGCCCTGGATAGTTCAAGGAAATTGCCCGCGCCAACCCGGTGCAAATTTGCTGAAAATTAATTTAAACCTTTGCC | PS417_ |
| ACCCGACCCGGATCAACTCTCCCATATCCCATCCTTCGTACACCCATCAGGAGACATTCGATC (SEQ ID NO: 136) | RS12525 |
| GCGGCGGGTAAAGTTTCTCCGGTCAGATGCGCGGACAGATCGTGTTACACCTGATGAGAAACTATTTAGTGTCGAA | PS417_ |
| AGCCGAACCCAAGGCGTAGCGTGTTATCGAAAGCACGTACTCCTCGCCCCAGAGAGGCTTCGTC (SEQ ID NO: 137) | RS14915 |
| GCGTTTTTCGTGTGTGCCAAATCAGTGCGTGCCGGAAACTTGCTTGAAGGGGGAGAACTTTTGCGAAAAGACCGAG | PS417_ |
| TCTATGTTTGCAAGCCTGATCGTTTAGTTATGCAAGCCTCCTTAAAGTACAACGAGGAGTGTTC (SEQ ID NO: 138) | RS20735 |
| GCTACGCCTTGGGTTCGGCTTTCGACACTAAATAGTTTCTCATCAGGTGTAACACGATCTGTCCGCGCATCTGACCG | PS417_ |
| GAGAAACTTTACCCGCCGCTTTCGAGTCACCAGAACACAGCCTCTTCGTAAGGAATCGCACTT (SEQ ID NO: 139) | RS14920 |
| GCTGCAAATATGCAGAAAGTAGCGCTGGGGCAGTACCAAGGTAGTAGCCCCGCGCAGCGAGTGCGGTCTTAGCAT | PS417_ |
| GGATCCCTAACGGTCTTATTCACAGGAGTGACACGAGCATGCGCGTTTTCACCACCCGGCCCGGC (SEQ ID | RS10745 |
| NO: 140) | |
| GCTGGTAATGATCGTGCTGGTCGTGAATATCGTGAGCCGTATCTTCGGCAAGCCCAAGTACTAAGAACGGGAGCAC | PS417_ |
| CTGAATTTTGAACGTATCAACTGCGCAAAAAGCCGCTCCGTTTGTCACCCAGGCGCCTGTGGTC (SEQ ID NO: 141) | RS13830 |
| GGAACGCTAGACTCATCTGATCTGCGTCAACTCCCTGTCGCTTGAAACTCCCAGAATCCACCCCATCTATAAGGGTAT | PS417_ |
| CTGGCAGGTTTCTTCATTCAGGATGTTTTATAGCTCCGGCCTGCTCAAAAGGAAGGTGACTA (SEQ ID NO: 142) | RS23950 |
| GGACGACCGGGATCGCGAATGGCAACAGGAGAAAGACCGCGACGCAGGCCGGACCTATCGCTCGCACTAGGTCG | PS417_ |
| CTTTCAGACATCCATCGAAACTAAAGTGCGGCGACCATGGCCCAAACTCCATAGAGGGAGAATGTC (SEQ ID | RS04150 |
| NO: 143) | |
| GGCCAAGGCCAATGGGGTGACATGCCCGCACCGGCCTTGCCCACGGGCGCACGCCGGGAAATCCCCACCTGGCCA | PS417_ |
| AAAAAGCCCTAGGCATTCGCCCCCGACCTGACGCGGGGGGGGATGCCCGCCCCAACGCGGGGCAA (SEQ ID | RS15675 |
| NO: 144) | |
| GGCGGGTGCGAGGCAGATCAGACCACCGGGGTTTACAGCAGACGATTGCACGTAGGTGTCGGTCGATACCTTTGT | PS417_ |
| ATGACTTGCCAGCTGGGTCGCTCGATGCTGCCATCCACCCTCGATAAACACGGGACCCAAAGCCA (SEQ ID | RS17650 |
| NO: 145) | |
| GGGAAAAACCTTACATGACTATCGTCCAAAACCCGTAGAACGCCCACTAGGCAAACTTTCCGACGAAGTGCACATT | PS417_ |
| GAACGCCCTTCCGCAGATGTCTGACGAGTGTGCTGGGAGATTGATTAGAACTGCTGAGAACCGT (SEQ ID NO: 146) | RS02475 |
| GGGCGTTGTGCGAAGCTTTGGTGCGTGAAAAATTCGTGAAATTCCGGTGTTATCCACTGGAACTTCTGACTACACGC | PS417_ |
| CAGCTTCTATAGCGAGATAGCCTGTACAAAACCATATAACTTCGTTCGGAAAGGATGTTTCTT (SEQ ID NO: 147) | RS24145 |
| GGGCTTGCCCGCGATGCAGGCGACGCGGTCTGCCAGCCCGACCGCGGGGATGCTATCGCAGGCAAGCCAGCTCCC | PS417_ |
| ACAGAAAAGCACTGCCCCATTGGCGGTAACCCAATATCGTGGCTGAGACCACACAGGGAGAAAAC (SEQ ID | RS00585 |
| NO: 148) | |
| GGTCGTTACCAGGGATTACGTGACGTAGCACTCGAATATGGCTTTGTGTTCAAGGCGCTGGGGCTGGTTTAAGGAA | PS417_ |
| CTTAGCGGGGCGGTTCTGTCTGAACACAGGACTGCCTACTGATTGATGGATCAAGATTGCTGCC (SEQ ID NO: 149) | RS20425 |
| GTAGACGCCTTCGCAGGCAAGCCAGCGCCCACATTTGATTGCGCCCAGTCTGTGAAGGGGGCTCATTCCAATGCAA | PS417_ |
| CGAATGGCCCACACCACCCTCATATGTTCACGAGCAATGCTTTACGACAACAGGTACCAGACTT (SEQ ID NO: 150) | RS13800 |
| GTCGTAAAGCAAGCGTAGCAGTCAATGTGCGTACTGCTTCTGGCGGCCGTTTCTACCTGAAAGTTCATATTTACTGC | PS417_ |
| ACTTTCTGAAACGGCCGCAGTCGGTATCTACACACGCCACTCAATCACTTCAGGAGAGTCCAC (SEQ ID NO: 151) | RS27495 |
| GTCTAAAAATAGGACAGATAGTTCCCACAGGGACCACCGGAACCATCACAAGAAGTATCACACCTTTTTTGACACTG | PS417_ |
| CCATTTTGCGCCTCCCACGAGGACGCGCTTCGGATATTAAGACCTTAGCGGACCCTACAAGCC (SEQ ID NO: 152) | RS05830 |
| GTGCCGCCAGCATCAATTTATGACAAGACTTTAAACCTTGTGAGTTGCGGCTAGTCTCAAGGTTAAGTCGTCGCGTT | PS417_ |
| TATCCACGCAGGCGACTGGACACTCATCACCGCCTGCTAAACGCGGGCTTCATTGAGGTTGCC (SEQ ID NO: 153) | RS21775 |
| GTGCTGCCGGAGCAGCCATTGCCGAGCTACCTGCACAAAATCAGCTGAATTCATGCAATTTGTTGCTAACTTCACCC | PS417_ |
| GACCATTGAAACCACCGCTACCCCGCCCCATCTAACTTGCATACTTCCTTATGCAGGTGCCCC (SEQ ID NO: 154) | RS22550 |
| TAAGCAATTGGGTACAGACCTTACACAACCTTTACCTAGCGGCTGTGCTGTTTTTCAGAAATACGTTGTCTTAAAAGT | PS417_ |
| ATCCCCGCGACATTACTTGAGTCTATTGATTGCAGACCACAAGCGCTGGGGCTAACCGAGGA (SEQ ID NO: 155) | RS17580 |
| TAATGTTGATCCGCGAAATCGAGGGTCCGGACAAAAGTTCCGGTTAGGCAGCGATTTATATATCTATGGAATAAAA | PS417_ |
| GGAACTAATGCCGAACCGCCATGCTCGAAAGGCCAGTAGATATCTTCAATAAGGAGAAACACCC (SEQ ID NO: 156) | RS18980 |
| TACCAGAGTGCTGGCTGAGCCGTTTCAGCCCATGATCAAGTGCAAAAGATTCAACCTTGCGACATTCTCTTAACAGA | PS417_ |
| TCGACAAGCCACGCACCGTTCCGTCGCACTTAATCGGAGCGCACGCACATTAATGGAGCAGAC (SEQ ID NO: 157) | RS22895 |
| TACCGCGCGATGTCGTTGAGGTGGCGCAGTATCCTGCAACGACATTGTTATGACCACCCATCGCCTCGAACACATCC | PS417_ |
| AATCGAACCTATCCAGACGAGTTCTACTCCACCTGTATGAACCCTGATGGTGGAGTAAGAATC (SEQ ID NO: 158) | RS05085 |
| TCAAAGGTCATAAAAAAGGAACGGCCCAAAACCCCATCAGTCACTACTTAAGCAACTCGATTTCCCAGGTGCCGTGC | PS417_ |
| CTGGGGTTTTGCTCAAACGAGCCGGGGACCGCTCTGGCCACTTTGAATAAGGAGTTTTGTCTG (SEQ ID NO: 159) | RS27325 |
| TCAAGCCACCAAGCGTCCATCGAAGCTGTCCTTTGTCATGGTTGGTAAAGGATGGACAGCGGTGACCGCCAGTCAG | PS417_ |
| TTGCCTGGAACCCCCACACATTGCTGCCCCCTAATCCTCATAGCCCATGAATAAGGAGAACGGC (SEQ ID NO: 160) | RS15265 |
| TCACCCGCAACGGCACCCGGTGGTGGTGACGAGGCCACAGGCGCAGCGTTCACCATTCGTCTAATGCCCGGCACTC | PS417_ |
| TGAAGAATGTACTGTCTCTGAATAATAAGCACGGATACCCCGAGCGTAAGAAAGGAGAGCCCGT (SEQ ID | RS13850 |
| NO: 161) | |
| TCAGGTTGAGTCGTGTAGTGGTTGGAAAACCTGGCGCCGTACACGTTCAAAACGGTTTCGCTTGAACATTGATTGAT | PS417_ |
| GTGAAAGGTCTTTTTTTGGAACGGGATTGAGAGCGCAATCGCTCGTGCGCAGGAGCAATTATC (SEQ ID NO: 162) | RS14325 |
| TCATAAGGCTGTTCCTGGCAAGTCTTGAAGTCATTTCATGGACTCTGGTATGCCGATTGGGTTCCTCGACCGACCTG | PS417_ |
| GAACAATGTGCCGGATGCCCAGTCAAACGCCTGCTTTCACCCCTATACCCAGAGGTTGTTTCG(SEQ ID NO: 163) | RS02375 |
| TCATCGAAACAACCTCTGGGTATAGGGGTGAAAGCAGGCGTTTGACTGGGCATCCGGCACATTGTTCCAGGTCGGT | PS417_ |
| CGAGGAACCCAATCGGCATACCAGAGTCCATGAAATGACTTCAAGACTTGCCAGGAACAGCCTT(SEQ ID NO: 164) | RS02370 |
| TCCAGGCTGACGGGCATAACTTGCCTCTGATTGTTGTTTTTAGCGGATTTTCATCCCGTGGTGCGGGAATTAGCGGC | PS417_ |
| AAATATCGCAAAGTTCATCTTCAATTATGAGCATAGCATTGTAGGAAATAAGGAGCGCCCGAC (SEQ ID NO: 165) | RS16290 |
| TCGCCTGGCCAACCTTAATACGCCGGAGCTGTTACAGACGCCATCGGGCGTGTCAGAATGAACCTCGCACAAGGAA | PS417_ |
| CTTTGTCGGCGCTGCACGCGTCACAAGGTCAGCTATTAAAAAGTATTCTCTCGGAGACAGACTC (SEQ ID NO: 166) | RS19245 |
| TGAAAAGGGTTTTTGCCGAGAATTGTCGCCAAATATAGGCAGTTATATAGTGAAATGCACTGGCAATTGTTTTTTAC | PS417_ |
| ACTGCGCTCATCATTGCTCGAATAACACACGTCAGCGGCCATGCCCGCGATGAGGATATAAAA (SEQ ID NO: 167) | RS00840 |
| TGAAAGCGCCGCGTATTCGGCGGGTATGCCACTTTTTTAAGCGTCGTAGCAGTGCTTAACTGAAAAAACCGGCCTCA | PS417_ |
| ATCGACGGGCACCGTGCAGGAGCGAGCTTCGATCGAAATGGGCCTTTTTCCAGGAGGCCCGTC (SEQ ID NO: 168) | RS03830 |
| TGAACGACCGCAATTGTTTACCTTTGATAGCTTGCAGTGTCGACCTGTGCGGCCGCAGCCTCTGTTATTCAAGGCGC | PS417_ |
| CCGGCACATTCCGCTGAAACATTTGCTGGTCTGTACAATCACACTTAAAAAGGAGCCTTAACA (SEQ ID NO: 169) | RS09155 |
| TGATCCTGCAAAAAAAACAGAACCCCTGCGTGCCGGCGTAGTCATCTGCATAGGCCTCGACAAACGAGGCTCATCA | PS417_ |
| GTGATGTCGCTGGCAGCCCCTGAACGCCAGCAGCGTCGAGCCCTTTTGCGCAAGGAGATACCCT (SEQ ID NO: 170) | RS04290 |
| TGGATGCAGGAGGAAGTCAGGCTGTCCGGTCACGCCTTGTAAAATCCGATAAAACTTTTTGTGTTGCGCGGGACTC | PS417_ |
| ACAACTTGGAGCGATCACGCCGGCAGAGCGTGGCGCAAAACCGGATTAGTACCAGGAGATTGAG (SEQ ID | RS25015 |
| NO: 171) | |
| TGGCAGGGATATTCAGTGGAGGTCGCCGATACCCGGAAAGTTCACGGGAATGACGCTCGTCTCCTGGGCTGAACG | PS417_ |
| GCCAGTGATGCGGCGGGTCACACCTTTAACGGGCTACCTTCCATCCGCCCGCAGGAGAAATCAGG (SEQ ID | RS08075 |
| NO: 172) | |
| TGGCGACTGAACCTGTCGTGCAGGTTGCATGGTCGGTGCGGTTGCACGTTGCATTCCACAACCCCGGGACATTCGC | PS417_ |
| GCAAATTTTAATGAACCCTCGCGCTAAACACTTGACCTAACGTGTACGACCTAGGAGGTGAAGC (SEQ ID NO: 173) | RS12520 |
| TGGCGAGCCTGCGGCTTGCCGTCCCCGCTCAAATGCACCAGGTCATCTAGTGCATTCGTACCCCGTATTGCGGATTT | PS417_ |
| TCTTTGCACTATCCTGAGGTGCGGATGTCGTACTTTCCATGCGCCCAAAACCCCGGAGATCTC (SEQ ID NO: 174) | RS17390 |
| TTTGGCAACAAGCCCGCTGGATTTGACGATGACCGTTCGTCGCACTGGCGAGTTTTTTCTAAACCTTTGCCGCCCTG | PS417_ |
| AAAATTCGAATTGAGGGGGGGGTGTGTTCCCCCGCCCTTATTTTTAGCCTGGAGGAACCCATC (SEQ ID NO: 175) | RS20895 |
| TCAAATGGATAGCTGCATTTGCAATGAAAAGTTGCGTATTACCCAGAGTAGCCAGTTTGTCCAGCGCGCTAAAGCA | PS417_ |
| GCCTGCTGCTTTATTGAACAAACAATCAAACAGCGAAGTCTGGAGTGCAATGAGGGATGTTTTT (SEQ ID NO: 176) | RS18295 |
| TGACGCCAAGGAAATGTCAAATCTGAGGCATGATGCGGCCTCTTTGCAATTCAGGTTGAACTTTGGTCGTGAGCCTT | PS417_ |
| GTCTTAACACTCATCTTGCGGCCAATTCCAAAAACCGCTCCCCTGAACTAACCGGTTAAATAT (SEQ ID NO: 177) | RS06755 |
| TTAGAAGCTAGACCATTGCGAGGGGTCTTGTCATGTCCGGGGTCAATACAGCCCCGTTCGTCGCAGCGCGGGCGTT | PS417_ |
| TGGATCAAACCTTTGCCGCCCCCTGCCCTCCACTCTAGATAGCCAACCGAGGGAGACCGGTTTC (SEQ ID NO: 178) | RS12605 |
| TGAACAGGCCTGTCGCCCAAACCGAACGCTGCCTGGTTGTTGGGGTCCTAACCCTAGGTGTCGAGGTTTTTACCGCC | PS417_ |
| CCGCCCTCCCCGCGCGCATTGAGGGTCTAGCGATGTGCGTACCCCCGAGAAGGAGACGCTTAC (SEQ ID NO: 179) | RS13860 |
| TTTGAACGTAATCGTTCCTACGCGGCGTGCGGGAACGATCAAGCGCCCTCATCCCATCGCATCCCCCTACCCAGTAC | PS417_ |
| CCAAGTAGCATAGGTCCCAACCCCCGCTCTTCGTAAACTCATGCCTCAGAGGGGGAGGCAACG (SEQ ID NO: 180) | RS10705 |
| CACCGAAGTAGTTAATGTCAGATTAATGTGGTTTCGTTTGTATTGACCGTATTGGTGGTTTAGGGAACTTGTCCTAC | PS417_ |
| GGTTCCCGGTCAATTTTCGCAGTGCTTCACTGCACGAGTTATGTGAGTCGGAGGTTTTTTCGT (SEQ ID NO: 181) | RS01735 |
| CCTGCGATGGCTCGGCGGAACGAGCCATCGCTGACGTCGATAGTCACCATCAGCCCAATGAAGTTCTCTTAAAAAA | PS417_ |
| TGCAACGCGTAACATCAACCCCATACCAGCAACAACCCAACTAAAACACTCGGAGCACACCCTC (SEQ ID NO: 182) | RS12055 |
| CTATGGGTCAGCAGTGACATGGCGCAATCCTTCGGCACGGGATAAAGGCGACACTATGCCGCGCCAACGAGCAGA | PS417_ |
| TTGTCATTITGAATGAACCTTTTAGAACGCTGCCCTATCCATCTATAACAACACAGGGAGGCAAC (SEQ ID NO: 183) | RS11540 |
| CTGTCATGACGCGACTCCAAACGGTTCGGCGACTATGCTGTTTTTTAAATCAATCAACCTTAAAAAACAACTTAACAA | PS417_ |
| ATCAATCAGCCGGAGCAACACTCGGTTCATTGGCTGGCCCACAAACAATAAAAGAGGTGCAT (SEQ ID NO: 184) | RS17470 |
| GAGTTGCAGCGCATTTTCGCGGTTACCGGCACACAAAATCTTACAGGTGACACAAGACCGTTCGTCGCCATCAAGG | PS417_ |
| CACGAACACGAAACGAGGGCTGACCAATAGTAGGTAAGGACATTAATCATATGGAGACACCCTC (SEQ ID | RS07020 |
| NO: 185) | |
| GCCAGCGCGGCGTCACCCTCCCACGCGCTTGATCGAAAGCTCCTACGCGCAAAGGGAATTAGTCTGCATAAAGTGA | PS417_ |
| CTCTATCTAGGTGTACTGCCTCGGCACGTGTAAAACGCGCCGAGCCACGCTTGAGGAGTACCTT (SEQ ID NO: 186) | RS21450 |
| GCGGCCACCGCCGCAAAAGAATCCGCCAAGGCCCTGAAGGCCGCCGTGGATTCCGGGGCACTCAATTCCTGATAAT | PS417_ |
| CCGATAGAACTCTGCTGCACCACAACCCTCCCATGCATAACGACCACCCTGCATGGAGAACGAA (SEQ ID NO: 187) | RS16110 |
| GCGGGCGGATGGAAGGTAGCCCGTTAAAGGTGTGACCCGCCGCATCACTGGCCGTTCAGCCCAGGAGACGAGCGT | PS417_ |
| CATTCCCGTGAACTTTCCGGGTATCGGCGACCTCCACTGAATATCCCTGCCAAGGAGGTCCACCC (SEQ ID NO: 188) | RS08070 |
| GGGCGCGGGTCCAGCTGGCGAGGGAAGTGGGTTCGGTCATGGCGATTGGCGCTTGCGGTCAACAGGTTGGCGTG | PS417_ |
| TGGAGCTGATGCCCTGGCGGAACGCTTGAGCCAGGATGCAGCTATCAATCACCAGAGGATGGAAGC (SEQ ID | RS27695 |
| NO: 189) | |
| GGGTGCCGAAGACCTGAAGAAGTAACGAACCTGCGCTTCGCGCATCGCGGGCAAGCCCGCTCCCACATTGGATCG | PS417_ |
| CGGTCCCCTGTGGGCGCCGGGCTTGCCCGCGATGAGGCCCTCACAGCCGACTAAGATCCATAACC (SEQ ID | RS07595 |
| NO: 190) | |
| GGGTGGCAAAGGTTTAAATTAATTTTCAGCAAATTTGCACCGGGTTGGCGCGGGCAATTTCCTTGAACTATCCAGG | PS417_ |
| GCCGCAGGCTTCTGCTGCATAGGCGCTCGCTATTTTTACCCGCGCCATTGATTGGAGAACCACC (SEQ ID NO: 191) | RS12530 |
| GGGTTGGCGGCAAAACATAAAGCCAAACCTGCGGCCGGCGATGAAATGCCTGGGCAAAAATAATTGAAACTCTCC | PS417_ |
| CAAACGGCCTTGGGTCAATCTTTAGGTAATGACCAAAACCCTTGATTGTTCTCAGGAGAAATACC (SEQ ID NO: 192) | RS06330 |
| GGTCGAGTGATCGGAACGCTTTCACTGAAGCGCTTTACAGCAAAGTCGGAAGATTGGCACGGGAGTAGACTCCTGC | PS417_ |
| TGTCACACCTGATGCACGCTAAAAAAATACTAGATAGGACCGAGGCCAACCCGTGAACCCAGAC (SEQ ID NO: 193) | RS06610 |
| GTTCACCCTTGAAGAAGCCCCGAAACGTTTCAGCGTTTTCGGGGTTTTTTTGTTTTCGTTCATCGGGAACCAGACACC | PS417_ |
| TCTTTTCCCTTCTAAGCTTCTGGCAGATACCCACGCTGCTTCGTCCCTCCAGGAGTTTTTCC (SEQ ID NO: 194) | RS26035 |
| TCAGGGAAAGCCTCAGGAATGATGCGCAGTTTTGTGACTGACAACCCTTCGTCCAGGTCACGCCGCACGCAAATTA | PS417_ |
| GCTTGAATCTTTGGGACGCGCAGCCTGTCCACACACTATAAGCCCCACAGTATGAGGATTGGCC (SEQ ID NO: 195) | RS24450 |
| TGCCCGACCGCCCAGATTCAGCGCAGGCCTCAGGGCCTGCGCACCAAGGCAAAGCAAAAGCTGTACCACACAATTG | PS417_ |
| CACTGAACCTTCACCGAAGCCGGTGACTCGAAAACGTACAGATCAGCCAATCACGGAGATACCC (SEQ ID NO: 196) | RS19275 |

TABLE 6
Primers used.

| | |
|---|---|
| F-03A09-map- | CTCTTTCCCTACACGACGCTCTTCCGATCTTTACTGGATCTATCAACAGGAGTCC (SEQ ID |
| internal | NO: 197) |
| F-03A09-barcode- | CTCTTTCCCTACACGACGCTCTTCCGATCTCTGTGCCAAGCGAACTGAGTGTATTTGAGC (SEQ ID |
| internal | NO: 198) |
| R-03A09-map- | GAGTTCAGACGTGTGCTCTTCCGATCTGGTACCTTTCTCCTCTTTAATG (SEQ ID NO: 199) |
| internal | |
| R-03A09-barcode- | GAGTTCAGACGTGTGCTCTTCCGATCGGCAGTTTACCAGTAGTACAGATGAACT |
| internal | (SEQ ID NO: 200) |
| Barcode_R1 | GGCAGTTTACCAGTAGTACAGATGAACT (SEQ ID NO: 201) |

### Method

Bacterial strain. We based our study on Pseudomonas simiae (WCS417r) that was originally obtained from Dr. Come Pieterse (Utrecht University), as described in Cole et al 2017. This strain was domesticated by a landing pad integration, resulting in SB599 as described in Wang et al 2020.

Barcoded promoter library design. We used an annotated genome of Pseudomonas simiae (WCS417r) for a library design (NCBI: NZ_CP007637). We first categorized the genes into three subgroups based on their length of intergenic regions to the upstream genes. We refer to them as group I-III and each group contains genes whose intergenic distances are 1-30 bp, 30-140 bp, and longer than 140 bp, respectively. We then extracted 140 bp regions immediately upstream of the start codon for all genes. These processes were done by using Geneious software. We then added 30 bp priming sites for both ends of the 140 bp sequences for the cloning purpose, and ordered the three groups of 200 bp DNA library to Twist Bioscience. These libraries are referred to as promoter libraries for convenience.

For the DNA barcodes, we designed 23 bp sequences with initial three nucleotides set to be CGT, and the next three nucleotides to be GGA/AGG/GAG, and the rest of 17 bp to be randomized. The first three letters were set to distinguish different strains in case needed for future studies. The second three letters are associated with group I-III libraries to distinguish them respectively. We then added 30 bp priming sites for both ends of the sequences for the cloning, and ordered the three groups of 83 bp DNA library to Integrated DNA Technologies (IDT). These libraries are referred to as barcode libraries for convenience.

Plasmids for library transformation. pW26 was modified to contain BsaI sites, sfGFP, Apr, lox2272, lox5171.

Library synthesis, assembly, transformation, and conjugation. The procedures described in this section were conducted in parallel to produce group I-III libraries respectively. The oligo pools of promoter libraries and barcode libraries were resuspended to 10 ng/μl and 10 μM, respectively. dsDNA pools were generated by running PCR reactions, using 1 μl of promoter libraries, 2.5 μl of barcodes libraries, and 2.5 μl of 10 μM 5′-forward primers. The reactions were performed for 7 cycles in 50 μl total volume using Q5 polymerase (NEB: M0493L). The amplified products were subsequently gel-purified.

For the assembly, the vector (modified pW26) was first digested by BsaI enzyme (NEB: R3733S) overnight and dephosphorylated by a quick dephosphorylation kit (NEB: M0525S) the next morning. The digested vector was gel-purified. Subsequently, Gibson assembly reactions were performed with ˜23 ng of digested vector and ˜12 ng of dsDNA pools in 10 μl total volume using a master mix (NEB: E2621S).

The assembled plasmids were then transformed into E. coli pir+ competent cells (Lucigen: ECP09500) in order to amplify. The plasmids were first diluted by 5 fold in water and 1 μl were gently mixed with 20 μl of competent cells on ice. After the electroporation, a small aliquot of the recovery cultures were diluted and plated to determine the cloning efficiency, while 500 μl of the cultures were diluted in 3 ml LB and spread onto bioassay dishes (VWR: 73520-774) with LB agar and 50 μg/ml apramycin. The coverages of all group I-III libraries were determined as >200× by dividing the number of colony-forming units by the size of the designed library. The plasmid DNA was then extracted from the colonies on dishes with a midiprep kit (Promega: A2492). The extracted plasmids were subsequently transformed into E. coli WM3046 competent cells (Lucigen) to be used for conjugation as a donor strain. The cells were recovered in LB+0.3 mM diaminopimelic acid (DAP) for 1.5 hr at 30 C, and spread onto bioassay dishes with LB agar, 50 μg/ml apramycin and 0.3 mM DAP. In the next day, the cells were scraped from the dishes and resuspended in 10 ml LB medium.

The library integration into the chromosome of Pseudomonas simiae (WCS417r) was implemented by CRAGE via conjugation as described in (Wang 2019, Wang 2020). We used P. simiae strain (SB599) as a recipient strain that harbors Cre recombinase gene and kanamycin-resistant marker (Km^{R}) flanked by two mutually exclusive lox sites (lox2272, lox5171) in the landing pad. For recombination, the recipient P. simiae strain was first grown in LB medium at 28° C. overnight. The Donor WM3046 cells and recipient cells were washed twice with LB medium respectively, then mixed at 4 (donor):1 (recipient) ratio in OD600 to be 1000 μl in the final volume. The mixed cultures were washed once again, resuspended in 100 μl of LB+0.3 mM DAP medium, placed onto LB agar plates containing DAP, and incubated overnight at 28 C. In the next day, the entire colonies were taken by using a loop and resuspended in 1 ml LB. The cultures were washed once and spread onto bioassay dishes that contained LB agar and 50 μg/ml apramycin. In the following day, the colonies were scraped from the dishes, resuspended in 10 ml LB with 10% glycerol and stored at −80 C for later experiments.

In vitro cell culturing and nucleic acid extraction. 10 μl glycerol stock containing each group I-III library was inoculated in 3 mL fresh LB supplied with 100 μg/ml apramycin and grown until saturation at 30° C. in a shaking incubator at 200 rpm. 3 or 30 μl of the cell cultures were transferred into 3 mL fresh growth media supplied with different nutrients and grown overnight. In the next day, the cell cultures in the mid log phase were diluted to OD₆₀₀=0.05-0.1 in pre-warmed growth media and cultured again at 30° C. (or 37° C. for heat stress condition). When OD₆₀₀ reached close to 1.0 (˜8×10⁸ cells/ml), 2 ml of the cultures were pelleted and the supernatant was removed. The pellets were then resuspended by 750 μl DNA/RNA Shield and the cells were lysed for 10 minutes with 0.1 & 0.5 mm beads at the maximum speed. Here we performed the parallel DNA and RNA extraction by following ZymoBIOMICS DNA/RNA Miniprep protocol (Zymo Research: R2002).

The growth media were based on the M9-buffered minimal medium. The base medium contains 1× M9 minimal salts (Gibco: A1374401), 2 mM magnesium sulfate, 0.1 mM calcium chloride, and 10 μM ferrous sulfate. Additionally, one of the following was supplied as the primary carbon source: 20 mM glucose, 40 mM glycerol, 20 mM citrate. For salt stress conditions, sodium chloride was additionally supplied to be 300 mM in the final concentration. For synthetic root exudate medium, two stock solutions were prepared and filter-sterilized. One of them, 2.5-fold K0, solution contains 50 mM fructose, 50 mM glucose, 50 mM sucrose, 25 mM succinic acid, 25 mM malic acid, 12.5 mM arginine, 12.5 mM serine, and 12.5 mM cysteine, which represent a range of compounds commonly reported occurring in root exudates (Griffiths et al., 1998). The other one, 5-fold K1 solution, contains 12.5 mM (NH₄)₂SO₄, 2.5 mM NH₄Cl, 12.5 mM Ca(NO₃) 4H₂O, 5.0 mM NaH₂PO₄ H₂O, 2.5 mM KNO₃, 3.75 mM MgSO₄ 7H₂O, which represent common NH₄—NO₃ nutrition composition (Kraffczyk et al., 1984). These two stock solutions were proportionally combined and diluted with H₂O to make the final one-fold concentration. 0.5% Trace Mineral Supplement (ATCC MD-TMS) was then added. pH was adjusted to 7.0 by NaOH and filter-sterilized to prepare the final medium.

Plant growth conditions. A. thaliana Col-0 seeds were surface-sterilized in 70% ethanol for 5 minutes, followed by 50% bleach plus 0.1% Triton-X100 for an additional 5 minutes. Sterilized seeds were washed 5 times in sterile water, and stratified in the dark for 2 to 4 days at 4° C. After stratification, 100 seeds were plated on a nylon mesh filter (100 micron pore size, cut to an area of approximately 8 cm²) placed on top of phytagel plant growth media (0.5×Murashige and Skoog basal salts [PhytoTech Labs: M404], 2.5 mM MES [Sigma-Aldrich: M3671], 0.6% phytagel [Sigma-Aldrich: P8169], pH adjusted to 5.7) in a 10 cm square petri dish. Seedlings were grown upright in a Percival incubator (Geneva Scientific: CU36L5) for 10 days prior to contact with bacterial cells. The incubator was set to a long day mode (16 hrs lightning, 8 hrs dark) at 22° C.

Root colonization assay. 10 μl glycerol stock containing each group I-III library was inoculated in 3 mL fresh LB supplied with 100 μg/ml apramycin and grown for approximately 5-6 hours at 28° C. in a shaking incubator at 200 rpm until the culture reached the late exponential phase. 30 μl of cell culture was then transferred into 3 mL fresh liquid-type plant growth media (no phytagel added) supplied with 40 mM glycerol as a carbon source. After ˜20 hrs incubation in the shaker, cells were harvested by centrifugation (3,000 g for 1 minute) and washed 3 times by resuspending in liquid-type plant growth media (no glycerol added) and pelleting the cells. After the washing, the cells were resuspended in liquid-type plant growth media and were normalized to OD 0.5. 50 μl was then spread onto fresh phytagel plates with plant growth media using 5-10 sterile glass beads. 10-day-old Arabidopsis seedlings grown on a sterile nylon mesh filter were transferred onto the phytagel plates inoculated with cells. The plates were then grown upright in a Percival incubator at 22° C. with 16 hrs lightning and 8 hrs dark setting. After 3 days of initial colonization period, the root samples were collected every 24 hours over a 2 days period.

Nucleic acid extraction. We used ZymoBIOMICS DNA/RNA Miniprep Kit (Zymo Research: R2002) with a modified protocol to extract DNA and RNA. This modification ensures an efficient isolation and lysis of bacterial cells by using two different sizes of bashing beads. For the sampling procedure, seedlings on plates were cut below the root/shoot junction, and the isolated roots were placed into 2 ml tubes. Roots from one plate (30-50 seedlings, ˜10 mg) were pooled into a single sample. The pooled roots were then vortexed for 5 seconds in 800 μl liquid-type plant growth media to wash out loosely adhered cells from the surface of the roots. After the buffer was removed, 800 μl DNA/RNA Shield and 2 mm beads (Zymo Research: S6003-50) were added into the tubes. The tubes were then placed onto an adapter (Zymo Research: S5001-7) attached to a vortex Genie2. The samples were ground for 10 minutes at the maximum speed. After breaking apart the root tissues in the first step, the lysed samples were transferred to the tubes containing 0.1 & 0.5 mm beads provided in the R2002 kit. The samples were ground for another 40 minutes at the maximum speed to lyse bacterial cells. We then followed the parallel DNA and RNA extraction procedure with DNAse treatment described in the manufacturer's protocol. DNA and RNA were eluted in 70 μl of ddwater and stored at −20° C.

Colony-formation-unit measurement. Seedling samples were collected and washed once with similar procedures as nucleic acid purifications. After the washing buffer was removed, fresh 800 μl liquid-type plant growth media and 2 mm beads (Zymo Research: S6003-50) were added to the tubes. The tubes were then placed onto an adapter attached to a vortex, and the roots were ground for 10 minutes at the maximum speed. The lysed samples were diluted for 10,000 fold by liquid-type plant growth media and 100 μl were spread onto LB plates. Plates were incubated at room temperature for two days and colony numbers were enumerated. As we obtained about 2×10⁶ cells/mg-root and used approximately 10 mg of roots for each experiment, the number of collected cells was about 2×10⁷. This number is about 100 times lower than the one in the liquid media experiment, as we typically collected 1.6×10⁹ cells where we sampled 2 ml of cell culture (8×10⁸ cells/ml/OD₆₀₀).

Targeted sequencing library preparation from genomic DNA. To create targeted sequencing libraries, we amplified either genomic DNA or cDNA in a two-step PCR process. For genomic DNA samples, purified DNA was amplified by PCR using two primers flanking the barcode region. All primers used in this study are listed in Table6. We ran 6 tubes of 50 μl reactions with each reaction tube containing the following components: (1) 10 μl of Q5 reaction buffer (New England Biolabs: B9027S); (2) 1 μl of dNTP (New England Biolabs: N447L); (3) 2.5 μl of 10 μM forward primer; (4) 2.5 μl of 10 μM reverse primer; (5) 10 μl of purified DNA; (6) 0.5 μl of Q5 polymerase (New England Biolabs: M0493L); (7) 10 μl of betaine solution (Sigma-Aldrich: B-0300); and (8) 13.5 μl of ddwater.

PCR was performed by the following settings: 1) initial heating for 30 seconds at 98° C., 2) 14 cycles of 10 seconds at 98° C. and 60 seconds at 72° C., 3) followed by a final extension at 72° C. for 60 seconds. Following PCR, 6 reaction tubes were pooled into a single sample. The samples were then purified using the DNA Clean & Concentrate Kit (Zymo Research: D4013) and eluted to 20 μl ddwater. DNA was quantified by a Qubit fluorometer (Thermo Scientific). For the second-step PCR, indexes and Illumina P5 and P7 adaptors were added to the samples. 1 tube of 50 μl reaction was prepared with each reaction containing the following components: (1) 10 μl of Q5 reaction buffer (New England Biolabs: B9027S); (2) 1 μl of dNTP (New England Biolabs: N447L); (3) 2.5 μl of 10 μM indexed P5 adapter primer; (4) 2.5 μl of 10 μM indexed P7 adapter primer; (5) ˜30 ng of diluted DNA samples; (6) 0.5 μl of Q5 polymerase (New England Biolabs: M0493L); and (7) ddwater adjusted to total 50 μl.

PCR was performed by the following settings: 1) initial heating for 30 seconds at 98° C., 2) 5 cycles of 10 seconds at 98° C., 15 seconds at 60° C. and 30 seconds at 72° C., 3) followed by a final extension at 72° C. for 60 seconds. Following PCR, the samples were run for gel-purification and eluted in 30 μl ddwater by a cleanup kit (Macherey-Nagel: 740609.050). The quality of the amplicon samples was validated by Bioanalyzer DNA Analysis (Agilent: 5067-1504).

For RNA samples, 70 μl of purified RNA was first treated by DNAse (Thermo Scientific: AM1907) to remove trace genomic DNA. The quality of RNA samples was checked by Bioanalyzer RNA Analysis (Agilent: 5067-1511). The samples were then concentrated by using the RNA Clean & Concentrate Kit (Zymo Research: D1013) and eluted to 15 μl ddwater. 10 μl of the concentrated samples was used for cDNA synthesis. Selective reverse transcription was carried out by SuperScript II Reverse Transcriptase (invitrogen: 18064022) and a Barcode_R1, following the manufacturer's protocol. The synthesized cDNA was amplified by PCR using a similar protocol to the genomic DNA samples. We prepared 8 tubes of 50 μl reactions with each reaction tube containing the same components as described above, except 2 μl of cDNA and 21.5 μl of ddwater. PCR was performed by the following settings: 1) initial heating for 30 seconds at 98° C., 2) 22 cycles of 10 seconds at 98° C. and 60 seconds at 72° C., 3) followed by a final extension at 72° C. for 60 seconds. Following PCR, 8 reaction tubes were pooled into a single sample. The samples were then purified using the DNA Clean & Concentrate Kit (Zymo Research: D4013) and eluted to 20 μl ddwater. The quality of PCR products was validated by Bioanalyzer High-sensitivity DNA Analysis (Agilent: 5067-4626). For sequencing purposes, the second-step PCR was carried out as described above in order to add indexes and Illumina P5 and P7 adaptors.

For the samples collected from liquid culture, we reduced the cycle numbers of the first PCR to 12 cycles for genomic DNA samples and 20 cycles for cDNA samples. Otherwise, the same procedures are conducted. The amplicon samples were sequenced on Illumina NovaSeq platforms with 150 bp paired-end sequencing, targeting an average of 10-20 million reads per sample.

Sequencing library preparation to associate barcodes with promoters. 10 μl glycerol stock containing each group I-III library was inoculated in 3 mL fresh LB supplied with 100 μg/ml apramycin and grown overnight at 30° C. in a shaking incubator at 200 rpm. 300 μl of the respective saturated cultures were pelleted and the supernatant was removed. The pellets were then resuspended by 750 μl DNA/RNA Shield and the cells were lysed for 10 minutes with 0.1 & 0.5 mm beads at the maximum speed. We then extracted DNA for each group I-III sample by a miniprep kit (Zymo Research: R2002). For the first PCR, a region covering from promoter to barcode was amplified by specific primers (Table 6). We used the same PCR setting as the barcode amplifications described above, except the cycle number set to 15. Subsequently, the second PCR to add indexes and Illumina P5 and P7 adaptors was carried out in the same procedure as described above. The amplicon samples were sequenced on Illumina NovaSeq platforms with 250 bp paired-end sequencing, targeting an average of 100 million reads per sample

RNA sequencing library preparation. For the samples collected from liquid culture, ribosomal RNA was removed from 1 g of total RNA with rRNA depletion kit (New England Biolabs: E7860S). The treated RNA was eluted in 6.5 μl of ddwater. Sequencing library was then created by TruSeq Stranded mRNA Library Prep kit (Illumina: 20020594). We started the procedure by adding 3 μl of rRNA-depleted RNA samples into 15 μl FPF, then followed the manufacturer's protocol. The quality of the libraries was validated by Bioanalyzer DNA Analysis (Agilent: 5067-1504). Samples were sequenced on Illumina NovaSeq platforms with 150 bp paired-end sequencing, targeting an average of 5 million reads per sample.

For the root samples collected from Arabidopsis seedlings, we didn't perform the rRNA depletion, since our aim was to obtain the fraction values of mapped reads to Arabidopsis and P. simiae. We used 60 ng of total RNA and started the procedure by adding into 18 μl FPF. We then proceeded the same steps as mentioned above. Samples were sequenced on Illumina NovaSeq platforms with 150 bp paired-end sequencing, targeting an average of 400 million reads per sample.

Computational process to map barcodes to promoters. A custom shell script with BBtools and R script with Biostrings and dplyR packages were used to map barcode sequences to their attached promoter sequences. Briefly, the regions of 140 bp promoter and 20 bp barcode (excluding initial CGT set to P. simiae WCS417) were first extracted from read 1 sequences. The pairs of each promoter and barcode set were selected if they were detected more than 100 times from the sequencing reads. The barcodes mapped to two or more promoter sequences were discarded from the further analysis. Then, the promoter sequences that have exact matches with native sequences were selected. These analyses were carried out for each group I-III sequencing data. We found 92% of total promoters (5090 genes) were assigned with unique barcodes and each promoter was on average covered by 50 unique barcodes.

Quantification of promoter activities. Barcode sequences were extracted and counted from the sequencing files by using a custom shell script with BBtools and R script with Biostrings and dplyR packages. In this pipeline, barcode counts were first normalized to sequencing depth per each sample and summed for each promoter. To calculate individual promoter activity (A.U.), we divided the barcode counts derived from mRNA-derived cDNA by the barcode counts from genomic DNA. To show library coverage in figures, we normalized the obtained DNA barcode counts to per million (CPM).

Statistical analysis was carried out by using edgeR and limma packages with custom modifications from Law et al. Briefly, the barcode counts for each promoter were transformed to log 2 values using edgeR's cpm function. We then performed limma-trend analysis to identify differentially expressed promoters, and obtained the log 2 fold change and p-values for each promoter. The p-values were further corrected for multiple hypothesis testing by Benjamini & Hochberg correction.

Functional analysis. Operon structures were predicted by using Rockhopper. Genome sequence of P. simiae WCS417 and RNA sequencing reads obtained from root colonization assay and growth in 40 mM glycerol were feeded as input files. Before the analysis, RNA reads mapped to rRNA and Arabidopsis genome were filtered out from the input data. As a result, 1153 operons were identified and 2361 genes were predicted to be a part of the operons. KEGG pathway analysis was performed by using kegga function included in the limma package in R. If the identified promoter drives operon, a cluster of genes downstream of the promoter was feeded as input. KEGG IDs were obtained from the IGB website.

RNA sequencing data analysis. RNA sequences data were processed by using a custom shell script with BBtools and R script with dplyR, edgeR and limma packages. In this pipeline, we first filtered out reads mapped to 5s rRNA, 16s rRNA, 23s rRNA, and Arabidopsis genes when contained, using BBsplit function. Then, reads were mapped to P. simiae WCS417 genome by HISAT2. The mapping results were used to calculate read counts over each gene by FeatureCounts. The count data were converted to log 2 CPM values using edgeR's cpm function. The log 2 fold change values between conditions were then obtained from limma-trend analysis.

Conducted an Infection assay to identify promoters responding to plant infection. The method included: (1) Streak P. syringe from four edges onto KB+Rif plate. Incubate at room temperature. (2) Collect all colonies and resuspend in 1.5 ml, 0.04% Sliwet in 10 mM MgCl2 solution (4 μl Sliwet in 10 ml). (3) Vortex 10s. (4) Centrifuge at 4000 rpm for 1 min, then resuspend by 1 ml solution, pipetting to mix. (5) Set OD to 1.0. (6) No bacteria control to be prepared (0.04% Sliwet only). (7) Treat leaves using sticks until all the plant leaves look completely wet to maximize the impact of infection. Make sure one-by-one moving a stick from left to right. Be careful to apply uniformly if there is a region where plants are dense. (8) Wrap by micropore tape. (9) Transferred plates into an incubator and collected nucleic acid samples for two days. Below is what plants looked like in day2. Control plants have more leaf and root growth than plants treated with a plant pathogen, such as Pseudomonas syringae.

TABLE 7
Promoters that are down-regulated on day2 of P. syringe infection.

| | locus_ |
|---|---|
| | tag. |
| Down-regulated promoters | refseq.x |
| AAAAATCCGCGAACTGTTCAGACCAGTCCGACCATCGCAACGCAATAAATCCCTGGCTTGCAGGCTTTATTGAGAG | PS417_ |
| TGATGGCATGGGCTGTGCACTGAGCAAATCCAGAAAATTCCTTCGCCCTTCTCGGAGAGTTGCC (SEQ ID NO: 202) | RS01155 |
| AAAGGTAAATGACCGGTAGATATCTAATTAAAGGAAAAGCTGGTTGGAAATTGACCTAGAGGCTTGCTTTCTCTAG | PS417_ |
| AATCGCCGGTCTCTTAAAACGGGGGCCATTCCGGCCCGTTGTGGACGAACCAGGTAACAACGCC (SEQ ID | RS00005 |
| NO: 203) | |
| AGGTGATCGACGTGGACGATTTCAGCAAGGAAGAGCTGACCCAGGCTGAGGGTAAAGTCCGCTGATCCCAGTGG | PS417_ |
| TCATATAAATACGGGCGCGAGAGCGATATACTCTCGCGCCCGTTTTTGCTTTAGAGGACTTATCTC (SEQ ID | RS18735 |
| NO: 204) | |
| CAAGCTCAACGGCGAACGCCGCTTTGAGTTCGCCCAACTGCAGACCAACAGCGTGGAAGAAGCCAAGCAAGCCCT | |
| GGAGAAAATTCACGATGCCAGTGATGAAATCACCGACATCAATGTGAGCAAGGCGCTGTAAGCCA (SEQ ID | |
| NO: 205) | |
| CCGCCTTCGCGAGCAAGCCCGCTCCCACATTTGTTTTGTGATGACCACACAAATTGCTGTACATCCATACAGATAAA | PS417_ |
| GGTTGATCAAAGCCCTCCGACTGAGCATCCTGCAAGGCATCAACCCGATCTGATGTGATGACC (SEQ ID NO: 206) | RS17445 |
| TCGCAGCCTCGCGAACGCTCGACAGCTCCCACCCGACAGCAACTGTACGGATAAATCCGCACGCAGCTGTAGACCC | PS417_ |
| TTGATCGGTGAGGGATGTGGCTAGATGGGCTTCCCTTGAAGCCCTCTAGATTCGGTTCTTCTCC (SEQ ID NO: 207) | RS20980 |
| TGCCGGTACCACCCTGGCCAACATTGCCGCCTGGGCCGATGGGCGGGCGCAGAACCTTGTCGAAGGATGATCAGC | |
| GGTCACATACCGGGCTCATGATGAGCCAGCACCGTGATAGGATGCCGCGCCTATTTGGAGGATCC (SEQ ID | |
| NO: 208) | |
| TGCGATACATTGCTAATTGCACTAAATTTACATTCTTTACACTTTTTCCTTACGGTTTTTACGGACTCGTTCGTCCCGG | PS417_ |
| CGTTTTACAGGGCGGATAAGAATCAATACCATTGGCGCCTTTCCGAATTCAGGTATTGCCC (SEQ ID NO: 209) | RS23790 |
| TGCGCCGTCCAGTGATCCCTACCAACGGATTATCGCCTGAACGGTAACAAAGATTCACAGGAATGCCAGCTTATCG | PS417_ |
| TCCTCCCTATCGCCCCCTAGAATTGCGCCACCTACCCCCGCATCACCTCAGGAAGTCGCTTGCC (SEQ ID NO: 210) | RS23050 |

TABLE 8
Promoters that are up-regulated on day2 of P. syringe infection.

| | locus_ |
|---|---|
| | tag. |
| Up-regulated promoters | refseq.x |
| AACATCGTTTGAATCGCTAAACACTCTAAAGAGCTGTCAACACTCACAGGTTACTGAACCGACGCACCCTGCTAGC | PS417_ |
| CTGAGCCAGTCGAACTCAATCCTCGTCTGGCTCACGCCTTTACCATGTGCGAAAGGAATCGTCC (SEQ ID NO: 211) | RS12670 |
| AATGACCGCTGTCCCTGCTGGAAATCGATACCTGCCACACCGCAGATCCAATGTGGGAGCAAGCCCACTCCCACAG | PS417_ |
| TTGGCCGGCGGTGTGGCCGGCTATGAGTTCGAGAGGAAATGGCCGCACAAATGGAGAGGACTGT (SEQ ID | RS03665 |
| NO: 212) | |
| ACAATAAGCACTTTGTATTGTGTACAAAAAACCCTAAAGATTTACCGGCTACGGGCCGAAACAGCAGTGATAGCAA | PS417_ |
| ATCGCCGCAAACCTGATACAGGCGCGCGACGATTTGGCCGCCCTTCACGCTGATGGTCGTCTCG (SEQ ID | RS13395 |
| NO: 213) | |
| ACATTTGCCCCTCAGCCTGAAGACCGGGTCCCACTTTTCTGCTCATAAGCTAATTCGAAAACGTTATTTATTTTCGGA | PS417_ |
| TTCTTAGATCATTTAGTCTCCTCGCATGCCGACCCTCGGCCGCCTGATTGAGGAGCTTCCCC (SEQ ID NO: 214) | RS26580 |
| ACCCCCCGCAAAACACCCTGACCACGCAACTCAATATCCGCTGATCAATCAGCTTGAACGCTCAATACCCCGGGAA | PS417_ |
| GTCGTACCCCTGAGACGGCGATTTTCCTGAACGCCGCGGATGTAGACCAACCCGTGAGGTGTTT (SEQ ID | RS04050 |
| NO: 215) | |
| AGGGCTGGGCGCAAAGCGCGCAATTAAACCATAACCGAAGGCATGACAATTTATCGCTGGCGGTAGGTAGACAC | PS417_ |
| CGTAGTTTGTGGTTAACTTCGACCTCTCGAATTCGTTAAACCCACATCAGCATAAGGATTCCGTTC (SEQ ID | RS15310 |
| NO: 216) | |
| AGTTAGGGGTTTATTTCAAAGATGAACTTCGGCTATTTCCCATGGCCCCTGTTATTCCTGAGGGATATTTACCAAGC | PS417_ |
| AATTGTCAAGTATTTCCTGTACGATGCCTGGCCAAGTTAATTTTCTACAGGTTAGTGGCCGCC (SEQ ID NO: 217) | RS08110 |
| ATGCCGGGATCGTCGTGAAGGGCTGCGACGCGATGGAATAAAGCGATGCCAGTTTGATGTCCGCTGGGCGGGAT | PS417_ |
| CGAACTACTTATAGTTTGGTACGACGGTACCAATATCTTCTGCCTGACAGATTTAGAGGAACGACC (SEQ ID | RS07790 |
| NO: 218) | |
| CACCACTGCCGCCAGGGCAGAGAGCAATTTATAGTCGAACATCAGTTTCTCTAATGAGCGATCAGCAATATTTGTT | PS417_ |
| TTTCTTATACAGCCTGTGTCAGGAGAATAGCCAGCATCGCTTCTCTCTTTATAAGGAACCTCAC (SEQ ID NO: 219) | RS06215 |
| CCAGCAAGCTGGCTCGTACTCTGGCCGCAGTTCGCGAGCAAAAAGAAGCTGCCGCAGCCTAAGGCTGAGCGACTT | |
| CTCTCGCGTATTTTTGTTTATTTCGATGGCCGAGTAGGCCGTCCCCCAATTCAGGAAATACAGCA (SEQ ID NO: 220) | |
| CCATCGCCTTACGGCGAGAATGAGTTTTTCCGTAAGGCCGTGCGTCGTCACGAGCTGCTTTGGGTGCTGAAGCAG | |
| GACTGAAATTGACTTTTTTCGTCGGGCTGCGTGCGCGTAGCAGCCCGATTGCCTCATGGAGTGAT (SEQ ID | |
| NO: 221) | |
| CTGTGAATCTGTAAAGTTCAAGCTTTCTGCAAAAAAACGCAAATAGAGCTGAATTATTTCATGTCCGCTTGTATCTG | PS417_ |
| AACTGACAGAGCGGTGCCATCGCAACGATGGCACCTCCCCTGTTCTGCTTTAGAGTGCTCCGC (SEQ ID NO: 222) | RS13355 |
| GATCCACTGCGGCAACTGGCGATGATCCGTGCCACGGCCGAGCTGTGCGTTCGACCTACTTGATTTGAGGAACCGT | |
| CCTGCATCTTCACCTGGGCCGACAGCCATAGCCTGAGCGTTCACTGACCGAACAGGATGGAACC (SEQ ID | |
| NO: 223) | |
| GGCAAGGCTATCCTTAATGCCTTGCCGCCGTTCAGGCGCGAAATTTCTTAAGCCACAGTGGGCGGATTCGCCCACT | PS417_ |
| TCGTGGTCTATCTGACTGTCATTCATCAGGCCATTCATCGGCCGCCTGGGAGAACCTTGCTCGT (SEQ ID NO: 224) | RS21315 |
| GGCATCCGCCCTGTTTGGTTCCACCACGGGCCTTTCCACAACGGCGACTGCATGCGCACGAAAAAACCACTCAACT | PS417_ |
| GTATTTTGACGCTCGCTTGACCCACTGTTAGTGTCCGGCTCCATCCCTACACGGAGTGCTTTCG (SEQ ID NO: 225) | RS24350 |
| GGGTGGCAAAGGTTTAAATTAATTTTCAGCAAATTTGCACCGGGTTGGCGCGGGCAATTTCCTTGAACTATCCAGG | PS417_ |
| GCCGCAGGCTTCTGCTGCATAGGCGCTCGCTATTTTTACCCGCGCCATTGATTGGAGAACCACC (SEQ ID NO: 226) | RS12540 |
| GTGCCGCAGATCTGGATCGGCGCCAAGCATATCGGTGGTTGCGATGACCTGTTCGCCCTGGAGCGCGCGGGTAAA | |
| CTCGATGCGCTGCTGCTCGTCTGATGCCTAAACCCTAAAAGACCCCAAGATCACAAGGATCTGCG (SEQ ID | |
| NO: 227) | |
| TGACCTGGTCCAAACCTGCTTACACTGATCTGCGCATCGGTTTCGAAGTCACCATGTACTTCGCCAGCCGTTAATCC | PS417_ |
| GCTGGGTAATACAACGCCTCGGTTCGCCCGGGGCGTTTTTGTTTTGAGCTTGATGGAGCTGCC (SEQ ID NO: 228) | RS02595 |

This example describes the following: (1) A high-throughput assay to characterize transcriptional activities of root colonizing bacteria. (2) RB-piseq (random barcoded-promoter insertion sequencing) is described in U.S. Provisional Patent Application Ser. No. 63/402,816. The method comprises (a) extracting of 5′-regulatory regions (about 140 bp region) from a genome, such as a bacterial genome, such as P. simiae genome; (b) constructing a barcoded promoter library, wherein each member of the library comprises a construct from 5′ to 3′: 5′-regulatory region barcode reporter gene (such as sfGFP); (c) inserting each library construct into a chromosome of a host cell using CRAGE; and (d) optionally testing the library of constructed host cells for one or more phenotypes. (3) cDNA is constructed from which the barcode is amplified. It is enriched and then sequenced. The process is repeated similarly for barcodes on genomic DNA. The value of RNA barcode/DNA barcode is calculated to produce the promoter activity. (4) Characterization of promoter activities during root colonization (such as using a P. simiae library). (5) KEGG pathway analysis to assay change of metabolic activity and biofilm formation. (6) A competition assay to test the pathogenicity of mutant plant phenotypes.

This example describes the following: (1) Development of RB-Piseq to characterize in planta promoter activities. (2) Identification of about 200 regulated promoters during colonization. Reduced metabolism results in biofilm formation. (3) Identification of several genes that affect colonization efficiency. (4) RB-Piseq is useful for the characterization of microbes and the discovery of new functions of genes.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

All cited references are hereby each specifically incorporated by reference in their entireties.

What is claimed is:

1. A method to quantifying transcriptional activity of promoters, the method comprising: (a) extracting a library of 5′-regulatory regions from a genome; (b) constructing a barcoded promoter library from the library of 5′-regulatory regions, wherein each member of the barcoded promoter library comprises a construct comprising from 5′ to 3′: 5′-regulatory region-barcode-reporter gene; (c) inserting each member of the barcoded promoter library into a chromosome of a host cell using chassis-independent recombinase-assisted genome engineering (CRAGE) to produce a library of host cell containing the construct; (d) testing the library of host cells to a test condition; (e) identifying the promoters that are up-regulated or down-regulated in response to the test condition; and (f) optionally identifying each gene corresponding to each identified up-regulated or down-regulated promoter in the host cell or in nature.

2. The method of claim 1, wherein the test condition is a change in the environment.

3. The method of claim 2, wherein the change in the environment is introduction to the proximity of a host organism or infection of a host organism.

4. The method of claim 3, wherein the host organism is a plant.

5. The method of claim 3, wherein introduction to the proximity of the host organism is the introduction of the host cell to a root region of a plant.

6. The method of claim 1, wherein the 5′-regulatory region is about 140 bp in length.

7. The method of claim 1, wherein the reporter gene is green fluorescent protein (GFP).


SEQUENCE LISTING (228 sequences)

SEQ ID NO: 1 - DNA, 140 bp, Pseudomonas simiae
  source 1..140: mol_type = genomic DNA
aataagtcgagcggggcggcagtggcggtcatcgcaggcttcttcccaggcagctataaaagaagggcgaagtatacccgagcgtcctggctggcggatcgctataatcggcggctttgtttatgagtgcggagtaaggg

SEQ ID NO: 2 - DNA, 140 bp, Pseudomonas simiae
  source 1..140: mol_type = genomic DNA
aatttgcaagattcgtcaaaaaccctggattttggtcggattgtcctacaaaaacgctctatctcctgcattacagcctatagcgcgatcttaattttattgttactttccgcagcggtgatcaccacggagttctaata

SEQ ID NO: 3 - DNA, 140 bp, Pseudomonas simiae
  source 1..140: mol_type = genomic DNA
accgccgcgtgctggagaacaccccgcaaacttgatcagccgagcacttttcggcactctaccgagcttcgcggggacgaccccgcgacgcttccagcgcctaatccggggacgaccccacctaacgttaggaggtcatc

SEQ ID NO: 4 - DNA, 140 bp, Pseudomonas simiae
  source 1..140: mol_type = genomic DNA
acgccaaacatggaaagggcgcagaatctggcgtggaacgcattgatacaggtcagccattgcgttgcaatggcccctcgctttctacatacttgcggccgattttacctataaaaaaacctaaaccgtggaaccttaga

SEQ ID NO: 5 - DNA, 140 bp, Pseudomonas simiae
  source 1..140: mol_type = genomic DNA
acttccacggacgtcttggccggcaatttaggccgtggcggccacccaagcaagcgcttggctaaaacttgtgactggcgtttacaaaccaagcgcttgcttggtagtctcgcccacagccattcgaggaaggatttctc

SEQ ID NO: 6 - DNA, 140 bp, Pseudomonas simiae
  source 1..140: mol_type = genomic DNA
agcaaggacgccgcgccggttgctcaagcaacccgcagctgataggccgacagcgctaagacaaaccccggacaattccggggtttgtcgtttctgccagccccgctatcattcgctccattcttcgggggattcaactg

SEQ ID NO: 7 - DNA, 140 bp, Pseudomonas simiae
  source 1..140: mol_type = genomic DNA
aggcgttcgagggtcaggtcttcaaggggagggtgtttaaaggctcgcatgaaatgaatcataacaggggggtatcgtcgaataccatagttcgattaccatcgaactatcgaatcactcactcgctcctggagttctcc

SEQ ID NO: 8 - DNA, 140 bp, Pseudomonas simiae
  source 1..140: mol_type = genomic DNA
agggctgggcgcaaagcgcgcaattaaaccataaccgaaggcatgacaatttatcgctggcggtaggtagacaccgtagtttgtggttaacttcgacctctcgaattcgttaaacccacatcagcataaggattccgttc

SEQ ID NO: 9 - DNA, 140 bp, Pseudomonas simiae
  source 1..140: mol_type = genomic DNA
cacacctaacaagaaagcataagattagttcgccttctcatttgacaatcattatcattcagcataatttgttgcccgatgtgtaggaggcctcagcaaggacgccctcccctgacttcttttgcgacaaggtgatttcc

SEQ ID NO: 10 - DNA, 140 bp, Pseudomonas simiae
  source 1..140: mol_type = genomic DNA
caccactgccgccagggcagagagcaatttatagtcgaacatcagtttctctaatgagcgatcagcaatatttgtttttcttatacagcctgtgtcaggagaatagccagcatcgcttctctctttataaggaacctcac

SEQ ID NO: 11 - DNA, 140 bp, Pseudomonas simiae
  source 1..140: mol_type = genomic DNA
cagatcctacgacttggtcacatactttgcgctatcctcgctaatcgtgccgcctactgcatagaactggacagtcacaaaggctttgcggcatagtcgccgggttctgccgtacccacatcaataacaaggaacagccg

SEQ ID NO: 12 - DNA, 140 bp, Pseudomonas simiae
  source 1..140: mol_type = genomic DNA
caggtgactgaaccagtgaccggtggccaaccgggagcgccattggtctgcatccatgtgaactccgaagattgtcgcccagttgacagtacgccacaggctgacagggcatgatcaagcatcctacaggaggaataaca

SEQ ID NO: 13 - DNA, 140 bp, Pseudomonas simiae
  source 1..140: mol_type = genomic DNA
ccattaacggcaatggctggccgcctgcgggacattgtttgccgcaacggccacgctatccccaataaaaccgggcacctggcacccggcacgctatttgctccagctgtggcacagaacatttccacaggtgaccgatc

SEQ ID NO: 14 - DNA, 140 bp, Pseudomonas simiae
  source 1..140: mol_type = genomic DNA
ccattaatacggtcggtaaatggcccgaaaacgacctgctcatagccatctgaggcattcgtgcaatctgcccttgccgaccctttggacggcaaccagaatccaggtcaaaccccgttcccttcaccaggataaacgtc

SEQ ID NO: 15 - DNA, 140 bp, Pseudomonas simiae
  source 1..140: mol_type = genomic DNA
ccccaatagccctactccaacgcgcaaaaacaaccacacagcagcagatgacgtgtcgcaccaggtcttttttttccgccaaagaacagggatcatcgccctccccccaccatccccaaagacgccgaggaaccgagagc

SEQ ID NO: 16 - DNA, 140 bp, Pseudomonas simiae
  source 1..140: mol_type = genomic DNA
cctcgtcgaaaatcttcaaggatgttcgtattcagccgcaacgaaaacaagtcaatcatcgataagtagtgcaaacgaacaatgactacaacgaaagagcatagtagccttcctcagcgcttgaactcaggagtctgtgc

SEQ ID NO: 17 - DNA, 140 bp, Pseudomonas simiae
  source 1..140: mol_type = genomic DNA
cgcaggaaatgtttgggcatattccgcccgtagaaggttaacgcgacttttcatcgccttatccccgacatcccctctaggagcaaatcctacaggggactacaatcccctcccccgcgccattagctgatccaccccgc

SEQ ID NO: 18 - DNA, 140 bp, Pseudomonas simiae
  source 1..140: mol_type = genomic DNA
cggtggcacagggtaaggcgttcggcccaccacaatgaacttgcgacgttcaaagacagctataactcgttgtagttcttcgtttttcttataaatctacagctctgatatgctttccggccgcaggcggagatagcacc

SEQ ID NO: 19 - DNA, 140 bp, Pseudomonas simiae
  source 1..140: mol_type = genomic DNA
cgttgaggggcagtcatggagggcgctcggttcgtcaaggaaagttacactcagtagtttacttgctgtgtattttgttgcaatcttaaaactacactgtgcagtgtaattaaagatcggtctgtaggagtcacctgtaa

SEQ ID NO: 20 - DNA, 140 bp, Pseudomonas simiae
  source 1..140: mol_type = genomic DNA
cgtttgggagagtttcaattatttttgcccaggcatttcatcgccggccgcaggtttggctttatgttttgccgccaacccatgagaatgcccggcacattcaggctgaaacgctgaataacagatcccgtaggaaggtt

SEQ ID NO: 21 - DNA, 140 bp, Pseudomonas simiae
  source 1..140: mol_type = genomic DNA
ctccgcgcagcctgtcaagagtttacaaacaaccatgaacgtaagtatattgcgtagcaagctacttatctactcagtgtttgttttttacccttccacccttcttgtgcgcatcctgcgcgcctgacccgaggatcttc

SEQ ID NO: 22 - DNA, 140 bp, Pseudomonas simiae
  source 1..140: mol_type = genomic DNA
ctggattggggcattgcgcactgggatgccagccggatcccgcgcacactcacgctggccgaagcgcaattgagctgaaggcgcttgcagcttgcccggcatccgttaccatcgccgcacgtttttacacagaggccaac

SEQ ID NO: 23 - DNA, 140 bp, Pseudomonas simiae
  source 1..140: mol_type = genomic DNA
ctgtaagcaattgctgttaatcttgcgcgcccagcggcatctctgacgtagcgatcggttgaaccggacgttacagagatgctctctgggcgcgctcgtttttgagctttcgataatttcaacaagcgaggaggaccgcc

SEQ ID NO: 24 - DNA, 140 bp, Pseudomonas simiae
  source 1..140: mol_type = genomic DNA
cttagatggcctttcgctgactaatgtccggtttttctctgcttgctgtacggtcagggaggggccggtgtaggatgcgcgccgttcggcgcaggccgacacctcttttcgttccacaccctttggaacccgaaatagcc

SEQ ID NO: 25 - DNA, 140 bp, Pseudomonas simiae
  source 1..140: mol_type = genomic DNA
gatgggaggcttattctaaaaaggcaggtcgctaaacataagttacaaaacctgtttaaaacgccaatacacttttcaaagcatggtagtgaaggctataaaccgtgctttcgaaagtcacgcgcaaccggagccttatc

SEQ ID NO: 26 - DNA, 140 bp, Pseudomonas simiae
  source 1..140: mol_type = genomic DNA
gcgagccagccccaacaggaggctgcatgtttgttggtgctctgttactccaaccgtaacgatccgccgccgagcttgattgatggccccctcaatcactcctaaggtaagccccacgacagcaaacccgtggagcgacc

SEQ ID NO: 27 - DNA, 140 bp, Pseudomonas simiae
  source 1..140: mol_type = genomic DNA
gcgcaatgcttcagtgggtaggcgggataaaaaccacgtcacattctgtacaaaaagtctgccagtcgcctgatttcagcgtatgtttcaccgcaaaatcttgtagggcgattccgcccactacttaaagggagttaagc

SEQ ID NO: 28 - DNA, 140 bp, Pseudomonas simiae
  source 1..140: mol_type = genomic DNA
ggccgatcaacttcacccggcggtcggcgcacagaataagttgctggaaaatgtctggccgacgctaaaaccgctgttatgacgcttttctaccggcaggctttcggctaaggtggcgcccccccgatttggagcccccg

SEQ ID NO: 29 - DNA, 140 bp, Pseudomonas simiae
  source 1..140: mol_type = genomic DNA
ggcgatgtagtcggtgagtcgaatcaacgtcgaatcctctaacggtcagagaagttcgaaacgctagcatggacatacgacactttactatttgtctttttgtggatattttatccagcatcagacaacgaggtccgctc

SEQ ID NO: 30 - DNA, 140 bp, Pseudomonas simiae
  source 1..140: mol_type = genomic DNA
ggcgattttcgcgagcaagctcgcgcctgcagtgatcaaggcgcttttaccctttaatcactcagatcaatagtgctgttcaggttcgcccacggtcaatattatccacatcataaaccccgtcttccgtggagtgcccc

SEQ ID NO: 31 - DNA, 140 bp, Pseudomonas simiae
  source 1..140: mol_type = genomic DNA
gtcagtcagcacatgtagcagctgacccgccgccttcgcgagcaagcccgctcccacatttagatcgcgtatagcctgccacaccacaaaaccctatataatgtgcgacttttagccatcctgactcctaaggagcttta

SEQ ID NO: 32 - DNA, 140 bp, Pseudomonas simiae
  source 1..140: mol_type = genomic DNA
gtgagattgtcgtcggctgtcaggccgccttcgcaggcaagccagctcccacatttagatcgcggggccacagtgagattgtcgtcggctgtcaggccgccatccactacaatgccctccattcaaccccggggggcttc

SEQ ID NO: 33 - DNA, 140 bp, Pseudomonas simiae
  source 1..140: mol_type = genomic DNA
gtgggcatcatcaggctcggcgatctgaaattaggataaatgtacataggtgtcaataatggtcgacatgcatgtacatttttattgcggggccctgaactcatcgctaagatcggcctcaccccttaggaggacgcctg

SEQ ID NO: 34 - DNA, 140 bp, Pseudomonas simiae
  source 1..140: mol_type = genomic DNA
gtggtttctggtgctgtgcgaaacccaccatagagaacgggtaattcaccctcaaggccttgaatccattccctatcaagctatcatccaggcccagcggggcttgatagttttctatcaagacgctcaaggggcctgtc

SEQ ID NO: 35 - DNA, 140 bp, Pseudomonas simiae
  source 1..140: mol_type = genomic DNA
gttgtccactgagttatccacagcttctgtggattgtcccgagcgcttgctctagaacgggcgtgcacagttttttcagctttacctgtacgaaaaaaagagtagagtggcgcgccttccgttctgtctcacagtgcttt

SEQ ID NO: 36 - DNA, 140 bp, Pseudomonas simiae
  source 1..140: mol_type = genomic DNA
tcaggcgcagtgcgccgatgctgcgctgggcgatgcgcaacggttcagtgcaacgggcccatcggcggatggggttgatgtaggcgacggcgcatagctgttaaacttcgcgccctcatttccccagcaaggcgcgctcc

SEQ ID NO: 37 - DNA, 140 bp, Pseudomonas simiae
  source 1..140: mol_type = genomic DNA
tcctgcactcaaggattaaacgtagcagcgccatatggatttagcacgtggccttagaggaacgattgcaatcattgcaattgcaatcatatgtcagtttccgttaaggtaatgacattcgtcgccaggtgatcctctcc

SEQ ID NO: 38 - DNA, 140 bp, Pseudomonas simiae
  source 1..140: mol_type = genomic DNA
tcgcgagcaagcccgctcctacatttcgatcgcgaccgaccagtggccccttgcgcctgccgaatttacttgttagcttttggcaaaatgttaacgataacatttgctctaacaataaaaacaaaacagagaaccacgcc

SEQ ID NO: 39 - DNA, 140 bp, Pseudomonas simiae
  source 1..140: mol_type = genomic DNA
tcgcgctggactccatgcagaaaattttcctacgcatctggacaactggccatcatcgaccgtattgtctgccccgcaggccaccgcagtggccgacgtcgctcggacggttccgggcgcttacgtacttcgaggcaaca

SEQ ID NO: 40 - DNA, 140 bp, Pseudomonas simiae
  source 1..140: mol_type = genomic DNA
tcgcgtcctgatgaattgcgatggtcagggccggcagtttgccgcatgtcctctgtccaggccgcttttggcggcgttcgcgactatagcagcaatgattaagtgcttcaattccataaaaaattgttatcatcgccgcc

SEQ ID NO: 41 - DNA, 140 bp, Pseudomonas simiae
  source 1..140: mol_type = genomic DNA
tgcatgaagtggctcgccaaatgtgcggagtctaaaggtacagccgcctgcagagaaggcatggcttgacactcgcgcaatttacagaatattgttccgttacagaatattattctgttaacccattgcgaggataatcc

SEQ ID NO: 42 - DNA, 140 bp, Pseudomonas simiae
  source 1..140: mol_type = genomic DNA
tgtgatcaagccattcaagcaagtcctgccttgataacggccgcgccgagcctaatcggttcggcgtactcctaattgctcattattctcggtaacctgccgacaacattctgaatattcgtgtggaatcaggagttccg

SEQ ID NO: 43 - DNA, 140 bp, Pseudomonas simiae
  source 1..140: mol_type = genomic DNA
ttcacttggctctagattgccctttcggcgacacatttatttgaaatagtgctgtacattcatccagtactgtcctacattccatcccaggtgaccggatgtcacctgagcttcactttttaactatggatggataaaaa

SEQ ID NO: 44 - DNA, 140 bp, Pseudomonas simiae
  source 1..140: mol_type = genomic DNA
ttcctgtacgttcgtaccactgtttcagcctcgcgacggttcagcatcttgcaatgggtatggcgcctgcgtaagtatccgcaggccgactcacgaggtcgccctcaccaccaaaaatgacaacaaatcatgaggccaac

SEQ ID NO: 45 - DNA, 140 bp, Pseudomonas simiae
  source 1..140: mol_type = genomic DNA
ttcgcgagcaagcccgctcccccattgaccgcgctttgcttccatccgcgttttgttcctccatcacccagatcaatcttgctatccacccaccattcgcgcacttagactcaggccaattcctccaaaggtaagccgcc

SEQ ID NO: 46 - DNA, 140 bp, Pseudomonas simiae
  source 1..140: mol_type = genomic DNA
tttgccggaccgaactggctgtttgcgtcgatcgtgcttgcatctgcccgccccgatcagtacaaaacgccctgagccgcacggcaacgctggatccatcgaccgtcggccaaactgcctggaccctgaggaagtaagta

SEQ ID NO: 47 - DNA, 140 bp, Pseudomonas simiae
  source 1..140: mol_type = genomic DNA
aacagcctttgtcgcggattgacgctttcggcaattcccctgtcgtttttgcacccggctctgtcggcccctgggcatatgctggccccaaagcgccggcagacgattcggcgcatgaatcgccaataaggggacgcctg

SEQ ID NO: 48 - DNA, 140 bp, Pseudomonas simiae
  source 1..140: mol_type = genomic DNA
ataacggctacaaaaatctcacaaagttttcacatacccctcccgatactcccgcccgaagggccgccccatgcaagggtggcctctgttgcgcgcgcccctgcgcgtcaactgaaaaaattatcgggttggagcgctgg

SEQ ID NO: 49 - DNA, 140 bp, Pseudomonas simiae
  source 1..140: mol_type = genomic DNA
ataacggttctcattagtcgatttggttatttgcatatactcaaacatcacttttgcgcataaacgcaaactggtatcttgccccggctccgttgggagtgcgcggccgtgcgcgcagatttgccgagaacaggacctat

SEQ ID NO: 50 - DNA, 140 bp, Pseudomonas simiae
  source 1..140: mol_type = genomic DNA
cgcagacctgaatctcataaatttgttgcaagacgttaataataattactatcattcgcgacggagtcgtatccgcgcagtgaggacgcgcaacgctgttgcccttttcctcacataacggttcgccgtcaggatcgacc

SEQ ID NO: 51 - DNA, 140 bp, Pseudomonas simiae
  source 1..140: mol_type = genomic DNA
gacagttgtgcttaaggatgcgacctgaagtcgcattcgtcacttctgtaggaattgttcacttcgtgacacatctcgatactttcatgaatcttcatgaaggcgtaggctgcgcgttcatttcgccagcaccccagacc

SEQ ID NO: 52 - DNA, 140 bp, Pseudomonas simiae
  source 1..140: mol_type = genomic DNA
gcaccatcctggagcgcctgcttgtatgcacatgtctatcgccgacactccatcagtcttatcccctccgatgtttctgtttggcgcccgcgcccctataatggctgcctttttcgcccaatgattttgcggagttggtg

SEQ ID NO: 53 - DNA, 140 bp, Pseudomonas simiae
  source 1..140: mol_type = genomic DNA
taaagtcctcgccaccttgcagggtgatggggcataagccactggttatcccgtggatgttgatttttttccggcagccgtcgacagaaggtgaagctgggctataactaatctgcttttcgcaagtcatgaggtagaac

SEQ ID NO: 54 - DNA, 140 bp, Pseudomonas simiae
  source 1..140: mol_type = genomic DNA
tccttctagacagctgtggggcacagtctaacgaatcgagcagatatgtcatgtactggctcaagcgtagataaccaaatgtttcttgcaatgaaagtacatgacgtgtacttttgtttccaggccacaggagaaccgcc

SEQ ID NO: 55 - DNA, 140 bp, Pseudomonas simiae
  source 1..140: mol_type = genomic DNA
aaaaacagaacaaaaaatccaaatgaaaccgtgtcaataaaaattttgcattttgaaacactctctcctaggttctttccacttgccagcgaagcccgttggcaagccgtcctcaaacaacgtcctctaggagatacacc

SEQ ID NO: 56 - DNA, 140 bp, Pseudomonas simiae
  source 1..140: mol_type = genomic DNA
aaacttcggtgccagagggctcgagcgagacgccccgcgggctgcggccattgaccgaccggtcatcacccgggcggttttccgtcgaccacccggcattggtatttgccccacttccccgggggccatagaatgcgccg

SEQ ID NO: 57 - DNA, 140 bp, Pseudomonas simiae
  source 1..140: mol_type = genomic DNA
aaggtgacgatgaaacacaggttcgcgcacacgctgaagacctggcaaaactggtaactgaagtttgcgcctgaattcggcttgccagtggtgatgtggttgggtaacatctgcgcccactttgaccgacgaggtacagc

SEQ ID NO: 58 - DNA, 140 bp, Pseudomonas simiae
  source 1..140: mol_type = genomic DNA
acccgatgcctggactcaggcaacggggagtaaccacccgattgcgaccttttgccgtcgaaaacgacacccgccgttgacgctgccccccgatcgttattaagtgctatttatccgcattaatacgataaatcgaactt

SEQ ID NO: 59 - DNA, 140 bp, Pseudomonas simiae
  source 1..140: mol_type = genomic DNA
acgtaagtgaaggtgtcacagaacactccttgagcgagacttagatactgatcgatacaaatgttgcaactgagcgcaccgcgttgtcaacgacttatgtatcgactagtatctatatcgctagccaccggagacttgac

SEQ ID NO: 60 - DNA, 140 bp, Pseudomonas simiae
  source 1..140: mol_type = genomic DNA
agatctgagtggcacgctgataggcgccagaaaaagacatttgcaaaagtccggatattccttagaatatgcggcctttcgggcacctatgcccgctgtgcatttagatttgcagcaccgactacaggaacgatgttcac

SEQ ID NO: 61 - DNA, 140 bp, Pseudomonas simiae
  source 1..140: mol_type = genomic DNA
agttaggggtttatttcaaagatgaacttcggctatttcccatggcccctgttattcctgagggatatttaccaagcaattgtcaagtatttcctgtacgatgcctggccaagttaattttctacaggttagtggccgcc

SEQ ID NO: 62 - DNA, 140 bp, Pseudomonas simiae
  source 1..140: mol_type = genomic DNA
ataactcggtaaataactgttttaaattgtcgacgtcgttgaagacgggccgataataaacgtgctggggctgataaactccatcttaacctacgtatcgttctgatttttcagaaaaagcccttctggagcccctaccc

SEQ ID NO: 63 - DNA, 140 bp, Pseudomonas simiae
  source 1..140: mol_type = genomic DNA
caatttcgaggaaaaagtacggttaaagccttctgctgcaagactttaggcaaattgacatctggatttatatcaatatagtggtgcgggccctgcgtggggggtctgtctgatgatttgaagcataaataggaggccac

SEQ ID NO: 64 - DNA, 140 bp, Pseudomonas simiae
  source 1..140: mol_type = genomic DNA
cacggttttctgatgaaaggaggttttcagtcgaatacgcggcttgcgtgattaaaaaggtctggtagaatccgcggcctaattacgtgcggtattcgacaatagtgtcgagtggcggcacgctagcccgaggaagtgcc

SEQ ID NO: 65 - DNA, 140 bp, Pseudomonas simiae
  source 1..140: mol_type = genomic DNA
ccaatcgcgacactgtcacggcagatgtactggcgtggatcgaccaggcgctgacactgcgcaggccgactcgctgcgaataatttatcgtttagaatcaatattttaaattaccgattagccacaggatgcacctttag

SEQ ID NO: 66 - DNA, 140 bp, Pseudomonas simiae
  source 1..140: mol_type = genomic DNA
cccgcagacaatgaccgttcgtaccgccccctgaaaaattttataaatttatcttgcgcgcaaaacattagcgctatacattcacctcaccaactacttagcgcgcaaacatttagcgctaaaacaaagaggaaaacgcc

SEQ ID NO: 67 - DNA, 140 bp, Pseudomonas simiae
  source 1..140: mol_type = genomic DNA
cctggaggctgagctcccggcaattcccttgtcatacgtggggcttttcattaccatccgtcaaaatttttataagtaaagattcaacacttagtagacgcctgatttaacaggccaaaaagctgatgggagaggactga

SEQ ID NO: 68 - DNA, 140 bp, Pseudomonas simiae
  source 1..140: mol_type = genomic DNA
cgaaagaatggcgccctcaggggtgccattttttttgtgccgttttttaagcattctccgtaaccaaatacatcgcgataagctgccagccgccgccatagcaggtaggatgtacgcctattttacgggtggcatggtta

SEQ ID NO: 69 - DNA, 140 bp, Pseudomonas simiae
  source 1..140: mol_type = genomic DNA
ctcctgtggcctggaaacaaaagtacacgtcatgtactttcattgcaagaaacatttggttatctacgcttgagccagtacatgacatatctgctcgattcgttagactgtgccccacagctgtctagaaggacgtcaac

SEQ ID NO: 70 - DNA, 140 bp, Pseudomonas simiae
  source 1..140: mol_type = genomic DNA
ctgcctggtccgactgcccggtggcggtcgcagcacccggtatcaggccaagacagcgggcaagtgcgccccgctcatttgccccaatcccccatgtctgctagtgtcgcgccggtttaccgtctaccggaatagccgcc

SEQ ID NO: 71 - DNA, 140 bp, Pseudomonas simiae
  source 1..140: mol_type = genomic DNA
ctggtcgcagggttgcaccaaggtcggtacgcaagaaatgggcgacgcagtagtcgccgcgctgcggaatctgtaatctctcgggcccgcttgcagttcttgctgcaaggcggcccactttttaacaaggtgtagttgcg

SEQ ID NO: 72 - DNA, 140 bp, Pseudomonas simiae
  source 1..140: mol_type = genomic DNA
gaagaacgacggcataaggctcgcaatgggtcgaatccgaagaggccccgagcttacaccatgggggtgctcaggtctcgctgcagcgcatggaaaaatgttaattgactatgaagaacgttcgttctactgttagccac

SEQ ID NO: 73 - DNA, 140 bp, Pseudomonas simiae
  source 1..140: mol_type = genomic DNA
gcatcgcgggcagcccagatgggtttatcgcatcagcaaaatcaccgatattccccgctcaatccccagcaaagttctgataacgtgcgtctatcgattgcagccggtatgcttggcccgcaacctcatggacgcccgct

SEQ ID NO: 74 - DNA, 140 bp, Pseudomonas simiae
  source 1..140: mol_type = genomic DNA
gccaaggctgagtgtgatccgcttgaagcccggcagctagtgcgctgccgggttgattatttgttattacagcgatattatctcgcgccctatttcttggcttccggggcgtaggtagctgtcaattggagtcccactga

SEQ ID NO: 75 - DNA, 140 bp, Pseudomonas simiae
  source 1..140: mol_type = genomic DNA
gccactataaagcgccgggaaatatatttcaccccgtgacgctgtttcagaatcttcctgacctgcgaaagcatgccgttacgccgctggaacggttaggatagccgcctgtttcagtcagtctttttcgggagtagccc

SEQ ID NO: 76 - DNA, 140 bp, Pseudomonas simiae
  source 1..140: mol_type = genomic DNA
gccgtgaacaattttttaccaatatttgctagaaccccacggcaccgggataatgcgcaccactcggctattgctccctgtcccggtattcggggccaaaagccgcacatggaagcccgcaggggcgggataacttctca

SEQ ID NO: 77 - DNA, 140 bp, Pseudomonas simiae
  source 1..140: mol_type = genomic DNA
ggcggaaaatgcgtcagccgttgtcgctgaatatcgcttcacacctgatgaaaaagcacgactgatggcgcttggatagcgtttcctgcgccctggccgccgcgatttaggtaaactgcccgcccttcgcggaggccaac

SEQ ID NO: 78 - DNA, 140 bp, Pseudomonas simiae
  source 1..140: mol_type = genomic DNA
gtagccacttaaggctggttttggaacgggccacgggccacctcgaaaaataaaacgggtcgtgattaactgggcggtcccggactcgctcgggtaaactggccgccgctttttacaagatcagacgcaggggtcagatt

SEQ ID NO: 79 - DNA, 140 bp, Pseudomonas simiae
  source 1..140: mol_type = genomic DNA
gtcggctcggttgaagcgatcaccgccaaagtgctgcaagcactcagctgatcgctctgccggctacatggataacggcccgcttgcgggccgttgttgtttatactggcgcacttttttcgacttggacacctacaccg

SEQ ID NO: 80 - DNA, 140 bp, Pseudomonas simiae
  source 1..140: mol_type = genomic DNA
tggtgtcgctgtccaatcggattctcctggagcagcaggtcagtagccgctaaaccggcaaaccctgctaatgtacgccgccgctggcccggctgaccgggttgtggcggacattgacctgactgtcggcacgacttgtt

SEQ ID NO: 81 - DNA, 140 bp, Pseudomonas simiae
  source 1..140: mol_type = genomic DNA
tgtatccgtgaagctgaatctccctttatttaatagggttgtttcggcagggcgggctaaccgttatactttcgccgcctgcgaaaccgtggccgcaggcgtgaatagccaccattgagggtgaacgcgtgaacctaatt

SEQ ID NO: 82 - DNA, 140 bp, Pseudomonas simiae
  source 1..140: mol_type = genomic DNA
tttgtattggacgctgacagtggtgtagatataatgcgccacctagagagaaaggtgggtcctgccgttgctgcaagacgacaccgaacctccacccattagaaaaaaaagcctgttgagccttgagtaggagatagcct

SEQ ID NO: 83 - DNA, 140 bp, Pseudomonas simiae
  source 1..140: mol_type = genomic DNA
aaaatccatggacgacgttactccaggcaatccaagtgcctaacagaccatgtctggtcaggaatgtcgctattttatttgccgtgcaaccctggcacctttgcggcgtctaacgcccaacattacccaggagttacccg

SEQ ID NO: 84 - DNA, 140 bp, Pseudomonas simiae
  source 1..140: mol_type = genomic DNA
aagatgcggctcatgacgggtcctattgggcagtcttcaggaaggcaacaagggtatgcctcgtgccgcgaagcgtccagtcggaggaacgttgcgcccctcgcaacagtccacctcctatacggacagaggacctcccc

SEQ ID NO: 85 - DNA, 140 bp, Pseudomonas simiae
  source 1..140: mol_type = genomic DNA
aagcccaggatccggggttcaagtaagcacacaagcggcaacacttcgggaaccgaatctggcgttccgggtcgatcagtgcgccgggagcgcggtttatactcccgtcgctcgccacttcaccgattcagggttgatac

SEQ ID NO: 86 - DNA, 140 bp, Pseudomonas simiae
  source 1..140: mol_type = genomic DNA
aagcgcatgctggaatccgtcaccccctgacggccccataaagacgataaagcgaactttcctacgtcacaaacagtcacttaggatagatgcagctcccgttgaaacgattgttcccgaactattgagtggtgatcgag

SEQ ID NO: 87 - DNA, 140 bp, Pseudomonas simiae
  source 1..140: mol_type = genomic DNA
aagctggcgcagatgtatcaagctgttgcgaaaacgcaccagattccgtttttgatgtcagatgtctcgtcattcagtgaaagctgaatgccgccctcccgcgtcgctggaggcgctcttccgcggtccaggaggccgcc

SEQ ID NO: 88 - DNA, 140 bp, Pseudomonas simiae
  source 1..140: mol_type = genomic DNA
aatgcgccggggtgggtgacgtttacagtggatgattgagatgtcacgtgcgcccaggctcactcatcgcattgtcagacaatttttttgaatcttttttgacgtcaaatatctgtatttcatgagcataaggaggtctc

SEQ ID NO: 89 - DNA, 140 bp, Pseudomonas simiae
  source 1..140: mol_type = genomic DNA
aatgtctacgccagttcatttgaccgctgaggcagtggggcactcgcagagatatcccacactacctgagtcctttgccgtattggtttgcaaggtggcgcatggaagtcttgagggtgcattcactatcaggattcaac

SEQ ID NO: 90 - DNA, 140 bp, Pseudomonas simiae
  source 1..140: mol_type = genomic DNA
acagcgctgcaggtgctcaagtccgggtgattggccaagaaggcgtggtgttaagggttgaaactgtttagtcagtgagggaactcggtcgggataactacaatcataatgtacagataacccacccggagtcacccatc

SEQ ID NO: 91 - DNA, 140 bp, Pseudomonas simiae
  source 1..140: mol_type = genomic DNA
accccccgcaaaacaccctgaccacgcaactcaatatccgctgatcaatcagcttgaacgctcaataccccgggaagtcgtacccctgagacggcgattttcctgaacgccgcggatgtagaccaacccgtgaggtgttt

SEQ ID NO: 92 - DNA, 140 bp, Pseudomonas simiae
  source 1..140: mol_type = genomic DNA
actcatcctgatttcccacattctaacctccgcaggccggtggcgagaacagccgtcatggcgattgcacatcaattggaacttattcttgtttgagagcgcctaaaaagttcaggacattcccacaatggagaacgaat

SEQ ID NO: 93 - DNA, 140 bp, Pseudomonas simiae
  source 1..140: mol_type = genomic DNA
acttaatactgactattaatacaggtgactgtacttgtgcggcgcgggcaagcaccatgcttgtcatgcgccgccgttgaactacctgggctgcgccctcatctactctttctattcccttctttggtgcgtaatccgat

SEQ ID NO: 94 - DNA, 140 bp, Pseudomonas simiae
  source 1..140: mol_type = genomic DNA
agagccagataggcgagggcgccttgttccgggtatgcctgccgttgcaggcctcggcatgaattcaatcgtcacctcgacgcaacctctgcgtgaccccatggtcgtaattgcagcaactgaccggactaaggcttccc

SEQ ID NO: 95 - DNA, 140 bp, Pseudomonas simiae
  source 1..140: mol_type = genomic DNA
agcgaagcattgcgtcaggcacagatgggatgattgcgtgaagtttctttgactgttgtaggatcgataacgtcgcgaagcagtccaacactcagtaaactgccgtttttttcccgaatccttctttccgaggtttatgc

SEQ ID NO: 96 - DNA, 140 bp, Pseudomonas simiae
  source 1..140: mol_type = genomic DNA
aggcgacatcttacactgtaggtcgaatattccagattcggattttgctcaaggccgggcctaagcaaaaaaactgcaacttttgccatcgcccggcactccattgatcactgtcccattcacccgcaaggagtcacaca

SEQ ID NO: 97 - DNA, 140 bp, Pseudomonas simiae
  source 1..140: mol_type = genomic DNA
aggttaaacacgtgtaaacccaggcccttgttgcacaaattctacgaaacgttgaaccgccagaagccactacggtcaagtgaatatggcgcacgccgagccggcgcgcgcctctatttgaacgtgaccgagggcactcc

SEQ ID NO: 98 - DNA, 140 bp, Pseudomonas simiae
  source 1..140: mol_type = genomic DNA
atacggcgttaggccggcgccgccctagagggttcccactatttcgaaacgtttcacgctgaactttctcacacacgctgcgttcacttcttatgcgctgccccgtccaggggcgttgtacaacaaggagccgcccccct

SEQ ID NO: 99 - DNA, 140 bp, Pseudomonas simiae
  source 1..140: mol_type = genomic DNA
atcgctacacggcgcaattgagtcatggaaagctccctgatggttgtgggtggaatcaccaagcctagcctaggctcaatcgaacgactgccaccctggggtggtcaaccctttgaaccccagccttcggagagcgttcc

SEQ ID NO: 100 - DNA, 140 bp, Pseudomonas simiae
  source 1..140: mol_type = genomic DNA
atggttcgtactctccgacgccttgcatcctactccgcgcggcggacgctgtgcgcccaccgcccgacaaaagcgcaagattgtgcaagagcgcgcgatgggtcgcaggccacactgtggctcaatcgatggagcccgct

SEQ ID NO: 101 - DNA, 140 bp, Pseudomonas simiae
  source 1..140: mol_type = genomic DNA
caaacaggtggccaagcagtaaatgatggcgatttaatgtgatggctccctggagtgtcatcatatagacatcattctgtcgtgaaatgaaggaccgcaggctgccagcctgcggtccttttttttcgatcgagatcgcc

SEQ ID NO: 102 - DNA, 140 bp, Pseudomonas simiae
  source 1..140: mol_type = genomic DNA
caagctcaacggcgaacgccgctttgagttcgcccaactgcagaccaacagcgtggaagaagccaagcaagccctggagaaaattcacgatgccagtgatgaaatcaccgacatcaatgtgagcaaggcgctgtaagcca

SEQ ID NO: 103 - DNA, 140 bp, Pseudomonas simiae
  source 1..140: mol_type = genomic DNA
caaggtgaccgaggcggtatttgtattcgtcgccatcgacggcagcggccgcacgcgctcggttccatcgcgcgcacgttaaacctcgccgggttttgacggtcaattgccccattgcctgccatgaagagtgctttgtt

SEQ ID NO: 104 - DNA, 140 bp, Pseudomonas simiae
  source 1..140: mol_type = genomic DNA
caccttgaaagccttgaaagaacgcgctgaaaagtaagcgcagttgatcaggtgtctcctaccttgggtggaacgtttgccgagcccccaaggtccagatagaagtaagcccatacacgggttttatcgaggaggcaccg

SEQ ID NO: 105 - DNA, 140 bp, Pseudomonas simiae
  source 1..140: mol_type = genomic DNA
cagatcaccgacccggctgtagcgccccatcaccattgaacgcaccactcagtcatttggattgaacgattatttgccagcccttctctatcgcaagaccaggggccgtcggcagccccgtttatatgaggtaaaccagc

SEQ ID NO: 106 - DNA, 140 bp, Pseudomonas simiae
  source 1..140: mol_type = genomic DNA
cattgaagccatcacggctatttgccttataacttattggtctaaaactccgcggatagcactgggtcagtttctgggtacgcgccctttgggcgtattgagaatgacccctccccaacggaaaaaccggtaaggaattt

SEQ ID NO: 107 - DNA, 140 bp, Pseudomonas simiae
  source 1..140: mol_type = genomic DNA
ccaagcagtatttactgacgctggaagggacaggccaggccgaactggtgttctaaaaacgcggatcaaatgtaaaaagaaatttcaaagccaccgtgcggtccattatttcatcacccccttacaaaggaaaaacggtc

SEQ ID NO: 108 - DNA, 140 bp, Pseudomonas simiae
  source 1..140: mol_type = genomic DNA
cccagcatgcggcgttgcgcaaaaagggctgacggcggcaaaaactactactttagtactggttttcctgtctatcgtcgcattcccaagggctcgggactcacgtgtaatgcgcgcataacgataaggatcgccctgcc

SEQ ID NO: 109 - DNA, 140 bp, Pseudomonas simiae
  source 1..140: mol_type = genomic DNA
cccccatgattcaacattttaataattaggttagaaccaatcggcattttacagccctggcttcggcccctactttagaggcgaataaccgaccccctgcccggtggaggtgtccatcacgcactgaaccaggacagacc

SEQ ID NO: 110 - DNA, 140 bp, Pseudomonas simiae
  source 1..140: mol_type = genomic DNA
ccccctgtttatggctgactgtgatttttggctgaaagtttccagcgtcctacagacggcataaagatgtcgctgaaggggaacgatcaacgcccaaacccgtcagttccttacagacccttctgaaggagcccggtgtt

SEQ ID NO: 111 - DNA, 140 bp, Pseudomonas simiae
  source 1..140: mol_type = genomic DNA
cccgatggcggcatatccagctgaacttttaaccggagcccaggctcggacctattacggtcatgctcaggaagagcgcggccgcctcagaattcggatcaagcaaagagcgtccacagggcgtaaggggaagatgctcg

SEQ ID NO: 112 - DNA, 140 bp, Pseudomonas simiae
  source 1..140: mol_type = genomic DNA
cctggcggccgcaaggccagattcccgacggggtgggttaaaaaaagaaacgaattgaaattcgccagactccatatccgtggggggcagtgggtgcaggctcccgccagcagccggtattacctgacagaggaagactt

SEQ ID NO: 113 - DNA, 140 bp, Pseudomonas simiae
  source 1..140: mol_type = genomic DNA
cgacgaaataacgaatttggcgcaaggtcagggacatagcgatatctgtttttcagaagatgggtcatctgataatagatcttcccaaggggtcaagcgtcagcctacttttcctccatcacttaggcggagcgactctg

SEQ ID NO: 114 - DNA, 140 bp, Pseudomonas simiae
  source 1..140: mol_type = genomic DNA
cgcctgaaagatctctatacggcacggcccttgctgtacagcttatgagggaaacggaatttggccgatggtcaggagacgcgaactaatcccgacaccggtcgtcaatgactgcatgttcttattaggcggtacgcacc

SEQ ID NO: 115 - DNA, 140 bp, Pseudomonas simiae
  source 1..140: mol_type = genomic DNA
cgcgcctaaattattagcgcttcaaactggctgctttgttccagtcgggttcttcgtttgatcaatggtgatcaacgttactggaacgttttaacgttgcacggttcttaatccgtgtcatttgtaggaacaccaataat

SEQ ID NO: 116 - DNA, 140 bp, Pseudomonas simiae; identical to SEQ ID NO: 115
  source 1..140: mol_type = genomic DNA

SEQ ID NO: 117 - DNA, 140 bp, Pseudomonas simiae
  source 1..140: mol_type = genomic DNA
ctacagcgcgtttcgccagcacaccggccagtcgcccaaggcctacgtcaaacaaatttccctgcgtgcacgcgcgcaagacagcccctgacaccgcgctctaggatccaacctatcgaaacgaggtaggggatcccggc

SEQ ID NO: 118 - DNA, 140 bp, Pseudomonas simiae
  source 1..140: mol_type = genomic DNA
ctgacgcccggtaaataaccggatctacatgtgggagtggcttgctgtggtgagcaagcccgatcactgcaaatagttaatcgaacttctcctctgccaaccgcctcctacctgataagcccatcgcccccggcacgccc

SEQ ID NO: 119 - DNA, 140 bp, Pseudomonas simiae
  source 1..140: mol_type = genomic DNA
ctgcaactgcaccctggccagaggctgtgggcacagatcaaggccgtcgcagtcttggcgtaagcaaccttttggcacgaccgcacaccgctgcggtccatcagacataccggcctgatttttgccaaggaacctgcccc

SEQ ID NO: 120 - DNA, 140 bp, Pseudomonas simiae
  source 1..140: mol_type = genomic DNA
ctgtcgagttggtcagaaattgaggtttttccaggcaaatcggggattgggttaaacccccaggcggtttgccagtcggagttctataaactgtcttaaaaggctgtcacccagccagtcgtattcaggagtcctgtgca

SEQ ID NO: 121 - DNA, 140 bp, Pseudomonas simiae
  source 1..140: mol_type = genomic DNA
ctttaaaaacagaaaaattgcgccgcggcccgcacgacgatccagaccttatttgtagctggcccaagacttgccaacaaacgacgattaggcgagatatcattccgtttattcatgcaaaaaataaaaagaggcgtgcc

SEQ ID NO: 122 - DNA, 140 bp, Pseudomonas simiae
  source 1..140: mol_type = genomic DNA
gaactaagctgaccccgtttcgttcttctgtaggagcgagggggacccctggtccttgctcgcgaaaaaggtacatccgacaccgtgatgcgccaggaatattttcgcgagcaagctcgctcctacaaagggctgcaccc

SEQ ID NO: 123 - DNA, 140 bp, Pseudomonas simiae
  source 1..140: mol_type = genomic DNA
gaactgacgggtttgggcgttgatcgttccccttcagcgacatctttatgccgtctgtaggacgctggaaactttcagccaaaaatcacagtcagccataaacagggggccaggcccccacttcttccacaggagcaatc

SEQ ID NO: 124 - DNA, 140 bp, Pseudomonas simiae
  source 1..140: mol_type = genomic DNA
gagggcatatacccgagattcaatagtgggaccggaaatttcctactgaatcactcggtttctccgacaagatgaactttgccacgccgtctatcctcagaacccttactgacgcaaaaccaccgttcgaggagaccgct

SEQ ID NO: 125 - DNA, 140 bp, Pseudomonas simiae
  source 1..140: mol_type = genomic DNA
gagttgtgcgccgaactgggcatcaagggcgagccgtccctggaagcgctgaagatcgccgttcacgtctgaaataaaacggaacccccaccagggctggctatcctacaagtgcatgccctcactttaggagcgacacc

SEQ ID NO: 126 - DNA, 140 bp, Pseudomonas simiae
  source 1..140: mol_type = genomic DNA
gataaaaagcgggaaaaaacgctgggcattaaagcatttttcgacttggggcgtcattcgcacctgttcaacaagtcgaccgaatgcatggcgcccagcggcaatcaggcggtatgacagacaaagctttagggagtttt

SEQ ID NO: 127 - DNA, 140 bp, Pseudomonas simiae
  source 1..140: mol_type = genomic DNA
gatcaacaaacgcatcggcaacgaacggctggagaaatacgactccgcgcccaacggtcaggaacagggttgcccttgacgggtctgatcaactaaccgacagcagcggcggcctccgggtcgcgctgtgaggattggca

SEQ ID NO: 128 - DNA, 140 bp, Pseudomonas simiae
  source 1..140: mol_type = genomic DNA
gcagttggcggcgctggccaagattatccatgagaacggcggcgggcgcggcctgatatccatctgcgcggcgggcgggctgggcattaccgccatcgtcgaaaagtagctctatcaaaaacaacaacaaggagactgcc

SEQ ID NO: 129 - DNA, 140 bp, Pseudomonas simiae
  source 1..140: mol_type = genomic DNA
gcatgggggcagccagttgacttgggttaacttatcgacgccgtaaaaattcatccagcgcggttgagctgccgacactgggaacgactcgcagtagctgtcagtccaaagttacctactcattagtaaggtacagcgcg

SEQ ID NO: 130 - DNA, 140 bp, Pseudomonas simiae
  source 1..140: mol_type = genomic DNA
gccaccgtcaacgtggtgctctatgaccgcttggccaagggcaacaacacccgttcgggccccaaatactgatattttcggaacatccggcacctgcgggcagtcagctatttatcactagccctcgttggagacagatc

SEQ ID NO: 131 - DNA, 140 bp, Pseudomonas simiae
  source 1..140: mol_type = genomic DNA
gccacggctcgcaccattttcagttgggccagttccattgctcagttccagtgatttcagaagtcagaaacggttaattgtcctacgcgttgctcgggccaagactgacagcattccttctcagtattcggatgccgtcg

SEQ ID NO: 132 - DNA, 140 bp, Pseudomonas simiae
  source 1..140: mol_type = genomic DNA
gccgaagttcagctcgctcaacgtgacgtgggtggagagcagcatgaattcccatcggttcgcaattgacccaatcacattgaaccggtgacccgccacagtcctccaagctgaatagaccacggcccggtacaccttcg

SEQ ID NO: 133 - DNA, 140 bp, Pseudomonas simiae
  source 1..140: mol_type = genomic DNA
gcctgcgcttatgggctggccttgatcatctcgtgcctggtggtggctcgcttccgtcgcggcgttaagcccggcgggcaggccctgtcagcccaatccgcctgaacgacgccacccaaaccgatcaatgagataacgcg

SEQ ID NO: 134 - DNA, 140 bp, Pseudomonas simiae
  source 1..140: mol_type = genomic DNA
gcctggaagatttcgatatgacgcaagcgcatggcaagtccataaccttagtttataggtgatccatctttattcattgtcggcagcctgtcacctgcccctaggcttgaggcctctggatttaaaggtgggcggcggaa

SEQ ID NO: 135 - DNA, 140 bp, Pseudomonas simiae
  source 1..140: mol_type = genomic DNA
gcgcgcatgatagccgagttcggaaccttggacttaacgatagagtccagttattagcctgctaatgaaaacagggctaagttgatagctctgttcgtcccgtcaggtccgtagccgtgcggaccataaggagtcgtgtc

SEQ ID NO: 136 - DNA, 140 bp, Pseudomonas simiae
  source 1..140: mol_type = genomic DNA
gcggccctggatagttcaaggaaattgcccgcgccaacccggtgcaaatttgctgaaaattaatttaaacctttgccacccgacccggatcaactctcccatatcccatccttcgtacacccatcaggagacattcgatc

SEQ ID NO: 137 - DNA, 140 bp, Pseudomonas simiae
  source 1..140: mol_type = genomic DNA
gcggcgggtaaagtttctccggtcagatgcgcggacagatcgtgttacacctgatgagaaactatttagtgtcgaaagccgaacccaaggcgtagcgtgttatcgaaagcacgtactcctcgccccagagaggcttcgtc

SEQ ID NO: 138 - DNA, 140 bp, Pseudomonas simiae
  source 1..140: mol_type = genomic DNA
gcgtttttcgtgtgtgccaaatcagtgcgtgccggaaacttgcttgaagggggagaacttttgcgaaaagaccgagtctatgtttgcaagcctgatcgtttagttatgcaagcctccttaaagtacaacgaggagtgttc

SEQ ID NO: 139 - DNA, 140 bp, Pseudomonas simiae
  source 1..140: mol_type = genomic DNA
gctacgccttgggttcggctttcgacactaaatagtttctcatcaggtgtaacacgatctgtccgcgcatctgaccggagaaactttacccgccgctttcgagtcaccagaacacagcctcttcgtaaggaatcgcactt

SEQ ID NO: 140 - DNA, 140 bp, Pseudomonas simiae
  source 1..140: mol_type = genomic DNA
gctgcaaatatgcagaaagtagcgctggggcagtaccaaggtagtagccccgcgcagcgagtgcggtcttagcatggatccctaacggtcttattcacaggagtgacacgagcatgcgcgttttcaccacccggcccggc

SEQ ID NO: 141 - DNA, 140 bp, Pseudomonas simiae
  source 1..140: mol_type = genomic DNA
gctggtaatgatcgtgctggtcgtgaatatcgtgagccgtatcttcggcaagcccaagtactaagaacgggagcacctgaattttgaacgtatcaactgcgcaaaaagccgctccgtttgtcacccaggcgcctgtggtc

SEQ ID NO: 142 - DNA, 140 bp, Pseudomonas simiae
  source 1..140: mol_type = genomic DNA
ggaacgctagactcatctgatctgcgtcaactccctgtcgcttgaaactcccagaatccaccccatctataagggtatctggcaggtttcttcattcaggatgttttatagctccggcctgctcaaaaggaaggtgacta

SEQ ID NO: 143 - DNA, 140 bp, Pseudomonas simiae
  source 1..140: mol_type = genomic DNA
ggacgaccgggatcgcgaatggcaacaggagaaagaccgcgacgcaggccggacctatcgctcgcactaggtcgctttcagacatccatcgaaactaaagtgcggcgaccatggcccaaactccatagagggagaatgtc

SEQ ID NO: 144 - DNA, 140 bp, Pseudomonas simiae
  source 1..140: mol_type = genomic DNA
ggccaaggccaatggggtgacatgcccgcaccggccttgcccacgggcgcacgccgggaaatccccacctggccaaaaaagccctaggcattcgcccccgacctgacgcgggggcggatgcccgccccaacgcggggcaa

SEQ ID NO: 145 - DNA, 140 bp, Pseudomonas simiae
  source 1..140: mol_type = genomic DNA
ggcgggtgcgaggcagatcagaccaccggggtttacagcagacgattgcacgtaggtgtcggtcgatacctttgtatgacttgccagctgggtcgctcgatgctgccatccaccctcgataaacacgggacccaaagcca

SEQ ID NO: 146 - DNA, 140 bp, Pseudomonas simiae
  source 1..140: mol_type = genomic DNA
gggaaaaaccttacatgactatcgtccaaaacccgtagaacgcccactaggcaaactttccgacgaagtgcacattgaacgcccttccgcagatgtctgacgagtgtgctgggagattgattagaactgctgagaaccgt

SEQ ID NO: 147 - DNA, 140 bp, Pseudomonas simiae
  source 1..140: mol_type = genomic DNA
gggcgttgtgcgaagctttggtgcgtgaaaaattcgtgaaattccggtgttatccactggaacttctgactacacgccagcttctatagcgagatagcctgtacaaaaccatataacttcgttcggaaaggatgtttctt

SEQ ID NO: 148 - DNA, 140 bp, Pseudomonas simiae
  source 1..140: mol_type = genomic DNA
gggcttgcccgcgatgcaggcgacgcggtctgccagcccgaccgcggggatgctatcgcaggcaagccagctcccacagaaaagcactgccccattggcggtaacccaatatcgtggctgagaccacacagggagaaaac

SEQ ID NO: 149 - DNA, 140 bp, Pseudomonas simiae
  source 1..140: mol_type = genomic DNA
ggtcgttaccagggattacgtgacgtagcactcgaatatggctttgtgttcaaggcgctggggctggtttaaggaacttagcggggcggttctgtctgaacacaggactgcctactgattgatggatcaagattgctgcc

SEQ ID NO: 150 - DNA, 140 bp, Pseudomonas simiae
  source 1..140: mol_type = genomic DNA
gtagacgccttcgcaggcaagccagcgcccacatttgattgcgcccagtctgtgaagggggctcattccaatgcaacgaatggcccacaccaccctcatatgttcacgagcaatgctttacgacaacaggtaccagactt

SEQ ID NO: 151 - DNA, 140 bp, Pseudomonas simiae
  source 1..140: mol_type = genomic DNA
gtcgtaaagcaagcgtagcagtcaatgtgcgtactgcttctggcggccgtttctacctgaaagttcatatttactgcactttctgaaacggccgcagtcggtatctacacacgccactcaatcacttcaggagagtccac

SEQ ID NO: 152 - DNA, 140 bp, Pseudomonas simiae
  source 1..140: mol_type = genomic DNA
gtctaaaaataggacagatagttcccacagggaccaccggaaccatcacaagaagtatcacaccttttttgacactgccattttgcgcctcccacgaggacgcgcttcggatattaagaccttagcggaccctacaagcc

SEQ ID NO: 153 - DNA, 140 bp, Pseudomonas simiae
  source 1..140: mol_type = genomic DNA
gtgccgccagcatcaatttatgacaagactttaaaccttgtgagttgcggctagtctcaaggttaagtcgtcgcgtttatccacgcaggcgactggacactcatcaccgcctgctaaacgcgggcttcattgaggttgcc

SEQ ID NO: 154 - DNA, 140 bp, Pseudomonas simiae
  source 1..140: mol_type = genomic DNA
gtgctgccggagcagccattgccgagctacctgcacaaaatcagctgaattcatgcaatttgttgctaacttcacccgaccattgaaaccaccgctaccccgccccatctaacttgcatacttccttatgcaggtgcccc

SEQ ID NO: 155 - DNA, 140 bp, Pseudomonas simiae
  source 1..140: mol_type = genomic DNA
taagcaattgggtacagaccttacacaacctttacctagcggctgtgctgtttttcagaaatacgttgtcttaaaagtatccccgcgacattacttgagtctattgattgcagaccacaagcgctggggctaaccgagga

SEQ ID NO: 156 - DNA, 140 bp, Pseudomonas simiae
  source 1..140: mol_type = genomic DNA
taatgttgatccgcgaaatcgagggtccggacaaaagttccggttaggcagcgatttatatatctatggaataaaaggaactaatgccgaaccgccatgctcgaaaggccagtagatatcttcaataaggagaaacaccc

SEQ ID NO: 157 - DNA, 140 bp, Pseudomonas simiae
  source 1..140: mol_type = genomic DNA
taccagagtgctggctgagccgtttcagcccatgatcaagtgcaaaagattcaaccttgcgacattctcttaacagatcgacaagccacgcaccgttccgtcgcacttaatcggagcgcacgcacattaatggagcagac

SEQ ID NO: 158 - DNA, 140 bp, Pseudomonas simiae
  source 1..140: mol_type = genomic DNA
taccgcgcgatgtcgttgaggtggcgcagtatcctgcaacgacattgttatgaccacccatcgcctcgaacacatccaatcgaacctatccagacgagttctactccacctgtatgaaccctgatggtggagtaagaatc

SEQ ID NO: 159 - DNA, 140 bp, Pseudomonas simiae
  source 1..140: mol_type = genomic DNA
tcaaaggtcataaaaaaggaacggcccaaaaccccatcagtcactacttaagcaactcgatttcccaggtgccgtgcctggggttttgctcaaacgagccggggaccgctctggccactttgaataaggagttttgtctg

SEQ ID NO: 160 - DNA, 140 bp, Pseudomonas simiae
  source 1..140: mol_type = genomic DNA
tcaagccaccaagcgtccatcgaagctgtcctttgtcatggttggtaaaggatggacagcggtgaccgccagtcagttgcctggaacccccacacattgctgccccctaatcctcatagcccatgaataaggagaacggc

SEQ ID NO: 161 - DNA, 140 bp, Pseudomonas simiae
  source 1..140: mol_type = genomic DNA
tcacccgcaacggcacccggtggtggtgacgaggccacaggcgcagcgttcaccattcgtctaatgcccggcactctgaagaatgtactgtctctgaataataagcacggataccccgagcgtaagaaaggagagcccgt

SEQ ID NO: 162 - DNA, 140 bp, Pseudomonas simiae
  source 1..140: mol_type = genomic DNA
tcaggttgagtcgtgtagtggttggaaaacctggcgccgtacacgttcaaaacggtttcgcttgaacattgattgatgtgaaaggtctttttttggaacgggattgagagcgcaatcgctcgtgcgcaggagcaattatc

SEQ ID NO: 163 - DNA, 140 bp, Pseudomonas simiae
  source 1..140: mol_type = genomic DNA
tcataaggctgttcctggcaagtcttgaagtcatttcatggactctggtatgccgattgggttcctcgaccgacctggaacaatgtgccggatgcccagtcaaacgcctgctttcacccctatacccagaggttgtttcg

SEQ ID NO: 164 - DNA, 140 bp, Pseudomonas simiae
  source 1..140: mol_type = genomic DNA
tcatcgaaacaacctctgggtataggggtgaaagcaggcgtttgactgggcatccggcacattgttccaggtcggtcgaggaacccaatcggcataccagagtccatgaaatgacttcaagacttgccaggaacagcctt

SEQ ID NO: 165 - DNA, 140 bp, Pseudomonas simiae
  source 1..140: mol_type = genomic DNA
tccaggctgacgggcataacttgcctctgattgttgtttttagcggattttcatcccgtggtgcgggaattagcggcaaatatcgcaaagttcatcttcaattatgagcatagcattgtaggaaataaggagcgcccgac

SEQ ID NO: 166 - DNA, 140 bp, Pseudomonas simiae
  source 1..140: mol_type = genomic DNA
tcgcctggccaaccttaatacgccggagctgttacagacgccatcgggcgtgtcagaatgaacctcgcacaaggaactttgtcggcgctgcacgcgtcacaaggtcagctattaaaaagtattctctcggagacagactc

SEQ ID NO: 167 - DNA, 140 bp, Pseudomonas simiae
  source 1..140: mol_type = genomic DNA
tgaaaagggtttttgccgagaattgtcgccaaatataggcagttatatagtgaaatgcactggcaattgttttttacactgcgctcatcattgctcgaataacacacgtcagcggccatgcccgcgatgaggatataaaa

SEQ ID NO: 168 - DNA, 140 bp, Pseudomonas simiae
  source 1..140: mol_type = genomic DNA
tgaaagcgccgcgtattcggcgggtatgccacttttttaagcgtcgtagcagtgcttaactgaaaaaaccggcctcaatcgacgggcaccgtgcaggagcgagcttcgatcgaaatgggcctttttccaggaggcccgtc

SEQ ID NO: 169 - DNA, 140 bp, Pseudomonas simiae
  source 1..140: mol_type = genomic DNA
tgaacgaccgcaattgtttacctttgatagcttgcagtgtcgacctgtgcggccgcagcctctgttattcaaggcgcccggcacattccgctgaaacatttgctggtctgtacaatcacacttaaaaaggagccttaaca

SEQ ID NO: 170 - DNA, 140 bp, Pseudomonas simiae
  source 1..140: mol_type = genomic DNA
tgatcctgcaaaaaaaacagaacccctgcgtgccggcgtagtcatctgcataggcctcgacaaacgaggctcatcagtgatgtcgctggcagcccctgaacgccagcagcgtcgagcccttttgcgcaaggagataccct

SEQ ID NO: 171 - DNA, 140 bp, Pseudomonas simiae
  source 1..140: mol_type = genomic DNA
tggatgcaggaggaagtcaggctgtccggtcacgccttgtaaaatccgataaaactttttgtgttgcgcgggactcacaacttggagcgatcacgccggcagagcgtggcgcaaaaccggattagtaccaggagattgag

SEQ ID NO: 172 - DNA, 140 bp, Pseudomonas simiae
  source 1..140: mol_type = genomic DNA
tggcagggatattcagtggaggtcgccgatacccggaaagttcacgggaatgacgctcgtctcctgggctgaacggccagtgatgcggcgggtcacacctttaacgggctaccttccatccgcccgcaggagaaatcagg

SEQ ID NO: 173 - DNA, 140 bp, Pseudomonas simiae
  source 1..140: mol_type = genomic DNA
tggcgactgaacctgtcgtgcaggttgcatggtcggtgcggttgcacgttgcattccacaaccccgggacattcgcgcaaattttaatgaaccctcgcgctaaacacttgacctaacgtgtacgacctaggaggtgaagc

SEQ ID NO: 174 - DNA, 140 bp, Pseudomonas simiae
  source 1..140: mol_type = genomic DNA
tggcgagcctgcggcttgccgtccccgctcaaatgcaccaggtcatctagtgcattcgtaccccgtattgcggattttctttgcactatcctgaggtgcggatgtcgtactttccatgcgcccaaaaccccggagatctc

SEQ ID NO: 175 - DNA, 140 bp, Pseudomonas simiae
  source 1..140: mol_type = genomic DNA
tttggcaacaagcccgctggatttgacgatgaccgttcgtcgcactggcgagttttttctaaacctttgccgccctgaaaattcgaattgagggcggggtgtgttcccccgcccttatttttagcctggaggaacccatc

SEQ ID NO: 176 - DNA, 140 bp, Pseudomonas simiae
  source 1..140: mol_type = genomic DNA
tcaaatggatagctgcatttgcaatgaaaagttgcgtattacccagagtagccagtttgtccagcgcgctaaagcagcctgctgctttattgaacaaacaatcaaacagcgaagtctggagtgcaatgagggatgttttt

SEQ ID NO: 177 - DNA, 140 bp, Pseudomonas simiae
  source 1..140: mol_type = genomic DNA
tgacgccaaggaaatgtcaaatctgaggcatgatgcggcctctttgcaattcaggttgaactttggtcgtgagccttgtcttaacactcatcttgcggccaattccaaaaaccgctcccctgaactaaccggttaaatat

SEQ ID NO: 178 - DNA, 140 bp, Pseudomonas simiae
  source 1..140: mol_type = genomic DNA
ttagaagctagaccattgcgaggggtcttgtcatgtccggggtcaatacagccccgttcgtcgcagcgcgggcgtttggatcaaacctttgccgccccctgccctccactctagatagccaaccgagggagaccggtttc

SEQ ID NO: 179 - DNA, 140 bp, Pseudomonas simiae
  source 1..140: mol_type = genomic DNA
tgaacaggcctgtcgcccaaaccgaacgctgcctggttgttggggtcctaaccctaggtgtcgaggtttttaccgccccgccctccccgcgcgcattgagggtctagcgatgtgcgtacccccgagaaggagacgcttac

SEQ ID NO: 180 - DNA, 140 bp, Pseudomonas simiae
  source 1..140: mol_type = genomic DNA
tttgaacgtaatcgttcctacgcggcgtgcgggaacgatcaagcgccctcatcccatcgcatccccctacccagtacccaagtagcataggtcccaacccccgctcttcgtaaactcatgcctcagagggggaggcaacg

SEQ ID NO: 181 - DNA, 140 bp, Pseudomonas simiae
  source 1..140: mol_type = genomic DNA
caccgaagtagttaatgtcagattaatgtggtttcgtttgtattgaccgtattggtggtttagggaacttgtcctacggttcccggtcaattttcgcagtgcttcactgcacgagttatgtgagtcggaggttttttcgt

SEQ ID NO: 182 - DNA, 140 bp, Pseudomonas simiae
  source 1..140: mol_type = genomic DNA
cctgcgatggctcggcggaacgagccatcgctgacgtcgatagtcaccatcagcccaatgaagttctcttaaaaaatgcaacgcgtaacatcaaccccataccagcaacaacccaactaaaacactcggagcacaccctc

SEQ ID NO: 183 - DNA, 140 bp, Pseudomonas simiae
  source 1..140: mol_type = genomic DNA
ctatgggtcagcagtgacatggcgcaatccttcggcacgggataaaggcgacactatgccgcgccaacgagcagattgtcattttgaatgaaccttttagaacgctgccctatccatctataacaacacagggaggcaac

SEQ ID NO: 184 - DNA, 140 bp, Pseudomonas simiae
  source 1..140: mol_type = genomic DNA
ctgtcatgacgcgactccaaacggttcggcgactatgctgttttttaaatcaatcaaccttaaaaaacaacttaacaaatcaatcagccggagcaacactcggttcattggctggcccacaaacaataaaagaggtgcat

SEQ ID NO: 185 - DNA, 140 bp, Pseudomonas simiae
  source 1..140: mol_type = genomic DNA
gagttgcagcgcattttcgcggttaccggcacacaaaatcttacaggtgacacaagaccgttcgtcgccatcaaggcacgaacacgaaacgagggctgaccaatagtaggtaaggacattaatcatatggagacaccctc

SEQ ID NO: 186 - DNA, 140 bp, Pseudomonas simiae
  source 1..140: mol_type = genomic DNA
gccagcgcggcgtcaccctcccacgcgcttgatcgaaagctcctacgcgcaaagggaattagtctgcataaagtgactctatctaggtgtactgcctcggcacgtgtaaaacgcgccgagccacgcttgaggagtacctt

SEQ ID NO: 187 - DNA, 140 bp, Pseudomonas simiae
  source 1..140: mol_type = genomic DNA
gcggccaccgccgcaaaagaatccgccaaggccctgaaggccgccgtggattccggggcactcaattcctgataatccgatagaactctgctgcaccacaaccctcccatgcataacgaccaccctgcatggagaacgaa

SEQ ID NO: 188 - DNA, 140 bp, Pseudomonas simiae
  source 1..140: mol_type = genomic DNA
gcgggcggatggaaggtagcccgttaaaggtgtgacccgccgcatcactggccgttcagcccaggagacgagcgtcattcccgtgaactttccgggtatcggcgacctccactgaatatccctgccaaggaggtccaccc

SEQ ID NO: 189 - DNA, 140 bp, Pseudomonas simiae
  source 1..140: mol_type = genomic DNA
gggcgcgggtccagctggcgagggaagtgggttcggtcatggcgattggcgcttgcggtcaacaggttggcgtgtggagctgatgccctggcggaacgcttgagccaggatgcagctatcaatcaccagaggatggaagc

SEQ ID NO: 190 - DNA, 140 bp, Pseudomonas simiae
  source 1..140: mol_type = genomic DNA
gggtgccgaagacctgaagaagtaacgaacctgcgcttcgcgcatcgcgggcaagcccgctcccacattggatcgcggtcccctgtgggcgccgggcttgcccgcgatgaggccctcacagccgactaagatccataacc

SEQ ID NO: 191 - DNA, 140 bp, Pseudomonas simiae
  source 1..140: mol_type = genomic DNA
gggtggcaaaggtttaaattaattttcagcaaatttgcaccgggttggcgcgggcaatttccttgaactatccagggccgcaggcttctgctgcataggcgctcgctatttttacccgcgccattgattggagaaccacc

SEQ ID NO: 192 - DNA, 140 bp, Pseudomonas simiae
  source 1..140: mol_type = genomic DNA
gggttggcggcaaaacataaagccaaacctgcggccggcgatgaaatgcctgggcaaaaataattgaaactctcccaaacggccttgggtcaatctttaggtaatgaccaaaacccttgattgttctcaggagaaatacc

SEQ ID NO: 193 - DNA, 140 bp, Pseudomonas simiae
  source 1..140: mol_type = genomic DNA
ggtcgagtgatcggaacgctttcactgaagcgctttacagcaaagtcggaagattggcacgggagtagactcctgctgtcacacctgatgcacgctaaaaaaatactagataggaccgaggccaacccgtgaacccagac

SEQ ID NO: 194 - DNA, 140 bp, Pseudomonas simiae
  source 1..140: mol_type = genomic DNA
gttcacccttgaagaagccccgaaacgtttcagcgttttcggggtttttttgttttcgttcatcgggaaccagacacctcttttcccttctaagcttctggcagatacccacgctgcttcgtccctccaggagtttttcc

SEQ ID NO: 195 - DNA, 140 bp, Pseudomonas simiae
  source 1..140: mol_type = genomic DNA
tcagggaaagcctcaggaatgatgcgcagttttgtgactgacaacccttcgtccaggtcacgccgcacgcaaattagcttgaatctttgggacgcgcagcctgtccacacactataagccccacagtatgaggattggcc

SEQ ID NO: 196 - DNA, 140 bp, Pseudomonas simiae
  source 1..140: mol_type = genomic DNA
tgcccgaccgcccagattcagcgcaggcctcagggcctgcgcaccaaggcaaagcaaaagctgtaccacacaattgcactgaaccttcaccgaagccggtgactcgaaaacgtacagatcagccaatcacggagataccc

SEQ ID NO: 197 - DNA, 55 bp, Pseudomonas simiae
  source 1..55: mol_type = genomic DNA
ctctttccctacacgacgctcttccgatctttactggatctatcaacaggagtcc

SEQ ID NO: 198 - DNA, 60 bp, Pseudomonas simiae
  source 1..60: mol_type = genomic DNA
ctctttccctacacgacgctcttccgatctctgtgccaagcgaactgagtgtatttgagc

SEQ ID NO: 199 - DNA, 49 bp, Pseudomonas simiae
  source 1..49: mol_type = genomic DNA
gagttcagacgtgtgctcttccgatctggtacctttctcctctttaatg

SEQ ID NO: 200 - DNA, 54 bp, Pseudomonas simiae
  source 1..54: mol_type = genomic DNA
gagttcagacgtgtgctcttccgatcggcagtttaccagtagtacagatgaact

SEQ ID NO: 201 - DNA, 28 bp, Pseudomonas simiae
  source 1..28: mol_type = genomic DNA
ggcagtttaccagtagtacagatgaact

SEQ ID NO: 202 - DNA, 140 bp, Pseudomonas simiae
  source 1..140: mol_type = genomic DNA
aaaaatccgcgaactgttcagaccagtccgaccatcgcaacgcaataaatccctggcttgcaggctttattgagagtgatggcatgggctgtgcactgagcaaatccagaaaattccttcgcccttctcggagagttgcc

SEQ ID NO: 203 - DNA, 140 bp, Pseudomonas simiae
  source 1..140: mol_type = genomic DNA
aaaggtaaatgaccggtagatatctaattaaaggaaaagctggttggaaattgacctagaggcttgctttctctagaatcgccggtctcttaaaacgggggccattccggcccgttgtggacgaaccaggtaacaacgcc

SEQ ID NO: 204 - DNA, 140 bp, Pseudomonas simiae
  source 1..140: mol_type = genomic DNA
aggtgatcgacgtggacgatttcagcaaggaagagctgacccaggctgagggtaaagtccgctgatcccagtggtcatataaatacgggcgcgagagcgatatactctcgcgcccgtttttgctttagaggacttatctc

SEQ ID NO: 205 - DNA, 140 bp, Pseudomonas simiae; identical to SEQ ID NO: 102
  source 1..140: mol_type = genomic DNA

SEQ ID NO: 206 - DNA, 140 bp, Pseudomonas simiae
  source 1..140: mol_type = genomic DNA
ccgccttcgcgagcaagcccgctcccacatttgttttgtgatgaccacacaaattgctgtacatccatacagataaaggttgatcaaagccctccgactgagcatcctgcaaggcatcaacccgatctgatgtgatgacc

SEQ ID NO: 207 - DNA, 140 bp, Pseudomonas simiae
  source 1..140: mol_type = genomic DNA
tcgcagcctcgcgaacgctcgacagctcccacccgacagcaactgtacggataaatccgcacgcagctgtagacccttgatcggtgagggatgtggctagatgggcttcccttgaagccctctagattcggttcttctcc

SEQ ID NO: 208 - DNA, 140 bp, Pseudomonas simiae
  source 1..140: mol_type = genomic DNA
tgccggtaccaccctggccaacattgccgcctgggccgatgggcgggcgcagaaccttgtcgaaggatgatcagcggtcacataccgggctcatgatgagccagcaccgtgataggatgccgcgcctatttggaggatcc

SEQ ID NO: 209 - DNA, 140 bp, Pseudomonas simiae
  source 1..140: mol_type = genomic DNA
tgcgatacattgctaattgcactaaatttacattctttacactttttccttacggtttttacggactcgttcgtcccggcgttttacagggcggataagaatcaataccattggcgcctttccgaattcaggtattgccc

SEQ ID NO: 210 - DNA, 140 bp, Pseudomonas simiae
  source 1..140: mol_type = genomic DNA
tgcgccgtccagtgatccctaccaacggattatcgcctgaacggtaacaaagattcacaggaatgccagcttatcgtcctccctatcgccccctagaattgcgccacctacccccgcatcacctcaggaagtcgcttgcc

SEQ ID NO: 211 - DNA, 140 bp, Pseudomonas simiae
  source 1..140: mol_type = genomic DNA
aacatcgtttgaatcgctaaacactctaaagagctgtcaacactcacaggttactgaaccgacgcaccctgctagcctgagccagtcgaactcaatcctcgtctggctcacgcctttaccatgtgcgaaaggaatcgtcc

SEQ ID NO: 212 - DNA, 140 bp, Pseudomonas simiae
  source 1..140: mol_type = genomic DNA
aatgaccgctgtccctgctggaaatcgatacctgccacaccgcagatccaatgtgggagcaagcccactcccacagttggccggcggtgtggccggctatgagttcgagaggaaatggccgcacaaatggagaggactgt

SEQ ID NO: 213 - DNA, 140 bp, Pseudomonas simiae
  source 1..140: mol_type = genomic DNA
acaataagcactttgtattgtgtacaaaaaaccctaaagatttaccggctacgggccgaaacagcagtgatagcaaatcgccgcaaacctgatacaggcgcgcgacgatttggccgcccttcacgctgatggtcgtctcg

SEQ ID NO: 214 - DNA, 140 bp, Pseudomonas simiae
  source 1..140: mol_type = genomic DNA
acatttgcccctcagcctgaagaccgggtcccacttttctgctcataagctaattcgaaaacgttatttattttcggattcttagatcatttagtctcctcgcatgccgaccctcggccgcctgattgaggagcttcccc

SEQ ID NO: 215 - DNA, 140 bp, Pseudomonas simiae; identical to SEQ ID NO: 91
  source 1..140: mol_type = genomic DNA

SEQ ID NO: 216 - DNA, 140 bp, Pseudomonas simiae; identical to SEQ ID NO: 8
  source 1..140: mol_type = genomic DNA

SEQ ID NO: 217 - DNA, 140 bp, Pseudomonas simiae; identical to SEQ ID NO: 61
  source 1..140: mol_type = genomic DNA

SEQ ID NO: 218 - DNA, 140 bp, Pseudomonas simiae
  source 1..140: mol_type = genomic DNA
atgccgggatcgtcgtgaagggctgcgacgcgatggaataaagcgatgccagtttgatgtccgctgggcgggatcgaactacttatagtttggtacgacggtaccaatatcttctgcctgacagatttagaggaacgacc

SEQ ID NO: 219 - DNA, 140 bp, Pseudomonas simiae; identical to SEQ ID NO: 10
  source 1..140: mol_type = genomic DNA

SEQ ID NO: 220 - DNA, 140 bp, Pseudomonas simiae
  source 1..140: mol_type = genomic DNA
ccagcaagctggctcgtactctggccgcagttcgcgagcaaaaagaagctgccgcagcctaaggctgagcgacttctctcgcgtatttttgtttatttcgatggccgagtaggccgtcccccaattcaggaaatacagca

SEQ ID NO: 221 - DNA, 140 bp, Pseudomonas simiae
  source 1..140: mol_type = genomic DNA
ccatcgccttacggcgagaatgagtttttccgtaaggccgtgcgtcgtcacgagctgctttgggtgctgaagcaggactgaaattgacttttttcgtcgggctgcgtgcgcgtagcagcccgattgcctcatggagtgat

SEQ ID NO: 222 - DNA, 140 bp, Pseudomonas simiae
  source 1..140: mol_type = genomic DNA
ctgtgaatctgtaaagttcaagctttctgcaaaaaaacgcaaatagagctgaattatttcatgtccgcttgtatctgaactgacagagcggtgccatcgcaacgatggcacctcccctgttctgctttagagtgctccgc

SEQ ID NO: 223 - DNA, 140 bp, Pseudomonas simiae
  source 1..140: mol_type = genomic DNA
gatccactgcggcaactggcgatgatccgtgccacggccgagctgtgcgttcgacctacttgatttgaggaaccgtcctgcatcttcacctgggccgacagccatagcctgagcgttcactgaccgaacaggatggaacc

SEQ ID NO: 224 - DNA, 140 bp, Pseudomonas simiae
  source 1..140: mol_type = genomic DNA
ggcaaggctatccttaatgccttgccgccgttcaggcgcgaaatttcttaagccacagtgggcggattcgcccacttcgtggtctatctgactgtcattcatcaggccattcatcggccgcctgggagaaccttgctcgt

SEQ ID NO: 225 - DNA, 140 bp, Pseudomonas simiae
  source 1..140: mol_type = genomic DNA
ggcatccgccctgtttggttccaccacgggcctttccacaacggcgactgcatgcgcacgaaaaaaccactcaactgtattttgacgctcgcttgacccactgttagtgtccggctccatccctacacggagtgctttcg

SEQ ID NO: 226 - DNA, 140 bp, Pseudomonas simiae; identical to SEQ ID NO: 191
  source 1..140: mol_type = genomic DNA

SEQ ID NO: 227 - DNA, 140 bp, Pseudomonas simiae
  source 1..140: mol_type = genomic DNA
gtgccgcagatctggatcggcgccaagcatatcggtggttgcgatgacctgttcgccctggagcgcgcgggtaaactcgatgcgctgctgctcgtctgatgcctaaaccctaaaagaccccaagatcacaaggatctgcg

SEQ ID NO: 228 - DNA, 140 bp, Pseudomonas simiae
  source 1..140: mol_type = genomic DNA
tgacctggtccaaacctgcttacactgatctgcgcatcggtttcgaagtcaccatgtacttcgccagccgttaatccgctgggtaatacaacgcctcggttcgcccggggcgtttttgttttgagcttgatggagctgcc